(19) United States
(12) Patent Application Publication
(10) Pub. No.: US 2023/0028696 A1
(43) Pub. Date: Jan. 26, 2023

(54) Predictive Markers of Psychosis

(71) Applicants: THE ROYAL COLLEGE OF SURGEONS IN IRELAND, Dublin (IE); UNIVERSITY COLLEGE DUBLIN, Dublin (IE)

(72) Inventors: David COTTER, Dublin (IE); David MONGAN, Dublin (IE); Mary CANNON, Dublin (IE); Gerard CAGNEY, Dublin (IE)

(73) Assignees: THE ROYAL COLLEGE OF SURGEONS IN IRELAND, Dublin (IE); UNIVERSITY COLLEGE DUBLIN, Dublin (IE)

(21) Appl. No.: 17/788,445

(22) PCT Filed: Dec. 23, 2020
(86) PCT No.: PCT/EP2020/087763

§ 371 Date: Jun. 23, 2022

(30) Foreign Application Priority Data
Dec. 23, 2019 (GB) 1919155.0
Jun. 11, 2020 (GB) 2008918.1

(51) Int. Cl. G01N 33/68

(52) U.S. Cl. CPC G01N 33/6896; G01N 2800/30; G01N 2800/50

(57) ABSTRACT

The invention relates to a method of determining the likelihood of an individual transitioning to a first episode of psychosis (FEP), the method comprising determining the level of selected markers in a bodily fluid sample from the individual, wherein the increase or decrease in the markers is predictive of the individual transitioning to a first episode of psychosis (FEP). The invention also relates to a method of predicting the functional outcome for an individual following a first episode of psychosis (FEP), the method comprising determining the level of selected markers in a bodily fluid sample from the individual, wherein the increase or decrease in the markers is predictive of an increased risk of functional disability outcome for the individual.

The present invention relates to methods of determining the likelihood of an individual, such as an ultra-high risk (UHR) individual for psychosis transitioning to a first episode of psychosis (FEP); and predicting the functional outcome of a FEP; and related treatments, compositions and kits.

Early identification and treatment of patients with psychotic disorders significantly improves their clinical outcome.¹ Over the last decade, there has been a focus on the ‘ultra high risk’ (UHR) state for psychosis² with the aim of identifying vulnerable individuals and offering preventative interventions.^{3, 4} 16-35% of UHR subjects go on to transition to first episode of psychosis (FEP);^{5, 6} 50-65% develop non-psychotic mental disorders such as depression and anxiety.^{2, 7} The clinical value of identifying those who will transition to psychotic disorder is significant and the focus of many studies.^{6, 8} Although a minority of UHR individuals transition to FEP, the accurate identification of those at greatest risk of transition would facilitate targeting and testing of preventative interventions. Clinical data have shown some value in short vector machine (SVM) models for prediction of transition and functional outcome in UHR, although the overall accuracy of such models was limited (64.6% and 62.5% respectively).⁴⁵ Improved accuracy has been achieved by incorporating neuroimaging^{46, 47} and neurocognitive⁴⁸ data. However, blood-based tests have the advantage of greater accessibility. A previous investigation of the North American Psychosis Longitudinal Study found a panel of 15 proteins that may distinguish between UHR individuals who did and did not transition, with AUC (area under the receiver-operating characteristic curve) 0.88.⁴⁹ However, a test to provide sufficient confidence in predicting UHR individuals transitioning to FEP is still desirable in the clinic.

An aim of the present invention is to provide an improved, more efficient, method of predicting transition to first episode psychosis and/or functional outcome in individuals, including ultra high-risk (UHR) individuals.

According to a first aspect of the present invention, there is provided a method of determining the likelihood of an individual transitioning to a first episode of psychosis (FEP), the method comprising:
determining the level of markers in a bodily fluid sample from the individual, wherein the markers are selected from one or more proteins of Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III, and N-acetylmuramoyl-L-alanine amidase,
wherein an increase in the level of one or more markers selected from Complement component 8 alpha chain, Complement component 6, Retinol-binding protein 4, Beta-crystallin B2, Vitamin D binding protein, Inter-alpha-trypsin inhibitor heavy chain H1, Fibulin-1, Clusterin, L-lactate dehydrogenase B chain, Complement C1q subcomponent subunit C, and Alpha-crystallin A chain, coagulation factor XII, Carboxypeptidase N subunit 2, Alpha 1 anti-chymotrypsin, Plasminogen, Monocyte differentiation antigen CD14, Attractin, Zinc alpha-2-glycoprotein, Extracellular matrix protein 1, Complement C1s subcomponent, Ceruloplasmin, Antithrombin III and Complement Factor I; and/or a decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, A disintegrin and metalloproteinase with thrombospondin motifs 13, Immunoglobulin lambda constant 3, and N-acetylmuramoyl-L-alanine amidase; is predictive of the individual transitioning to a first episode of psychosis (FEP).

According to a second aspect of the present invention, there is provided a method of predicting the functional outcome for an individual following a first episode of psychosis (FEP), the method comprising:
determining the level of markers in a bodily fluid sample from the individual, wherein the markers are selected from one or more proteins of
Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C,
wherein an increase in the level of one or more markers selected from Fetuin-B, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C; and/or a decrease in the level of one or more markers selected from Alpha-2-macroglobulin, Phospholipid transfer protein, Immunoglobulin heavy constant mu, CD5 antigen-like, and Pyruvate kinase, is predictive of an increased risk of functional disability outcome for the individual.

According to an alternative embodiment of the second aspect of the present invention, there is provided a method of predicting the functional outcome for an individual following a first episode of psychosis (FEP), the method comprising:
determining the level of markers in a bodily fluid sample from the individual, wherein the markers are selected from one or more proteins of
Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor,
wherein an increase in the level of one or more markers selected from Fetuin-B, Inter-alpha-trypsin inhibitor heavy chain H1, Insulin-like growth factor-binding protein 3, Clusterin, Galectin-3-binding protein, Pigment epithelium-derived factor, Kininogen-1, Apolipoprotein C-III, and von Willebrand factor; and/or a decrease in the level of one or more markers selected from Alpha-2-macroglobulin, Phospholipid transfer protein, Immunoglobulin heavy constant mu, CD5 antigen-like, Pyruvate kinase, and Triosephosphate isomerase, is predictive of an increased risk of functional disability outcome for the individual.

The invention has advantageously made use of machine learning algorithms incorporating clinical and proteomic data to accurately predict transition outcome to psychosis for individuals, including UHR individuals (AUC 0.95). The identified proteomic features were of greater predictive value than clinical features alone. Separate models are also provided by the invention for prediction of future functional status (AUC 0.72). Such results have clinical and pathogenetic implications in relation to management of individuals and their treatment for, or prevention of, psychosis developing, particularly from the UHR state.

### The Individual

In an embodiment according to the first or second aspect, the individual may be an ultra-high risk (UHR) individual for psychosis. The individual may or may not have been previously identified as an ultra-high risk (UHR) individual for psychosis. In another embodiment, the individual may not have been previously identified as at risk of psychosis. Where the individual is an UHR individual for psychosis, the individual may have been previously diagnosed with being an UHR individual for psychosis. In another embodiment, the individual may have been suspected of being an UHR individual for psychosis. In another embodiment, the individual may have clinical symptoms of an UHR individual for psychosis. In another embodiment, the individual may have been expected to develop a FEP under established clinical assessment criteria (e.g. prior to testing under the method according to the invention).

In one embodiment, the prediction for transitioning to a first episode of psychosis (FEP) and/or the prediction of the functional outcome may be for a period within 1-6 years (i.e. from testing). In another embodiment, the prediction for transitioning to a first episode of psychosis (FEP) and/or the prediction of the functional outcome may be for a period within 2 years (i.e. from testing). In another embodiment, the prediction for transitioning to a first episode of psychosis (FEP) and/or the prediction of the functional outcome may be for a period within 3 years (i.e. from testing).

The methods herein may further comprise the assessment of clinical features. In particular, the methods herein may be used alongside established assessment of clinical features presented by the individual. The prediction for transitioning to a first episode of psychosis (FEP) and/or developing a functional disability may have a greater predictive value than clinical features alone.

In one embodiment according to the second aspect, the method may comprise the further assessment of one or more, or all, of the clinical features selected from BPRS: suspiciousness, SANS: impersistence at work or school, SANS: increased latency of response, SANS: blocking, SANS: grooming and hygiene, BPRS: excitement, SANS: sexual activity, and MADRS: suicidal thoughts. The clinical features of one or more of BPRS: suspiciousness, SANS: impersistence at work or school, SANS: increased latency of response, SANS: blocking, SANS: grooming and hygiene, and BPRS: excitement, may be an increase in such features, such as at least a 0.09 or 1 fold increase in the clinical assessment score. The clinical features of SANS: sexual activity and/or MADRS: suicidal thoughts, may be a decrease in such features, such as at least a 0.09 fold decrease in the clinical assessment score.

In one embodiment, the individual is a human. In another embodiment, the individual is a human between the ages of 18 and 35 years. In another embodiment, the individual is a human between the ages of 12 and 35 years.

### The Level of Marker Increase/Decrease

The increase or decrease in the level of the marker may be relative to an average level of a general population. In an embodiment wherein the individual is an UHR individual for psychosis, the increase or decrease in the level of the marker may be relative to an average level of a general population of UHR individuals, for example a population of UHR individuals that is not considered to be suffering from psychosis (e.g. not yet had a FEP). In one embodiment, the increase or decrease in the level of the marker may be relative to average EU-GEI (EU Gene-Environment Interactions) study data levels, or any other UHR population study available to the skilled person. The increase or decrease may be a statistically significant increase or decrease.

References herein to an increase or decrease in the level of a given marker is intended to refer to an increase or decrease in determined marker level relative to the mean value of a non-transition group (i.e. mean value of a normal population that does not have a FEP, or preferably the mean value of UHR individuals who do not transition to FEP). The difference in marker level can be expressed as a ratio determined by dividing the determined level of the marker by the mean level of the marker for the non-transition group. Mean levels for each marker are provided herein. Therefore, the value of 1 is equal to the mean level of the marker for the non-transition group, values above 1 relate to an increase in marker level and values below 1 relate to a decrease in marker level. A fold change in level herein is expressed as this ratio. Therefore reference to at least an X fold decrease will refer to a value that is less than 1, and extends to values that are further less than 1. Similarly at least an X fold increase will refer to a value that is greater than 1, and extends to values that are further greater than 1.

In regard to the second aspect of the invention where functional outcome is determined, the ratios or increase or decrease in determined marker level refer to clinical high risk individuals with poor functional outcome [General Assessment of Functioning score <60 at 2 years] vs. clinical high risk individuals with good functional outcome [GAF>60 at 2 years]. References herein to an increase or decrease in the level of a given marker according to the second aspect is intended to refer to an increase or decrease in determined marker level relative to mean value of a normal population that does not have a FEP, or more preferably the mean value of clinical high risk individuals with good functional outcome [GAF>60 at 2 years]. Mean levels for each marker are provided herein. The difference in marker level can be expressed as a ratio determined by dividing the determined level of the marker by the mean level of the marker for the good functional outcome group.

In one embodiment, the decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, A disintegrin and metalloproteinase with thrombospondin motifs 13, Immunoglobulin lambda constant 3 and N-acetylmuramoyl-L-alanine amidase may be at least a 0.2 fold decrease. In another embodiment, the decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, A disintegrin and metalloproteinase with thrombospondin motifs 13, Immunoglobulin lambda constant 3, and N-acetylmuramoyl-L-alanine amidase may be at least a 0.24 fold decrease.

In one embodiment, the decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, A disintegrin and metalloproteinase with thrombospondin motifs 13, Immunoglobulin lambda constant 3, and N-acetylmuramoyl-L-alanine amidase may be at least a 0.99 fold decrease. In another embodiment, the decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, A disintegrin and metalloproteinase with thrombospondin motifs 13, Immunoglobulin lambda constant 3, and N-acetylmuramoyl-L-alanine amidase may be at least a 0.98 fold decrease.

In one embodiment, the decrease in the level of Alpha-2-macroglobulin may be at least about a 0.2 fold decrease. In one embodiment, the decrease in the level of Alpha-2-macroglobulin may be at least about a 0.1 fold, 0.2 fold, 0.3 fold, or 0.33 fold decrease. In one embodiment, the decrease in the level of Alpha-2-macroglobulin may be about a 0.33 fold, or more, decrease. In one embodiment, the decrease in the level of Alpha-2-macroglobulin may be about a 0.285 fold, or more, decrease. In one embodiment, the decrease in the level of Alpha-2-macroglobulin may be about a 0.4 fold, or more, decrease.

In one embodiment, the decrease in the level of Immunoglobulin heavy constant mu may be at least about a 0.1 fold decrease. In one embodiment, the decrease in the level of Immunoglobulin heavy constant mu may be at least about a 0.1 fold, 0.15 fold, 0.17 fold, 0.2 fold, 0.3 fold, 0.4 fold, or 0.41 fold decrease. In one embodiment, the decrease in the level of Immunoglobulin heavy constant mu may be about a 0.41 fold, or more, decrease. In one embodiment, the decrease in the level of Immunoglobulin heavy constant mu may be about a 0.173 fold, or more, decrease. In one embodiment, the decrease in the level of Immunoglobulin heavy constant mu may be about a 0.5 fold, or more, decrease.

In one embodiment, the decrease in the level of Phospholipid transfer protein may be at least about a 0.4 fold decrease. In one embodiment, the decrease in the level of Phospholipid transfer protein may be at least about a 0.4 fold, 0.5 fold, 0.6 fold, 0.67 or 0.7 fold decrease. In one embodiment, the decrease in the level of Phospholipid transfer protein may be about a 0.67 fold, or more, decrease. In one embodiment, the decrease in the level of Phospholipid transfer protein may be about a 0.7 fold, or more, decrease.

In one embodiment, the decrease in the level of C4b-binding protein alpha chain may be at least about a 0.5 fold decrease. In one embodiment, the decrease in the level of C4b-binding protein alpha chain may be at least about a 0.5 fold, 0.6 fold, 0.7 fold, 0.76 or 0.8 fold decrease. In one embodiment, the decrease in the level of C4b-binding protein alpha chain may be about a 0.76 fold, or more, decrease. In one embodiment, the decrease in the level of C4b-binding protein alpha chain may be about a 0.76 fold, or more, decrease. In one embodiment, the decrease in the level of C4b-binding protein alpha chain may be about a 0.8 fold, or more, decrease.

In one embodiment, the decrease in the level of Vitamin K-dependent protein S may be at least about a 0.5 fold decrease. In one embodiment, the decrease in the level of Vitamin K-dependent protein S may be about a 0.87 fold decrease. In one embodiment, the decrease in the level of Vitamin K-dependent protein S may be at least about a 0.5 fold, 0.8 fold, 0.87, or 0.9 fold decrease. In one embodiment, the decrease in the level of Vitamin K-dependent protein S may be about a 0.87 fold, or more, decrease. In one embodiment, the decrease in the level of Vitamin K-dependent protein S may be about a 0.9 fold, or more, decrease.

In one embodiment, the decrease in the level of Ficolin-3 may be at least about a 0.3 fold decrease. In one embodiment, the decrease in the level of Ficolin-3 may be at least about a 0.3 fold, 0.5 fold, 0.6 fold, 0.65 fold, 0.7, 0.8, or 0.9 fold decrease. In one embodiment, the decrease in the level of Ficolin-3 may be about a 0.7 fold, or more, decrease. In one embodiment, the decrease in the level of Ficolin-3 may be about a 0.8 fold, or more, decrease.

In one embodiment, the decrease in the level of Transthyretin may be at least about a 0.6 fold decrease. In one embodiment, the decrease in the level of Transthyretin may be at least about a 0.3 fold, 0.4 fold, 0.5 fold, 0.55 fold, 0.6 fold, 0.7 fold, 0.8 fold or 0.82 fold decrease. In one embodiment, the decrease in the level of Transthyretin may be about a 0.6 fold, or more, decrease. In one embodiment, the decrease in the level of Transthyretin may be about a 0.82 fold, or more, decrease. In one embodiment, the decrease in the level of Transthyretin may be about a 0.7 fold, or more, decrease.

In one embodiment, the decrease in the level of Plasma protease C1 inhibitor may be at least about a 0.5 fold decrease. In another embodiment, the decrease in the level of Plasma protease C1 inhibitor may be at least about a 0.5, 0.6, 0.7, 0.8, 0.85, or 0.9 fold decrease. In one embodiment, the decrease in the level of Plasma protease C1 inhibitor may be about a 0.85 fold, or more, decrease. In one embodiment, the decrease in the level of Plasma protease C1 inhibitor may be about a 0.9 fold, or more, decrease.

In one embodiment, the decrease in the level of Alpha-2-antiplasmin may be at least about a 0.6 fold decrease. In one embodiment, the decrease in the level of Alpha-2-antiplasmin may be at least about a 0.6, 0.7, 0.8, 0.9, 0.95, or 0.97 fold decrease. In one embodiment, the decrease in the level of Alpha-2-antiplasmin may be about a 0.97 fold, or more, decrease. In one embodiment, the decrease in the level of Alpha-2-antiplasmin may be about a 0.98 fold, or more, decrease.

In one embodiment, the decrease in the level of A disintegrin and metalloproteinase with thrombospondin motifs 13 (ATS13) may be at least about a 0.4 fold decrease. In another embodiment, the decrease in the level of A disintegrin and metalloproteinase with thrombospondin motifs 13 (ATS13) may be at least about a 0.4 fold, 0.6 fold, 0.7 fold, 0.8 fold, or 0.84 fold decrease. In another embodiment, the decrease in the level of A disintegrin and metalloproteinase with thrombospondin motifs 13 may be about a 0.84 fold, or more, decrease. In another embodiment, the decrease in the level of A disintegrin and metalloproteinase with thrombospondin motifs 13 may be about a 0.9 fold, or more, decrease.

In one embodiment, the decrease in the level of Immunoglobulin lambda constant 3 may be at least about a 0.1 fold decrease. In another embodiment, the decrease in the level of Immunoglobulin lambda constant 3 may be at least about a 0.1 fold, 0.11 fold, 0.4 fold, 0.5 fold, 0.6 fold, or 0.7 fold decrease. In another embodiment, the decrease in the level of Immunoglobulin lambda constant 3 may be about a 0.7 fold, or more, decrease. In another embodiment, the decrease in the level of Immunoglobulin lambda constant 3 may be about a 0.111 fold, or more, decrease. In another embodiment, the decrease in the level of Immunoglobulin lambda constant 3 may be about a 0.88 fold, or more, decrease. In another embodiment, the decrease in the level of Immunoglobulin lambda constant 3 may be about a 0.9 fold, or more, decrease.

In one embodiment, the decrease in the level of N-acetylmuramoyl-L-alanine amidase may be at least about a 0.93 fold decrease. In one embodiment, the decrease in the level of N-acetylmuramoyl-L-alanine amidase may be at least about a 0.95 fold decrease.

In one embodiment, the increase in the level of one or more markers selected from Complement component 8 alpha chain, Complement component 6, Retinol-binding protein 4, Beta-crystallin B2, Vitamin D binding protein, Inter-alpha-trypsin inhibitor heavy chain H1, Fibulin-1, Clusterin, L-lactate dehydrogenase B chain, Complement C1q subcomponent subunit C, and Alpha-crystallin A chain, coagulation factor XII, Carboxypeptidase N subunit 2, Alpha 1 anti-chymotrypsin, Plasminogen, Monocyte differentiation antigen CD14, Attractin, zinc alpha-2-glycoprotein, Extracellular matrix protein 1, Complement C1s subcomponent, Ceruloplasmin, and Complement Factor I may be at least a 1.01 fold increase. In another embodiment, the increase in the level of one or more markers selected from Complement component 8 alpha chain, Complement component 6, Retinol-binding protein 4, Beta-crystallin B2, Vitamin D binding protein, Inter-alpha-trypsin inhibitor heavy chain H1, Fibulin-1, Clusterin, L-lactate dehydrogenase B chain, Complement C1q subcomponent subunit C, Alpha-crystallin A chain, coagulation factor XII, Carboxypeptidase N subunit 2, Alpha 1 anti-chymotrypsin, Plasminogen, Monocyte differentiation antigen CD14, Attractin, zinc alpha-2-glycoprotein, Extracellular matrix protein 1, Complement C1s subcomponent, Ceruloplasmin, and Complement Factor I may be at least a 1.01 fold increase.

In one embodiment, the increase in the level of Complement component 8 alpha chain may be at least about a 1 fold increase. In another embodiment, the increase in the level of Complement component 8 alpha chain may be at least about a 1.2, 1.25, 1.3, 1.4, 1.45, 1.48 or 1.5 fold increase. In another embodiment, the increase in the level of Complement component 8 alpha chain may be about a 1.48 fold, or more, increase. In another embodiment, the increase in the level of Complement component 8 alpha chain may be about a 1.25 fold, or more, increase.

In one embodiment, the increase in the level of zinc alpha-2-glycoprotein may be at least about a 1.01 fold, or more, increase.

In one embodiment, the increase in the level of Complement component 6 may be at least about a 1.05 or 1.06, or 1.1 fold increase. In another embodiment, the increase in the level of Complement component 6 may be about a 1.06 fold, or more, increase.

In one embodiment, the increase in the level of Retinol-binding protein 4 may be at least about a 1.1, 1.2, 1.3, 1.35, 1.36, or 1.4 fold increase. In another embodiment, the increase in the level of Retinol-binding protein 4 may be about a 1.36 fold, or more, increase.

In one embodiment, the increase in the level of Beta-crystallin B2 may be at least about a 1.2, 1.4, 1.5, 1.7, 1.8 or 2 fold increase. In another embodiment, the increase in the level of Beta-crystallin B2 may be about a 1.8 fold, or more, increase.

In one embodiment, the increase in the level of Vitamin D binding protein may be at least about a 1.02, 1.2, 1.4, or 1.43 fold increase. In another embodiment, the increase in the level of Vitamin D binding protein may be about a 1.43 fold, or more, increase. In another embodiment, the increase in the level of Vitamin D binding protein may be about a 1.02 fold, or more, increase.

In one embodiment, the increase in the level of Inter-alpha-trypsin inhibitor heavy chain H1 may be at least about a 1.1, 1.15, 1.19, or 1.2 fold increase. In another embodiment, the increase in the level of Inter-alpha-trypsin inhibitor heavy chain H1 may be about a 1.19 fold, or more, increase. In another embodiment, the increase in the level of Inter-alpha-trypsin inhibitor heavy chain H1 may be about a 1.15 fold, or more, increase.

In one embodiment, the increase in the level of Fibulin-1 may be at least about a 1.2, 1.3, 1.37, 1.4, 1.5, or 1.52 fold increase. In another embodiment, the increase in the level of Fibulin-1 may be about a 1.52 fold, or more, increase. In another embodiment, the increase in the level of Fibulin-1 may be about a 1.37 fold, or more, increase.

In one embodiment, the increase in the level of Clusterin may be at least about a 1.2, 1.29, 1.3, 1.36 or 1.4 fold increase. In another embodiment, the increase in the level of Clusterin may be about a 1.29 fold, or more, increase.

In one embodiment, the increase in the level of L-lactate dehydrogenase B chain may be at least about a 1.1, 1.16 or 1.2 fold increase. In another embodiment, the increase in the level of L-lactate dehydrogenase B chain may be about a 1.16 fold, or more, increase.

In one embodiment, the increase in the level of Complement C1q subcomponent subunit C may be at least about a 1.2, 1.4, 1.5 or 1.53 fold increase. In another embodiment, the increase in the level of Complement C1q subcomponent subunit C may be about a 1.53 fold, or more, increase.

In one embodiment, the increase in the level of Carboxypeptidase N subunit 2 may be at least about a 1.06 or 1.1 fold increase. In another embodiment, the increase in the level of Carboxypeptidase N subunit 2 may be about a 1.06 fold, or more, increase.

In one embodiment, the increase in the level of alpha 1 anti-chymotrypsin may be at least about a 1.06 fold increase. In another embodiment, the increase in the level of alpha 1 anti-chymotrypsin may be about a 1.1 fold, or more, increase.

In one embodiment, the increase in the level of Plasminogen may be at least about a 1.2, 1.29, or 1.3 fold increase. In another embodiment, the increase in the level of Plasminogen may be about a 1.29 fold, or more, increase.

In one embodiment, the increase in the level of Monocyte differentiation antigen CD14 may be at least about a 1.03, 1.2, 1.3, 1.39 or 1.4 fold increase. In another embodiment, the increase in the level of Monocyte differentiation antigen CD14 may be about a 1.03 fold, or more, increase. In another embodiment, the increase in the level of Monocyte differentiation antigen CD14 may be about a 1.39 fold, or more, increase.

In one embodiment, the increase in the level of attractin may be at least about a 1.19, 1.2 or 1.3 fold increase. In another embodiment, the increase in the level of attractin may be about a 1.3 fold, or more, increase.

In one embodiment, the increase in the level of Complement factor I may be at least about a 1, 1.2, 1.23, 1.27 or 1.3 fold increase. In another embodiment, the increase in the level of Complement factor I may be about a 1.23 fold, or more, increase.

In one embodiment, the increase in the level of Alpha crystallin A chain may be at least about a 1.2, 1.3, 1.5, 1.6 or 1.63 fold increase. In another embodiment, the increase in the level of Alpha crystallin A chain may be about a 1.63 fold, or more, increase.

In one embodiment, the increase in the level of Coagulation factor XII may be at least about a 1.1 or 1.14 fold increase. In another embodiment, the increase in the level of Coagulation factor XII may be about a 1.14 fold, or more, increase.

In one embodiment, the increase in the level of Extracellular matrix protein 1 may be at least about a 1.01 fold increase.

In one embodiment, the increase in the level of Complement C1s subcomponent may be at least about a 1.06 fold increase. In another embodiment, the increase in the level of Complement C1s subcomponent may be at least about a 1.1 fold increase.

In one embodiment, the increase in the level of Antithrombin III may be at least about a 1.01 fold increase.

In accordance with the second aspect of the present invention, the increase in the level of one or more markers selected from Fetuin-B, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C may be at least a 1.1 fold increase.

In accordance with the second aspect of the present invention, the increase in the level of one or more markers selected from Fetuin-B, Inter-alpha-trypsin inhibitor heavy chain H1, Insulin-like growth factor-binding protein 3, Clusterin, Galectin-3-binding protein, Pigment epithelium-derived factor, Kininogen-1, Apolipoprotein C-III, and von Willebrand factor may be at least a 1.1 fold increase.

In one embodiment, in accordance with the second aspect of the present invention, the increase in the level of Fetuin-B may be at least about a 1.2, 1.4, 1.45 or 1.5 fold increase. In another embodiment, the increase in the level of Fetuin-B may be about a 1.45 fold, or more, increase.

In one embodiment, in accordance with the second aspect of the present invention, the increase in the level of Galectin-3-binding protein may be at least about a 1.1, 1.16, or 1.2 fold increase. In another embodiment, the increase in the level of Galectin-3-binding protein may be about a 1.16 fold, or more, increase.

In one embodiment, in accordance with the second aspect of the present invention, the increase in the level of Inter-alpha-trypsin inhibitor heavy chain H1 may be at least about a 1.2, 1.25, or 1.3 fold increase. In another embodiment, the increase in the level of Inter-alpha-trypsin inhibitor heavy chain H1 may be about a 1.25 fold, or more, increase.

In one embodiment, in accordance with the second aspect of the present invention, the increase in the level of Clusterin may be at least about a 1.1, 1.2 or 1.22 fold increase. In another embodiment, the increase in the level of Clusterin may be about a 1.22 fold, or more, increase.

In one embodiment, in accordance with the second aspect of the present invention, the increase in the level of von Willebrand factor may be at least about a 1.07 or 1.1 fold increase. In another embodiment, the increase in the level of von Willebrand factor may be about a 1.07 fold, or more, increase.

In one embodiment, in accordance with the second aspect of the present invention, the increase in the level of Insulin-like growth factor-binding protein 3 may be at least about a 1.1, 1.17 or 1.2 fold increase. In another embodiment, the increase in the level of Insulin-like growth factor-binding protein 3 may be about a 1.17 fold, or more, increase.

In one embodiment, in accordance with the second aspect of the present invention, the increase in the level of Apolipoprotein C-III may be at least about a 1.2, 1.4, 1.45 or 1.5 fold increase. In another embodiment, the increase in the level of Apolipoprotein C-III may be about a 1.45 fold, or more, increase.

In one embodiment, in accordance with the second aspect of the present invention, the increase in the level of Kininogen-1 may be at least about a 1.2, or 1.21 fold increase. In another embodiment, the increase in the level of Kininogen-1 may be about a 1.21 fold, or more, increase.

In one embodiment, in accordance with the second aspect of the present invention, the increase in the level of Pigment epithelium-derived factor may be at least about a 1.1, 1.15 or 1.2 fold increase. In another embodiment, the increase in the level of Pigment epithelium-derived factor may be about a 1.15 fold, or more, increase.

In one embodiment, in accordance with the second aspect of the present invention, the increase in the level of Complement factor H may be at least about a 1.1, 1.15 or 1.6 fold increase. In another embodiment, the increase in the level of Complement factor H may be about a 1.16 fold, or more, increase.

In one embodiment, in accordance with the second aspect of the present invention, the increase in the level of Histidine-rich glycoprotein may be at least about a 1.1, 1.2 or 1.21 fold increase. In another embodiment, the increase in the level of Histidine-rich glycoprotein may be about a 1.21 fold, or more, increase.

In one embodiment, in accordance with the second aspect of the present invention, the increase in the level of Mannose-binding protein C may be at least about a 1.1, 1.2, 1.26 or 1.3 fold increase. In another embodiment, the increase in the level of Histidine-rich glycoprotein may be about a 1.26 fold, or more, increase.

In accordance with the second aspect of the present invention, the decrease in the level of one or more markers selected from Alpha-2-macroglobulin, Phospholipid transfer protein, Immunoglobulin heavy constant mu, CD5 antigen-like, and Pyruvate kinase may be at least a 0.99 fold decrease. In accordance with the second aspect of the present invention, the decrease in the level of one or more markers selected from Alpha-2-macroglobulin, Phospholipid transfer protein, Immunoglobulin heavy constant mu, CD5 antigen-like, and Pyruvate kinase may be at least a 0.94 fold decrease.

In accordance with the second aspect of the present invention, the decrease in the level of one or more markers selected from Alpha-2-macroglobulin, Phospholipid transfer protein, Immunoglobulin heavy constant mu, CD5 antigen-like, Pyruvate kinase, and Triosephosphate isomerase may be at least a 0.3 fold decrease. In accordance with the second aspect of the present invention, the decrease in the level of one or more markers selected from Alpha-2-macroglobulin, Phospholipid transfer protein, Immunoglobulin heavy constant mu, CD5 antigen-like, Pyruvate kinase, and Triosephosphate isomerase may be at least a 0.5 fold decrease.

In one embodiment, in accordance with the second aspect of the present invention, the decrease in the level of Alpha-2-macroglobulin may be at least about a 0.1 fold decrease. In another embodiment, the decrease in the level of Alpha-2-macroglobulin may be at least about a 0.1, 0.2, 0.3, 0.4 or 0.5 fold decrease. In one embodiment, in accordance with the second aspect of the present invention, the decrease in the level of Alpha-2-macroglobulin may be at least about a 0.9 or 0.6 fold decrease. In another embodiment, the decrease in the level of Alpha-2-macroglobulin may be about a 0.5 fold, or more, decrease.

In one embodiment, in accordance with the second aspect of the present invention, the decrease in the level of Immunoglobulin heavy constant mu may be at least about a 0.2 fold decrease. In another embodiment, the decrease in the level of Immunoglobulin heavy constant mu may be at least about a 0.2, 0.3, 0.5, 0.57 or 0.6 fold decrease. In another embodiment, the decrease in the level of Immunoglobulin heavy constant mu may be at least about a 0.9, 0.7, 0.6 or 0.57 fold decrease. In another embodiment, the decrease in the level of Immunoglobulin heavy constant mu may be about a 0.57 fold, or more, decrease.

In one embodiment, in accordance with the second aspect of the present invention, the decrease in the level of Phospholipid transfer protein may be at least about a 0.2 fold decrease. In another embodiment, the decrease in the level of Phospholipid transfer protein may be at least about a 0.3, 0.5, 0.6, 0.68 or 0.7 fold decrease. In another embodiment, the decrease in the level of Phospholipid transfer protein may be at least about a 0.9, 0.8, 0.7 or 0.68 fold decrease. In another embodiment, the decrease in the level of Phospholipid transfer protein may be about a 0.68 fold, or more, decrease.

In one embodiment, in accordance with the second aspect of the present invention, the decrease in the level of CD5 antigen-like may be at least about a 0.4 fold decrease. In another embodiment, the decrease in the level of CD5 antigen-like may be at least about a 0.4, 0.8, 0.9 or 0.94 fold decrease. In another embodiment, the decrease in the level of CD5 antigen-like may be at least about a 0.99, 0.96, 0.95 or 0.94 fold decrease. In another embodiment, the decrease in the level of CD5 antigen-like may be about a 0.94 fold, or more, decrease.

In one embodiment, in accordance with the second aspect of the present invention, the decrease in the level of Pyruvate kinase may be at least about a 0.3 fold decrease. In another embodiment, the decrease in the level of Pyruvate kinase may be at least about a 0.3, 0.4, 0.5, 0.58 or 0.6 fold decrease. In another embodiment, the decrease in the level of Pyruvate kinase may be at least about a 0.9, 0.7, 0.6, or 0.58 fold decrease. In another embodiment, the decrease in the level of Pyruvate kinase may be about a 0.58 fold, or more, decrease.

In one embodiment, in accordance with the second aspect of the present invention, the decrease in the level of Triosephosphate isomerase may be at least about a 0.4 fold decrease. In another embodiment, the decrease in the level of Triosephosphate isomerase may be at least about a 0.4, 0.5, 0.6, 0.71 or 0.7 fold decrease. In another embodiment, the decrease in the level of Triosephosphate isomerase may be about a 0.71 fold, or more, decrease.

In an embodiment according to the first aspect, the level of two or more of the markers may be determined. In another embodiment according to the first aspect, the level of three or more of the markers may be determined. In another embodiment according to the first aspect, the level of four or more of the markers may be determined. In another embodiment according to the first aspect, the level of five or more of the markers may be determined. In another embodiment according to the first aspect, the level of six or more of the markers may be determined. In another embodiment according to the first aspect, the level of seven or more of the markers may be determined. In another embodiment according to the first aspect, the level of eight or more of the markers may be determined. In another embodiment according to the first aspect, the level of nine or more of the markers may be determined. In a preferred embodiment according to the first aspect, the level of ten or more of the markers may be determined. In one embodiment the level of the top five or top ten predictive markers of any of the models herein described may be determined.

In an embodiment according to the first aspect, there is provided a method of determining the likelihood of an individual transitioning to a first episode of psychosis (FEP), the method comprising:
determining the level of markers in a bodily fluid sample from the individual, wherein the markers are selected from one or more proteins of Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III,
wherein an increase in the level of one or more markers selected from Complement component 8 alpha chain, Complement component 6, Retinol-binding protein 4, Beta-crystallin B2, Vitamin D binding protein, Inter-alpha-trypsin inhibitor heavy chain H1, Fibulin-1, Clusterin, L-lactate dehydrogenase B chain, Complement C1q subcomponent subunit C, and Alpha-crystallin A chain, coagulation factor XII, Carboxypeptidase N subunit 2, Alpha 1 anti-chymotrypsin, Plasminogen, Monocyte differentiation antigen CD14, Attractin, and Complement Factor I; and/or a decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, Extracellular matrix protein 1, A disintegrin and metalloproteinase with thrombospondin motifs 13, Complement C1s subcomponent, Zinc alpha-2-glycoprotein, Immunoglobulin lambda constant 3, Ceruloplasmin and Antithrombin III, is predictive of the individual transitioning to a first episode of psychosis (FEP).

In an embodiment according to the first aspect, the marker may comprise one or more marker selected from the group comprising alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In an embodiment according to the first aspect, the marker may comprise 2, 3, 4, 5, 6, 7, 8, or 9 markers selected from the group comprising alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In an embodiment according to the first aspect, the markers may comprise alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In a preferred embodiment according to the first aspect, the marker may comprise alpha-2-macroglobulin (A2M) and 1, 2, 3, 4, 5, 6, 7, 8, or 9 markers selected from the group comprising immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In a preferred embodiment according to the first aspect, the marker may comprise alpha-2-macroglobulin (A2M) and immunoglobulin heavy constant mu (IGHM), and 1, 2, 3, 4, 5, 6, 7, or 8 markers selected from the group comprising C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In an embodiment according to the first aspect, the markers may comprise alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6).

In a particularly preferred embodiment according to the first aspect, the marker may comprise Alpha-2-macroglobulin. A decrease in the level of Alpha-2-macroglobulin may be observed. In another preferred embodiment according to the first aspect, the marker may comprise Immunoglobulin heavy constant mu. A decrease in the level of Immunoglobulin heavy constant mu may be observed. In another preferred embodiment according to the first aspect, the markers may comprise Alpha-2-macroglobulin and Immunoglobulin heavy constant mu. A decrease in the level of both Alpha-2-macroglobulin and Immunoglobulin heavy constant mu may be observed.

Advantageously, the use of Alpha-2-macroglobulin and Immunoglobulin heavy constant mu alone or in combination, can be particularly predictive of a transition to FEP, for example for an UHR individual.

In another embodiment according to the first aspect, the markers may comprise Alpha-2-macroglobulin, Immunoglobulin heavy constant mu, and C4b-binding protein alpha chain. In another embodiment according to the first aspect, the markers may comprise Alpha-2-macroglobulin, Immunoglobulin heavy constant mu, C4b-binding protein alpha chain, and Phospholipid transfer protein.

In another embodiment according to the first aspect, the markers may comprise Alpha-2-macroglobulin, Immunoglobulin heavy constant mu, C4b-binding protein alpha chain, Phospholipid transfer protein and Transthyretin. In an alternative embodiment according to the first aspect, the markers may comprise Alpha-2-macroglobulin, Immunoglobulin heavy constant mu, C4b-binding protein alpha chain, vitamin K dependent protein S, and fibulin-1.

Advantageously, the use of the top five biomarkers of Alpha-2-macroglobulin, Immunoglobulin heavy constant mu, C4b-binding protein alpha chain, Phospholipid transfer protein and Transthyretin, or the alternative top five biomarkers of Alpha-2-macroglobulin, Immunoglobulin heavy constant mu, C4b-binding protein alpha chain, vitamin K dependent protein S, and fibulin-1, can be particularly predictive of a transition to FEP, for example for an UHR individual.

In another embodiment according to the first aspect, the markers may comprise Alpha-2-macroglobulin, Immunoglobulin heavy constant mu, C4b-binding protein alpha chain, Phospholipid transfer protein, Transthyretin and Vitamin D binding protein. In another embodiment according to the first aspect, the markers may comprise Alpha-2-macroglobulin, Immunoglobulin heavy constant mu, C4b-binding protein alpha chain, Phospholipid transfer protein, Transthyretin, Vitamin D binding protein and Beta-crystallin B2. In another embodiment according to the first aspect, the markers may comprise Alpha-2-macroglobulin, Immunoglobulin heavy constant mu, C4b-binding protein alpha chain, Phospholipid transfer protein, Transthyretin, Vitamin D binding protein, Beta-crystallin B2 and Vitamin K-dependent protein S. In another embodiment according to the first aspect, the markers may comprise Alpha-2-macroglobulin, Immunoglobulin heavy constant mu, C4b-binding protein alpha chain, Phospholipid transfer protein, Transthyretin, Vitamin D binding protein, Beta-crystallin B2, Vitamin K-dependent protein S and Coagulation factor XII. In another embodiment according to the first aspect, the markers may comprise Alpha-2-macroglobulin, Immunoglobulin heavy constant mu, C4b-binding protein alpha chain, Phospholipid transfer protein, Transthyretin, Vitamin D binding protein, Beta-crystallin B2, Vitamin K-dependent protein S, Coagulation factor XII and Clusterin.

In an embodiment according to the first aspect, the marker may comprise one or more marker selected from the group comprising alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin. In an embodiment according to the first aspect, the marker may comprise 2, 3, 4, 5, 6, 7, 8, or 9 markers selected from the group comprising alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin. In an embodiment according to the first aspect, the markers may comprise alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin.

In an embodiment according to the second aspect, the level of two or more of the markers may be determined. In another embodiment according to the second aspect, the level of three or more of the markers may be determined. In another embodiment according to the second aspect, the level of four or more of the markers may be determined. In another embodiment according to the second aspect, the level of five or more of the markers may be determined. In another embodiment according to the second aspect, the level of six or more of the markers may be determined. In another embodiment according to the second aspect, the level of seven or more of the markers may be determined. In another embodiment according to the second aspect, the level of eight or more of the markers may be determined. In another embodiment according to the second aspect, the level of nine or more of the markers may be determined. In a preferred embodiment according to the second aspect, the level of ten or more of the markers may be determined.

In an embodiment according to the second aspect, the marker may comprise Alpha-2-macroglobulin. In an embodiment according to the second aspect, the marker may comprise Immunoglobulin heavy constant mu. In an embodiment according to the second aspect, the marker may comprise Fetuin-B. In an embodiment according to the second aspect, the marker may comprise Phospholipid transfer protein. In an embodiment according to the second aspect, the marker may comprise CD5 antigen-like. In an embodiment according to the second aspect, the marker may comprise Pyruvate kinase. In an embodiment according to the second aspect, the marker may comprise Galectin-3-binding protein. In an embodiment according to the second aspect, the marker may comprise Inter-alpha-trypsin inhibitor heavy chain H1. In an embodiment according to the second aspect, the marker may comprise von Willebrand factor. In an embodiment according to the second aspect, the marker may comprise Clusterin. In an embodiment according to the second aspect, the marker may comprise Insulin-like growth factor-binding protein 3. In an embodiment according to the second aspect, the marker may comprise Pigment epithelium-derived factor. In an embodiment according to the second aspect, the marker may comprise Triosephosphate isomerase. In an embodiment according to the second aspect, the marker may comprise Apolipoprotein C-III. In an embodiment according to the second aspect, the marker may comprise Kininogen-1.

In one embodiment according to the second aspect, the markers may comprise Alpha-2-macroglobulin and any one or more proteins selected from Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, and Pigment epithelium-derived factor. In another embodiment according to the second aspect, the markers may comprise Alpha-2-macroglobulin, Phospholipid transfer protein and any one or more proteins selected from, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, and Pigment epithelium-derived factor. In another embodiment according to the second aspect, the markers may comprise Alpha-2-macroglobulin, Phospholipid transfer protein, Immunoglobulin heavy constant mu and any one or more proteins selected from CD5 antigen-like, Fetuin-B, Inter-alpha-trypsin inhibitor heavy chain H1, Pyruvate kinase, Insulin-like growth factor-binding protein 3, Clusterin, Galectin-3-binding protein, and Pigment epithelium-derived factor. In another embodiment according to the second aspect, the markers may comprise Alpha-2-macroglobulin, Phospholipid transfer protein, Immunoglobulin heavy constant mu, CD5 antigen-like, and any one or more proteins selected from Fetuin-B, Inter-alpha-trypsin inhibitor heavy chain H1, Pyruvate kinase, Insulin-like growth factor-binding protein 3, Clusterin, Galectin-3-binding protein, and Pigment epithelium-derived factor. In another embodiment according to the second aspect, the markers may comprise Alpha-2-macroglobulin, Phospholipid transfer protein, Immunoglobulin heavy constant mu, CD5 antigen-like, Fetuin-B, and any one or more proteins selected from Inter-alpha-trypsin inhibitor heavy chain H1, Pyruvate kinase, Insulin-like growth factor-binding protein 3, Clusterin, Galectin-3-binding protein, and Pigment epithelium-derived factor. In another embodiment according to the second aspect, the markers may comprise Alpha-2-macroglobulin, Phospholipid transfer protein, Immunoglobulin heavy constant mu, CD5 antigen-like, Fetuin-B, Inter-alpha-trypsin inhibitor heavy chain H1 and any one or more proteins selected from Pyruvate kinase, Insulin-like growth factor-binding protein 3, Clusterin, Galectin-3-binding protein, and Pigment epithelium-derived factor. In another embodiment according to the second aspect, the markers may comprise Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, and any one or more proteins selected from, Clusterin, Galectin-3-binding protein, and Pigment epithelium-derived factor. In another embodiment according to the second aspect, the markers may comprise Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, and any one or more proteins selected from Galectin-3-binding protein, and Pigment epithelium-derived factor. In another embodiment according to the second aspect, the markers may comprise Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, and Pigment epithelium-derived factor.

In another embodiment according to the second aspect, the markers may comprise 2, 3, 4, 5, 6, 7, 8, 9, 10, or more of the markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C. In another embodiment according to the second aspect, the markers may comprise Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3. In another embodiment according to the second aspect, the markers may comprise 2, 3, 4, 5, 6, 7, 8, 9 or all of the markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3.

### Interventions/Actions

The methods of the invention may be used, for example, for any one or more of the following: to advise on the prognosis for an individual with UHR for psychosis; to advise on treatment options for an individual with UHR for psychosis, for example to avoid psychosis or avoid a FEP, or avoid developing functional disability. The presence, or level, of the markers may be used to stratify patients. This stratification may be used to decide the appropriate treatment.

Information regarding the UHR status of a subject may be relayed to a third party, such as a doctor, other medical professional, pharmacist or other interested party. This information may be relayed digitally, for example via email, SMS or other digital means.

The method may further comprise selecting the individual for therapeutic intervention and/or a follow-up check, if the individual is predicted to transition to a first episode of psychosis (FEP) and/or develop a functional disability. In another embodiment, the method further comprises administering a therapeutic or preventative medication to the individual, if the individual is predicted to transition to a first episode of psychosis (FEP) and/or develop a functional disability. The medication may comprise an anti-psychotic medication and/or an antidepressant medication. Additionally or alternatively, if an individual is not predicted to transition to a FEP and/or develop a functional disability, the individual may not be selected for therapeutic intervention and/or a follow-up check. In another embodiment, if an individual is not predicted to transition to a FEP and/or develop a functional disability, the individual may be selected for an alternative non-anti-psychotic therapy.

If the individual is not predicted to transition to a first episode of psychosis (FEP) and/or develop a functional disability, the individual may be provided with one or more of psychotherapy, family therapy and nutritional therapy. If the individual is predicted to transition to a first episode of psychosis (FEP) and/or develop a functional disability, the individual may be provided with one or more of medication, psychotherapy, family therapy and nutritional therapy. If the individual is predicted to transition to a first episode of psychosis (FEP) and/or develop a functional disability, the individual may be subject to increased monitoring of their psychosis state and/or their functional ability.

### Medication

The medication may comprise an anti-psychotic medication. Additionally or alternatively the medication may comprise an antidepressant medication.

In one embodiment, the medication comprises one or more of a dopamine blocker/inhibitor, a beta-blocker, omega-3 fatty acids, D-serine, and glycine.

The anti-psychotic medication may comprise a medication selected from the group comprising amisulpride, aripiprazole, asenapine, benperidol, chlorpromazine, clozapine, flupentixol, fluphenazine, haloperidol, levomepromazine, lurasidone, olanzapine, paliperidone, pericyazine, pimozide, pipotiazine, prochlorperazine, promazine, quetiapine, risperidone, sulpiride, trifluoperazine, and zuclopenthixol; analogues, derivatives or salts thereof; and combinations thereof.

The antidepressant may comprise a medication selected from the group comprising a SSRI (selective serotonin reuptake inhibitor), a SNRI (serotonin and norepinephrine reuptake inhibitor), and a tricyclic antidepressant.

The skilled person will recognise that the medication can be administered, or arranged to be administered, at an appropriate dose via the appropriate administration route. In one embodiment, the medication may comprise or consist of a therapeutically or prophylactically effective amount of the medication.

### Marker Level Determination

In one embodiment, the presence, absence, or level of a marker may be determined by any suitable assay. The skilled person will recognise there are a number of methods and technologies available to determine the presence and/or level of a marker, such as a protein marker.

Determining the level of a marker may comprise quantifying the presence of the marker in the sample. The level of a marker in the sample may be compared relative to a control sample, or a predetermined standard level.

Determining the level of a marker may comprise binding a marker with one or more probes. The method may further comprise detecting the act of binding of the probe(s) to the marker, or detecting the level of bound probe-marker complexes.

In one embodiment, determining the level of a marker may comprise conducting an enzyme-linked immunosorbent assay (ELISA). In particular an ELISA may be used to determine the level of one or more markers in the sample. The ELISA may comprise multi-analyte ELISA. The ELISA may be direct or indirect.

In another embodiment, the level of a marker may be determined by western blot.

In another embodiment, the level of a marker may be determined by a Proximity Extension Assay (PEA) (e.g. by Olink Proteomics™). For example, PEA may comprise the provision of a matched pair of proximity probes, for example comprising a pair of antibodies, or fragments thereof, arranged to bind the targeted marker, and both linked to respective oligonucleotides arranged to hybridise to each other. As a result of the proximity probes binding to the same targeted marker, the probes can come in close proximity and hybridize to each other. The addition of a DNA polymerase can lead to an extension of the hybridizing oligo, bound to one of the probes, creating a DNA amplicon that can subsequently be detected and quantified, for example by quantitative real-time PCR.

In another embodiment, the level of a marker may be determined by detecting the marker directly, for example by mass-spectrometry. The mass-spectrometry may comprise liquid chromatography mass spectrometry. The mass-spectrometry may comprise multiple reaction monitoring (MRM) (also known as selection reaction monitoring).

### Probes

The probe (according to any aspect of the invention) may be a binding agent that is capable of specific/selective binding to a protein marker of interest. The binding agent may comprise or consist of a polypeptide and/or nucleic acid, such as DNA. The probe may comprise or consist of an antibody, an antibody variant or memetic, or a binding-fragment thereof. In another embodiment, the probe may comprise or consist of an aptamer. In one embodiment, the probe for a given marker is a polyclonal or monoclonal antibody, or fragment thereof. The antibody, or fragment thereof, may be of any mammalian species, such as human, simian or rabbit.

In one embodiment, the probes are immobilised on a substrate. One or more, or all of the probes may be anchored to a surface, such as the surface of a solid substrate. The solid substrate may be a plate, such as a microwell plate. In another embodiment, the solid substrate may be a particle, such as a nano- or micro-particle. In one embodiment the solid substrate is a bead.

In one embodiment, the probe may comprise a tag identification and/or capture. The tag may comprise a fluorescent molecule, or an enzyme. In another embodiment, the probe may be radiolabelled.

Where ELISA, or similar assay, is used, the probe may be a primary antibody for binding to the target, and a secondary tagged-antibody probe may be provided for binding to the primary antibody or the marker for detection.

### The Sample

The bodily fluid sample may comprise extracellular fluid. The bodily fluid sample may comprise or consist of a bodily fluid selected from blood, serum, plasma, cerebral spinal fluid, mucus, saliva, and urine. In another embodiment, the bodily fluid sample may comprise or consist of a bodily fluid selected from blood, serum, plasma, and cerebral spinal fluid. In one embodiment, the sample comprises or consists of a plasma sample. The sample may be taken/obtained from the individual in the method of the invention. Alternatively, the sample may be provided (previously obtained, for example by a third party). The sample may be fresh, such as less than 1 day from withdrawal. Alternatively, the sample may be a stored sample, for example that has been frozen or refrigerated.

Some or all of the steps of the method(s) of the invention may be carried out in vitro.

### Other Aspects

According to another aspect of the present invention, there is provided a composition comprising a plurality (e.g. two or more) of probes capable of binding to protein markers in a bodily fluid sample, wherein the protein markers comprise two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

In one embodiment, the composition may comprise a plurality (e.g. two or more) of probes capable of binding to protein markers in a bodily fluid sample, wherein the protein markers comprise two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In one embodiment, a probe may be provided for at least 3 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In one embodiment, a probe may be provided for at least 3 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

In another embodiment, a probe may be provided for at least 5 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In another embodiment, a probe may be provided for at least 5 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

In another embodiment, a probe may be provided for at least 8 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In another embodiment, a probe may be provided for at least 8 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

In another embodiment, a probe may be provided for at least 10 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In another embodiment, a probe may be provided for at least 10 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In another embodiment, a probe may be provided for at least 15 of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

In another embodiment, a probe may be provided for each of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In another embodiment, a probe may be provided for each of the protein markers selected from Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

In another embodiment, a probe may be provided for one or more, or all, of the protein markers selected from alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In another embodiment, a probe may be provided for one or more, or all, of the protein markers selected from alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin. In another embodiment, a probe may be provided for one or more, or all, of the protein markers selected from Alpha-2-macroglobulin, Immunoglobulin heavy constant mu, C4b-binding protein alpha chain, Phospholipid transfer protein, Transthyretin, Vitamin D binding protein, Beta-crystallin B2, Vitamin K-dependent protein S and Coagulation factor XII. In another embodiment according to the first aspect, the markers may comprise Alpha-2-macroglobulin, Immunoglobulin heavy constant mu, C4b-binding protein alpha chain, Phospholipid transfer protein, Transthyretin, Vitamin D binding protein, Beta-crystallin B2, Vitamin K-dependent protein S, Coagulation factor XII and Clusterin.

In a particularly preferred embodiment a probe may be provided for Alpha-2-macroglobulin. In another preferred embodiment according a probe may be provided for Immunoglobulin heavy constant mu. In another preferred embodiment probes may be provided for Alpha-2-macroglobulin and Immunoglobulin heavy constant mu.

In another embodiment probes may be provided for Alpha-2-macroglobulin, Immunoglobulin heavy constant mu, and C4b-binding protein alpha chain. In another embodiment probes may be provided for Alpha-2-macroglobulin, Immunoglobulin heavy constant mu, C4b-binding protein alpha chain, and Phospholipid transfer protein.

In another embodiment probes may be provided for Alpha-2-macroglobulin, Immunoglobulin heavy constant mu, C4b-binding protein alpha chain, Phospholipid transfer protein and Transthyretin. In an alternative embodiment probes may be provided for Alpha-2-macroglobulin, Immunoglobulin heavy constant mu, C4b-binding protein alpha chain, vitamin K dependent protein S, and fibulin-1.

According to another aspect of the present invention, there is provided a composition comprising a plurality (e.g. two or more) of probes capable of binding to protein markers in a bodily fluid sample, wherein the protein markers comprise two or more proteins selected from the group comprising Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor.

According to another aspect of the present invention, there is provided a composition comprising a plurality (e.g. two or more) of probes capable of binding to protein markers in a bodily fluid sample, wherein the protein markers comprise two or more proteins selected from the group comprising Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C.

In one embodiment, a probe may be provided for at least 3 of the protein markers selected from Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor. In another embodiment, a probe may be provided for at least 5 of the protein markers selected from Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor. In another embodiment, a probe may be provided for at least 8 of the protein markers selected from Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor. In another embodiment, a probe may be provided for at least 10 of the protein markers selected from Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor. In another embodiment, a probe may be provided for at least 15 of the protein markers selected from Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor. In another embodiment, a probe may be provided for each of the protein markers selected from Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor.

In one embodiment, a probe may be provided for at least 3 of the protein markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C. In another embodiment, a probe may be provided for at least 5 of the protein markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C. In another embodiment, a probe may be provided for at least 8 of the protein markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C. In another embodiment, a probe may be provided for at least 10 of the protein markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C. In another embodiment, a probe may be provided for at least 12 of the protein markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C. In another embodiment, a probe may be provided for each of the protein markers selected from Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C.

According to another aspect of the present invention, there is provided a composition comprising a plurality (e.g. two or more) of probes capable of binding to protein markers in a bodily fluid sample, wherein the protein markers comprise two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III, CD5 antigen-like, Fetuin-B, Pyruvate kinase, Insulin-like growth factor-binding protein 3, Galectin-3-binding protein, Pigment epithelium-derived factor, Kininogen-1, Triosephosphate isomerase, Apolipoprotein C-III, and von Willebrand factor.

In another embodiment, the protein markers comprise three, four, five, six, seven, eight, nine, ten, or more, proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III, CD5 antigen-like, Fetuin-B, Pyruvate kinase, Insulin-like growth factor-binding protein 3, Galectin-3-binding protein, Pigment epithelium-derived factor, Kininogen-1, Triosephosphate isomerase, Apolipoprotein C-III, and von Willebrand factor. In another embodiment 20 or more proteins may be selected.

According to another aspect of the present invention, there is provided a composition comprising a plurality (e.g. two or more) of probes capable of binding to protein markers in a bodily fluid sample, wherein the protein markers comprise two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase, Immunoglobulin heavy constant mu, Fetuin-B, CD5 antigen-like, Pyruvate kinase, Complement factor H, Pigment epithelium-derived factor, Insulin-like growth factor-binding protein 3, Histidine-rich glycoprotein, Galectin-3-binding protein, and Mannose-binding protein C.

In another embodiment, the protein markers comprise three, four, five, six, seven, eight, nine, ten, fifteen, twenty or more, proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase, Immunoglobulin heavy constant mu, Fetuin-B, CD5 antigen-like, Pyruvate kinase, Complement factor H, Pigment epithelium-derived factor, Insulin-like growth factor-binding protein 3, Histidine-rich glycoprotein, Galectin-3-binding protein, and Mannose-binding protein C. In one embodiment, 20 or more proteins may be selected.

The composition(s) according to the invention may comprise a probe species for each marker to be bound (i.e. each protein may have its own corresponding probe arranged to bind thereto). The plurality of probes may be different to each other (i.e. may be structurally different and bind different proteins/markers or different non-competing epitopes on the same protein/marker).

The probes may be provided as a panel of probes anchored to a surface.

The probes may be capable of selectively binding to three, four, five, six, seven, eight, nine, ten, or more, of the markers.

According to another aspect of the present invention, there is provided a method of detecting the level of two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase, in a bodily fluid sample; and optionally the bodily fluid sample may be of an ultra-high risk (UHR) individual for psychosis.

According to another aspect of the present invention, there is provided a method of detecting the level of two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III, in a bodily fluid sample; and optionally the bodily fluid sample may be of an ultra-high risk (UHR) individual for psychosis.

The method may comprise the detection of a complex formed between two or more probes and two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

The method may comprise the detection of a complex formed between two or more probes and two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

According to another aspect of the present invention, there is provided a method of detecting the level of two or more proteins selected from the group comprising Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor, in a bodily fluid sample from an individual; and optionally the bodily fluid sample may be of a ultra-high risk (UHR) individual for psychosis.

According to another aspect of the present invention, there is provided a method of detecting the level of two or more proteins selected from the group comprising Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C, in a bodily fluid sample from an individual; and optionally the bodily fluid sample may be of a ultra-high risk (UHR) individual for psychosis.

The method may comprise the detection of a complex formed between two or more probes and two or more proteins selected from the group comprising Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor.

The method may comprise the detection of a complex formed between two or more probes and two or more proteins selected from the group comprising Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C.

In one embodiment according to any of the aspects of the invention, at least 3, 4, 5, 6, 7, 8, 9, 10, 12, 15, or all, of the listed proteins are detected, or attempted to be detected. In one embodiment according to any of the aspects of the invention, the level of at least 3, 4, 5, 6, 7, 8, 9, 10, 12, 15, or all, of the listed proteins is determined. In one embodiment according to any of the aspects of the invention, at least 3, 4, 5, 6, 7, 8, 9, 10, 12, or all, of the listed proteins are detected, or attempted to be detected. In one embodiment according to any of the aspects of the invention, the level of at least 3, 4, 5, 6, 7, 8, 9, 10, 15, or all, of the listed proteins is determined.

According to another aspect of the present invention, there is provided a method for treating an individual to prevent a transition to a FEP, the method comprising the steps of:
determining whether the individual is predicted to transition to a FEP by:
   obtaining or having obtained a sample from the individual; and performing or having performed the method according to the invention herein to determine if the individual is predicted to transition to a FEP; and
if the individual is predicted to transition to a FEP, then administering medication as described herein to the individual.

According to another aspect of the present invention, there is provided a method for treating an individual to prevent a transition to a FEP, the method comprising the steps of:
receiving results of a test performed according to the method of the invention herein to determine if the individual is predicted to transition to a FEP; and
if the individual is predicted to transition to a FEP, then administering medication as described herein to the individual.

According to another aspect of the present invention, there is provided a method for treating an individual to prevent a functional disability following a FEP, the method comprising the steps of:
determining whether the individual is predicted to develop a functional disability by:
   obtaining or having obtained a sample from the individual; and performing or having performed the method according to the invention herein to determine if the individual is predicted to develop a functional disability; and
if the individual is predicted to develop a functional disability, then administering medication as described herein to the individual.

According to another aspect of the present invention, there is provided a method for treating an individual to prevent the development of a functional disability following a FEP, the method comprising the steps of:
receiving results of a test performed according to the method of the invention herein to determine if the individual is predicted to develop a functional disability; and
if the individual is predicted to develop a functional disability, then administering medication as described herein to the individual.

According to another aspect of the present invention, there is provided the use of one or more proteins as a predictive biomarker for an individual to transition to a FEP, wherein the protein is selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

According to another aspect of the present invention, there is provided the use of one or more proteins as a predictive biomarker for an individual to transition to a FEP, wherein the protein is selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin and Antithrombin III.

In one embodiment according to the method or use of the invention herein, the protein is selected from the group comprising alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In another embodiment according to the method or use of the invention herein, the protein is 2, 3, 4, 5, 6, 8, or 9 proteins selected from the group comprising alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6). In another embodiment according to the method or use of the invention herein, the proteins comprise alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6).

In one embodiment according to the method or use of the invention herein, the protein is selected from the group comprising alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin. In another embodiment according to the method or use of the invention herein, the protein is 2, 3, 4, 5, 6, 8, or 9 proteins selected from the group comprising alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin. In another embodiment according to the method or use of the invention herein, the proteins comprise alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin.

According to another aspect of the present invention, there is provided the use of one or more proteins as a predictive biomarker for predicting the likelihood of a functional disability outcome for an individual following a FEP, wherein the protein is selected from the group comprising Alpha-2-macroglobulin, Phospholipid transfer protein, Immunoglobulin heavy constant mu, CD5 antigen-like, Fetuin-B, Inter-alpha-trypsin inhibitor heavy chain H1, Pyruvate kinase, Insulin-like growth factor-binding protein 3, Clusterin, Galectin-3-binding protein, Pigment epithelium-derived factor, Kininogen-1, Triosephosphate isomerase, Apolipoprotein C-III, and von Willebrand factor.

According to another aspect of the present invention, there is provided the use of one or more proteins as a predictive biomarker for predicting the likelihood of a functional disability outcome for an individual following a FEP, wherein the protein is selected from the group comprising Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C.

The use may comprise determining the levels of the protein(s) in a bodily fluid sample from the individual. The individual may be an UHR individual for psychosis.

In a particularly preferred embodiment, the use may be of Alpha-2-macroglobulin, and/or Immunoglobulin heavy constant mu. In another embodiment the use may be of Alpha-2-macroglobulin, Immunoglobulin heavy constant mu, and C4b-binding protein alpha chain. In another embodiment the use may be of Alpha-2-macroglobulin, Immunoglobulin heavy constant mu, C4b-binding protein alpha chain, and Phospholipid transfer protein. In another embodiment the use may be of Alpha-2-macroglobulin, Immunoglobulin heavy constant mu, C4b-binding protein alpha chain, Phospholipid transfer protein and Transthyretin. In an alternative embodiment the use may be of Alpha-2-macroglobulin, Immunoglobulin heavy constant mu, C4b-binding protein alpha chain, vitamin K dependent protein S, and fibulin-1.

### Definitions

The term ‘psychosis’ is understood to mean a mental health condition that causes people to perceive or interpret things differently from those around them. This might involve hallucinations or delusions.

The ‘ultra high risk’ (UHR) state for psychosis may also be known as “at risk mental state” or “clinical high risk for psychosis”. In a preferred embodiment, a UHR individual may be determined by the Comprehensive Assessment of At-Risk Mental States²⁰ (CAARMS), which is used to assess subclinical psychotic symptoms. The CAARMS uses a semi-structured interview to determine whether subjects are not at risk, meet UHR criteria, or have psychotic disorder. Fusar-Poli et al. (JAMA Psychiatry. 2013 January; 70(1): 107-120., which is incorporated herein by reference) describes the UHR state and its method of assessment. Identification of UHR state for an individual requires the presence of one or more of the following: attenuated psychotic symptoms (APS), brief limited intermittent psychotic episode (BLIP), and trait vulnerability plus a marked decline in psychosocial functioning (genetic risk and deterioration syndrome [GRD]) and unspecified prodromal symptoms (UPS). Alternative assessment instruments to determine a UHR individual may also be used, such as any one or more of the assessment instruments of Structured Interview for Prodromal Syndromes/Scale of Prodromal Symptoms (SIPS/SOPS), SPIA-A (Schizophrenia Proneness Instrument, adult version), SPI-CY (Schizophrenia Proneness Instrument, child and youth version, and/or BSIP (Basel Screening Instrument for psychosis), in accordance with Addington et al (2012. Schizophr. Res., 142 (2012), pp. 77-82), McGlashan et al. (2001. , eds. Early Intervention in Psychotic Disorders. Dordrecht, The Netherlands: Kluwer Academic Publishers. pp. 135-149.), Schultze-Lutter et al. (2012. Schizophrenia Proneness Instrument—Child and Youth Version, Extended English Translation (SPI-CY EET). Giovanni Fioriti Editore s.l.r, Rome), Riecher-Rossler et al (2008. Fortschritte der Neurologie-Psychiatrie [1 Apr. 2008, 76(4):207-216]), which are herein incorporated by reference.

The term ‘psychotic experience’ is intended to include an individual having one or more of auditory hallucinations, visual hallucinations, and beliefs about being spied on, and where such experiences are not attributable to the effects of sleep or fever that had occurred at least once per month over the previous 6 months and either caused severe distress, had a markedly negative impact on social or occupational function, or led to help seeking. Criteria for a psychotic experience is described in Zammit et al. (Am J Psychiatry. 2013; 170(7):742-750., which is incorporated herein by reference).

The term ‘first episode psychosis’ (FEP) refers to the first time an individual experiences psychotic symptoms or a psychotic episode. The FEP may be the first episode of a psychotic disorder, whereby the psychotic symptoms surpass clinical thresholds in terms of intensity, frequency, duration and functional impact, and may necessitate clinical management or treatment. In one embodiment the FEP may be as defined by the CAARMS criteria. A person skilled in the art, such as a clinician, will be capable of identifying a FEP. In one embodiment, a FEP may be determined by the criteria described by Yung et al. (Australian and New Zealand Journal of Psychiatry 2005; 39:964-971, which is herein incorporated by reference), whereby a Psychotic disorder threshold is reached, and comprises
a severity scale score of 6 on disorders of thought content subscale, 5 or 6 on perceptual abnormalities subscale and/or 6 on disorganized speech subscale of the CAARMS;
a frequency scale score of greater than or equal to 4 on disorders of thought content, perceptual abnormalities and/or disorganized speech subscale; and
psychotic symptoms present for longer than 1 week.

“Schizophrenia” is a psychiatric disorder generally characterized by continuous or relapsing episodes of psychosis and a diagnosis or prediction of schizophrenia and it is recognized by the skilled person in the art that it is clinically distinct from a diagnosis or prediction of a ‘first episode psychosis’ (FEP). The invention herein may not be a method to predict the likelihood of an individual to have, or develop, schizophrenia, or a method of diagnosis of schizophrenia, or predisposition to schizophrenia. The invention herein may not be used to predict the likelihood of an individual to have, or develop, schizophrenia. In one embodiment, schizophrenia is identified according to the classifications of “The ICD-10 Classification of Mental and Behavioural Disorders”, Diagnostic criteria for research (World Health Organisation. ICD-10: international statistical classification of diseases and related health problems: tenth revision. 2nd ed. Geneva: World Health Organization; 2004., which is herein incorporated by reference). In particular, referring to the ICD-10 Classification of Mental and Behavioural Disorders schizophrenia can be characterised by the general criteria for paranoid, hebephrenic, catatonic and undifferentiated type of Schizophrenia: G1. Either at least one of the syndromes, symptoms and signs listed below under (1), or at least two of the symptoms and signs listed under (2), should be present for most of the time during an episode of psychotic illness lasting for at least one month (or at some time during most of the days). (1) At least one of the following: a) Thought echo, thought insertion or withdrawal, or thought broadcasting. b) Delusions of control, influence or passivity, clearly referred to body or limb movements or specific thoughts, actions, or sensations; delusional perception. c) Hallucinatory voices giving a running commentary on the patient's behaviour, or discussing him between themselves, or other types of hallucinatory voices coming from some part of the body. d) Persistent delusions of other kinds that are culturally inappropriate and completely impossible (e.g. being able to control the weather, or being in communication with aliens from another world). (2) or at least two of the following: e) Persistent hallucinations in any modality, when occurring every day for at least one month, when accompanied by delusions (which may be fleeting or half-formed) without clear affective content, or when accompanied by persistent over-valued ideas. f) Neologisms, breaks or interpolations in the train of thought, resulting in incoherence or irrelevant speech. g) Catatonic behaviour, such as excitement, posturing or waxy flexibility, negativism, mutism and stupor. h) “Negative” symptoms such as marked apathy, paucity of speech, and blunting or incongruity of emotional responses (it must be clear that these are not due to depression or to neuroleptic medication). G2. Most commonly used exclusion criteria: If the patient also meets criteria for manic episode (F30) or depressive episode (F32), the criteria listed under G1.1 and G1.2 above must have been met before the disturbance of mood developed. G3. The disorder is not attributable to organic brain disease (in the sense of FO), or to alcohol- or drug-related intoxication, dependence or withdrawal.

The ‘functional disability’ and ‘functional outcome’ may be determined by the General Assessment of Functioning (GAF) score for functional disability, recorded at assessment closest to 2 years from baseline. The GAF assessment is described in Aas (Ann Gen Psychiatry. 2010; 9:20.) and Goldman et al. (Am J Psychiatry. 1992; 149(9):1148-1156.), which are herein incorporated by reference. GAF symptoms are also listed in eTable 1. Disability is rated independently of symptoms and therefore an appropriate measure of functioning distinct from transition status. For use as a classificatory target variable the score was dichotomised into ‘poor functioning’ (<60 points; i.e. moderate or severe impairment) or ‘good functioning’ (>60 points; i.e. mild or no impairment).

By “antibody” we include substantially intact antibody molecules, as well as chimeric antibodies, human antibodies, humanised antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments, antibody mimetics, and derivatives of the same. In particular, the term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It has been shown that fragments of a whole antibody can perform the function of binding antigens. Examples of binding fragments of the invention are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers and; (ix) “diabodies”, multivalent or multispecific fragments constructed by gene fusion.

The markers listed herein may include variants of the markers, for example variants having natural mutations/polymorphisms in a population. It is understood that reference to protein or nucleic acid “variants”, is understood to mean a protein or nucleic acid sequence that has at least 70%, 80%, 90%, 95%, 98%, 99%, 99.9% identity with the sequence of the fore mentioned protein or nucleic acid. The percentage identity may be calculated under standard NCBI blast p/n alignment parameters. “Variants” may also include truncations of a protein or nucleic acid sequence. Variants may include biomarker listed herein comprising the same sequence, but comprising or consisting of 1, 2, 3, 4, 5, 6, 7, 8, 9, or 10, or more modifications, such as substitutions, deletions, additions of nucleotides or bases. Variants may also comprise redundant/degenerate codon variations.

A ‘therapeutically effective amount’, or ‘effective amount’, or ‘therapeutically effective’, as used herein, refers to that amount which provides a therapeutic or preventative effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the individual. Alternatively, a therapeutically effective amount is sufficient to cause an improvement in a clinically significant condition in an individual. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical worker based on patient/individual characteristics, such as age, weight, sex, condition, complications, other diseases, etc., as is well known in the art.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

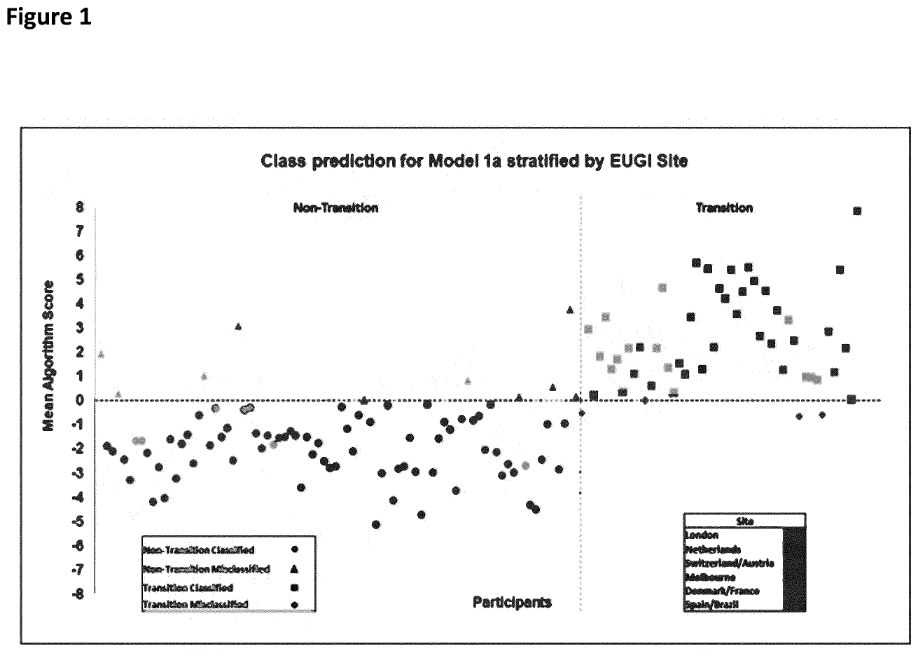

FIG. 1: Class prediction for Model 1a stratified by EU-GEI site.

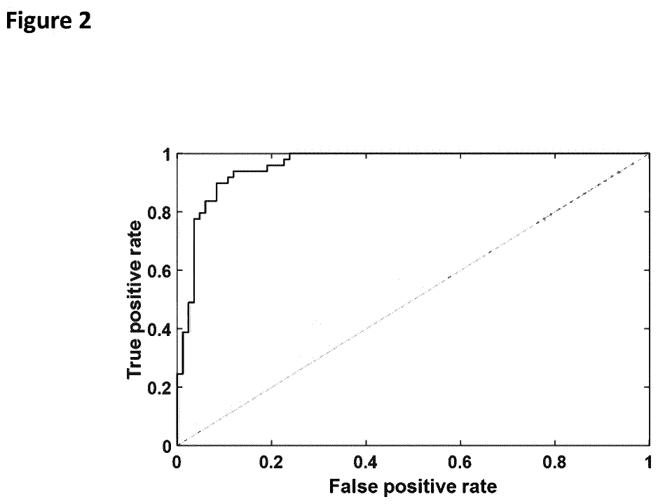

FIG. 2: Receiver-operating characteristic curve for Model 1a.

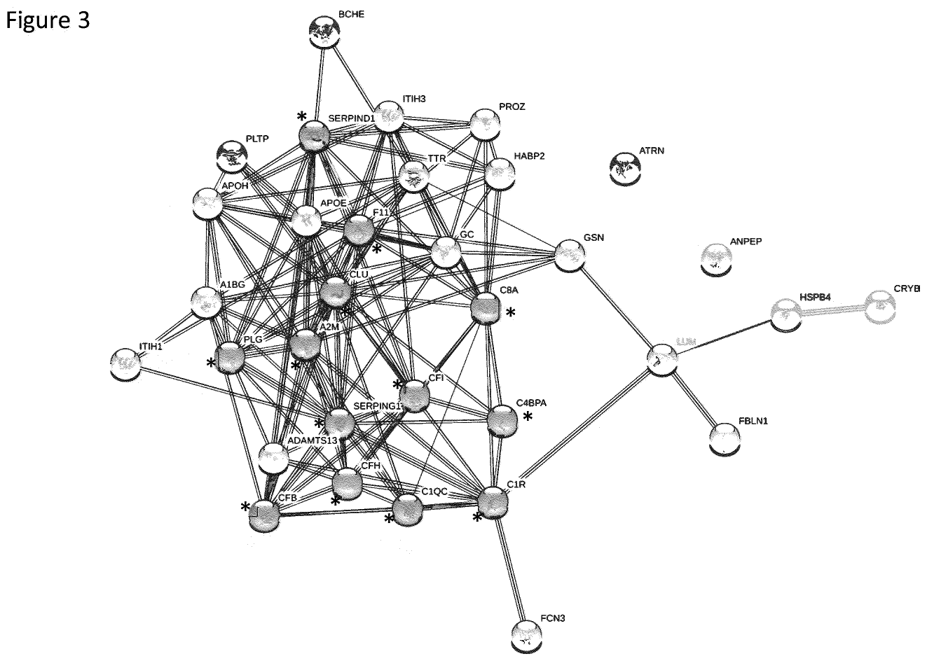

FIG. 3: STRING network analysis of protein-protein interactions for differentially expressed proteins (following FDR adjustment) between CHR-T and CHR-NT in EU-GEI.

The network nodes are proteins (proteins implicated in the complement and coagulation cascades are highlighted in red (marked with *)). The edges represent functional associations between proteins, and the colour of each edge represents the source of evidence for that association, including fusion evidence; co-occurrence evidence; experimental evidence; and text mining evidence.

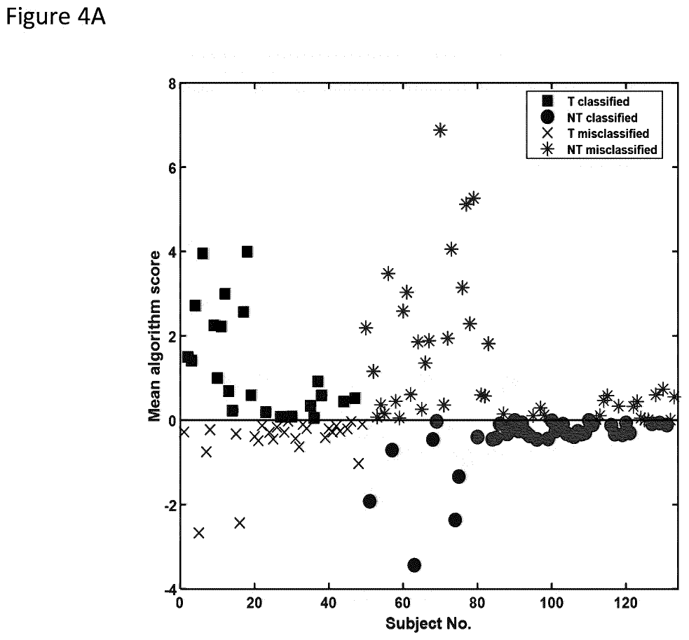

FIG. 4: Mean algorithm scores (A) and receiver-operating characteristic curve (B) for Model 1b. T: ultra high-risk participants who transitioned to first episode psychosis; NT: ultra high-risk participants who did not transition.

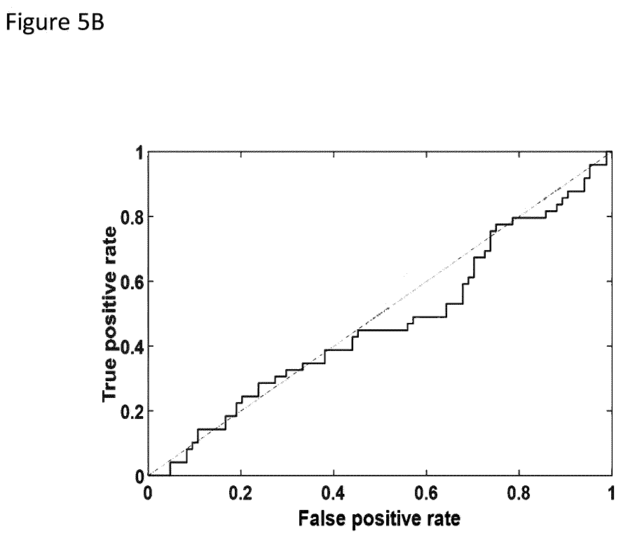

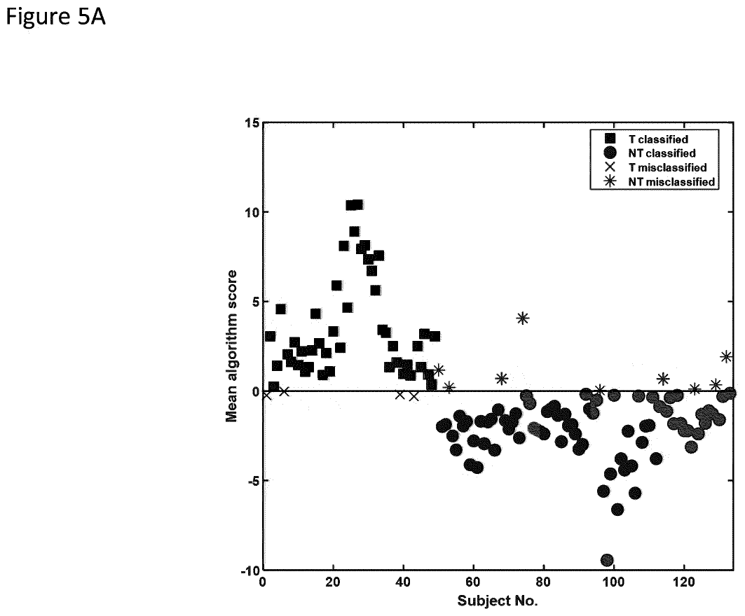

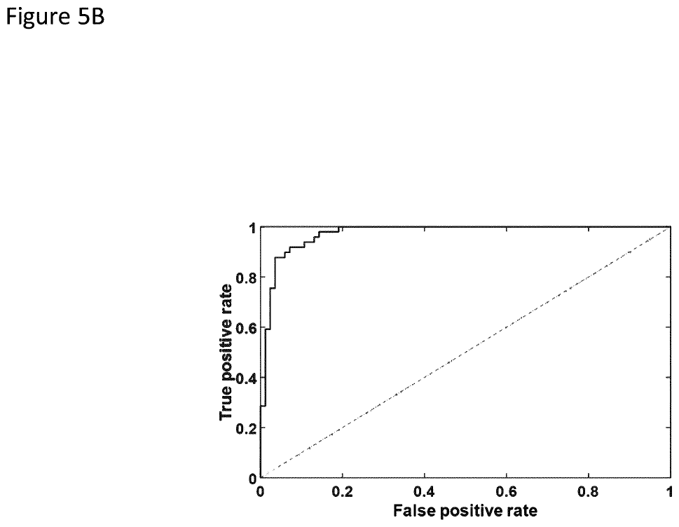

FIG. 5: Mean algorithm scores (A) and receiver-operating characteristic curve (B) for Model 1c.: ultra high-risk participants who transitioned to first episode psychosis; NT: ultra high-risk participants who did not transition.

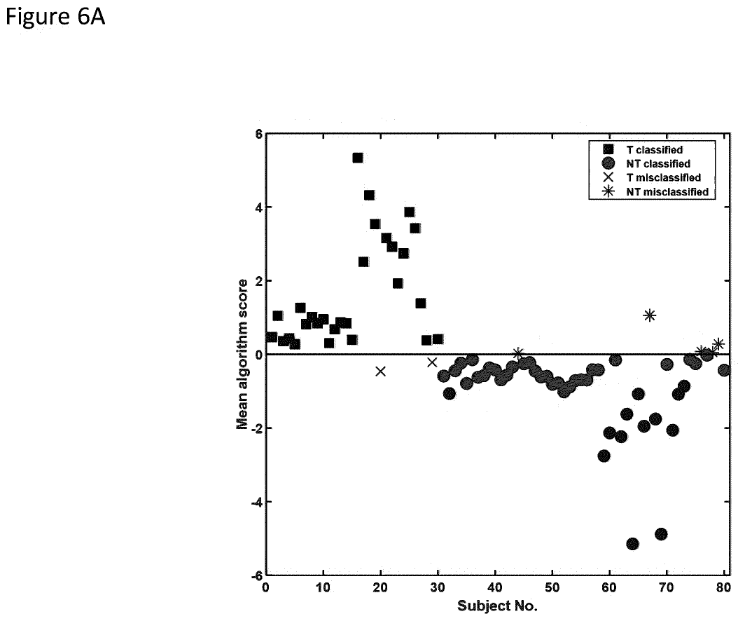

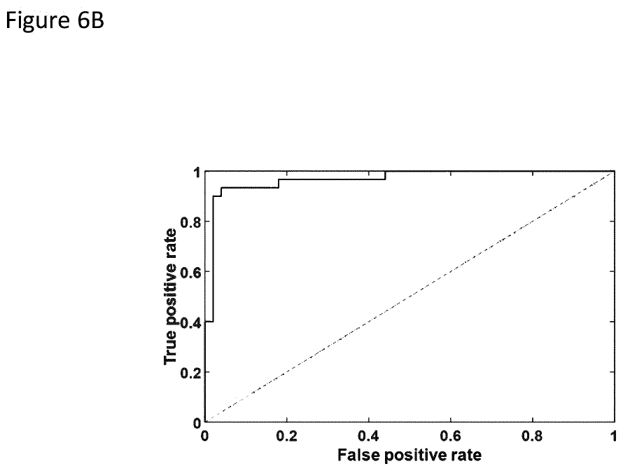

FIG. 6: Mean algorithm scores (A) and receiver-operating characteristic curve (B) for Model 1d in training dataset (all sites except London): ultra high-risk participants who transitioned to first episode psychosis; NT: ultra high-risk participants who did not transition.

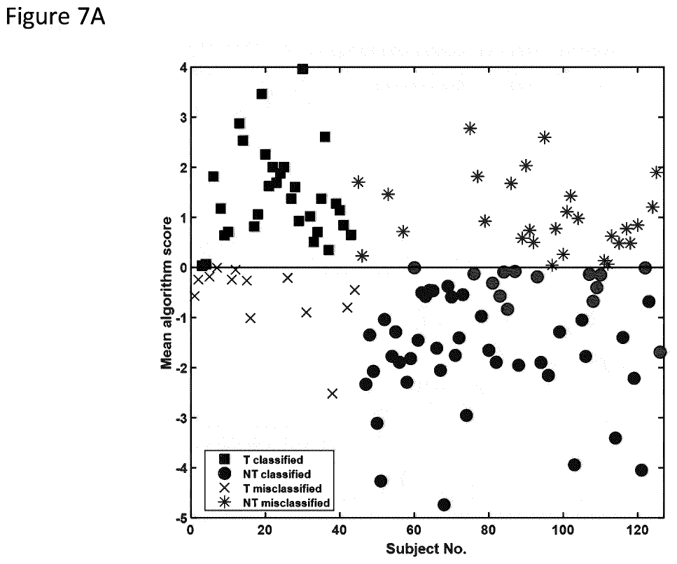

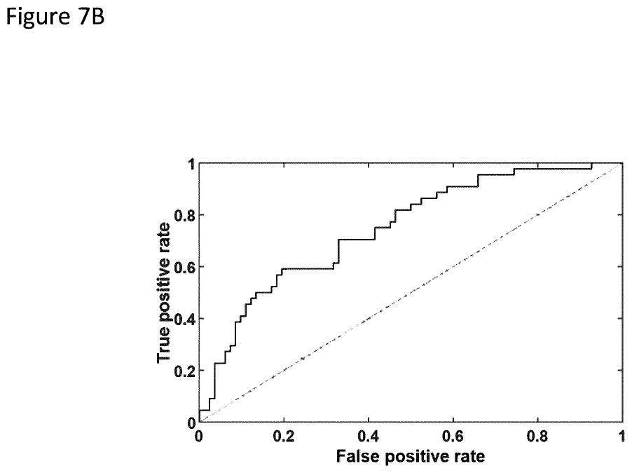

FIG. 7: Mean algorithm scores (A) and receiver-operating characteristic curve (B) for Model 2. T: ultra high-risk participants who transitioned to first episode psychosis; NT: ultra high-risk participants who did not transition.

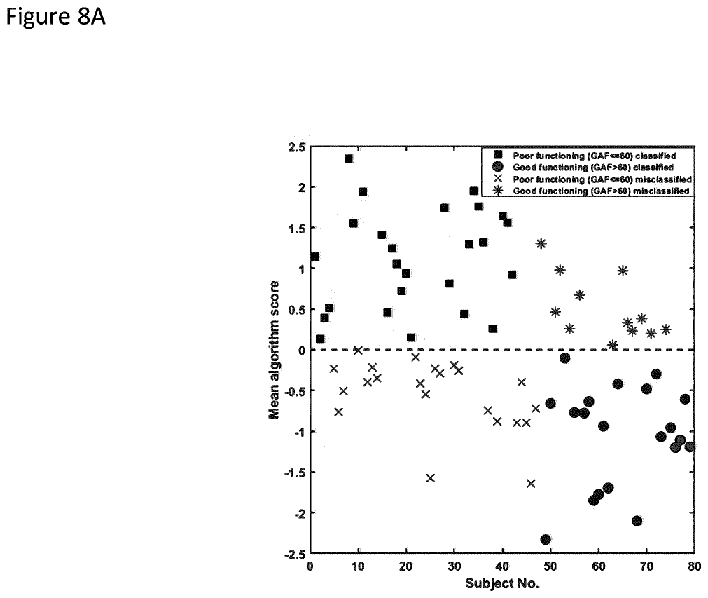

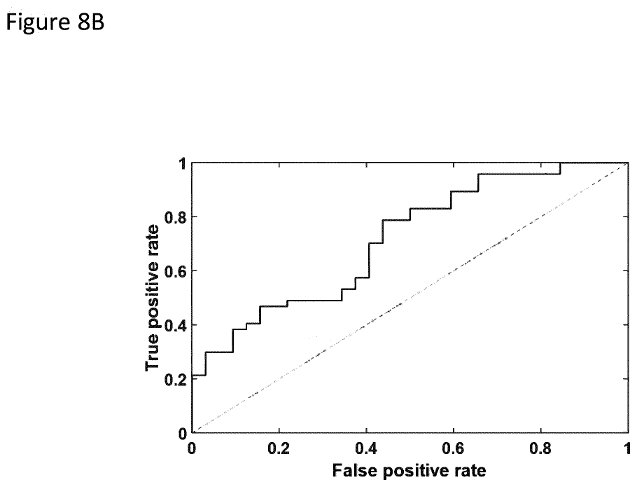

FIG. 8: Mean algorithm scores (A) and receiver-operating characteristic curve (B) for Model 3. Poor: General Assessment of Functioning disability score ≤60 at 2 years; Good: General Assessment of Functioning disability score >60 at 2 years.

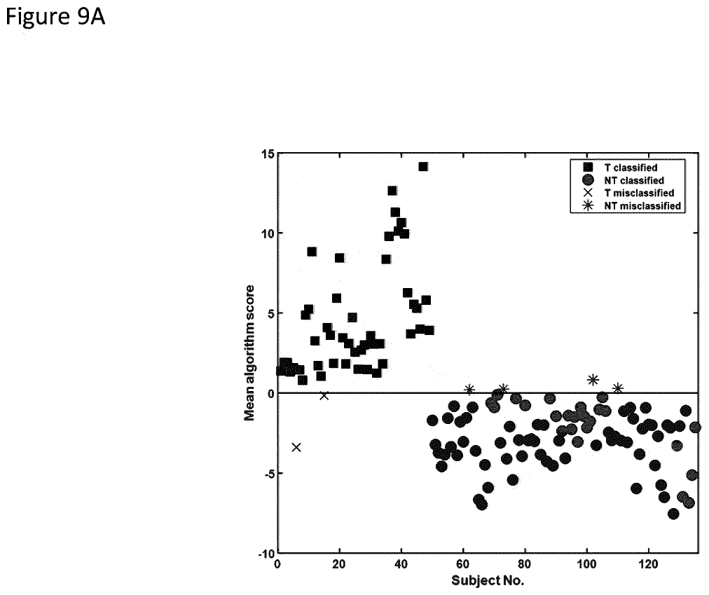

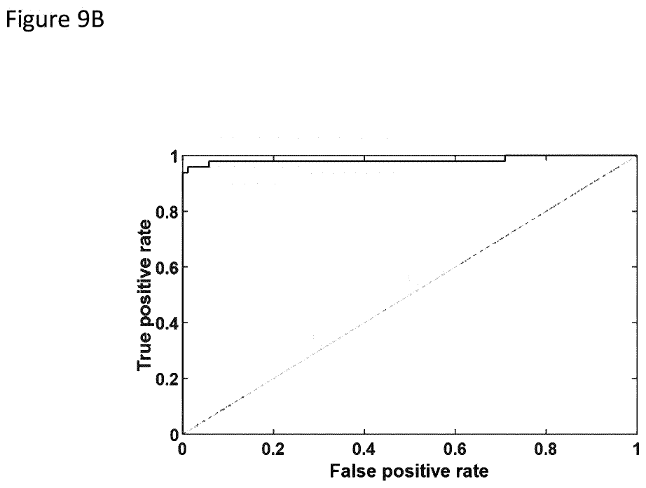

FIG. 9: Mean algorithm scores (A) and receiver-operating characteristic curve (B) for Model 4. T: ultra high-risk participants who transitioned to first episode psychosis; NT: ultra high-risk participants who did not transition.

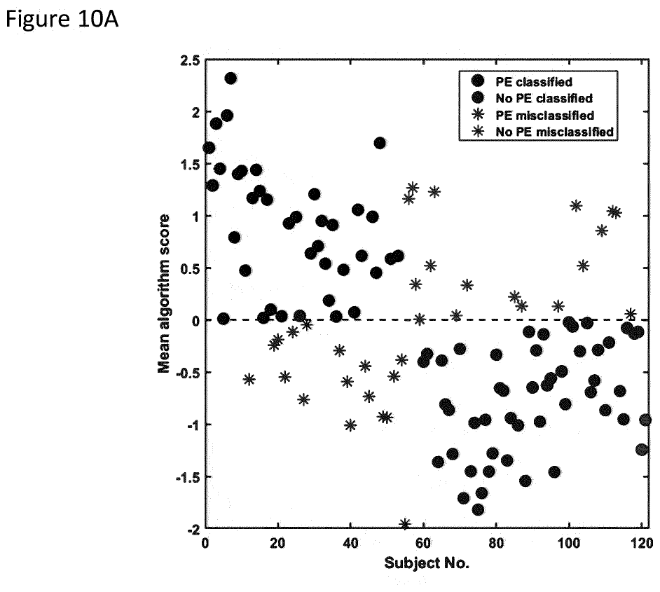

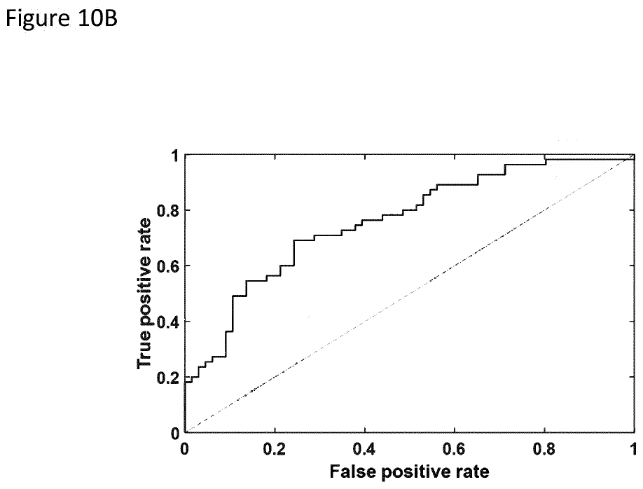

FIG. 10: Mean algorithm scores (A) and receiver-operating characteristic curve (B) for Model 5. PE: Definite psychotic experiences at 18; No PE: No psychotic experiences at 18.

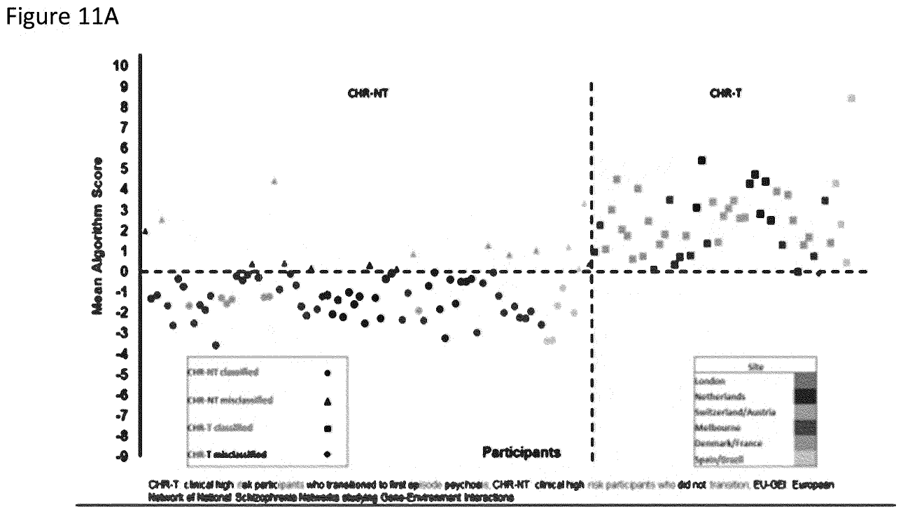

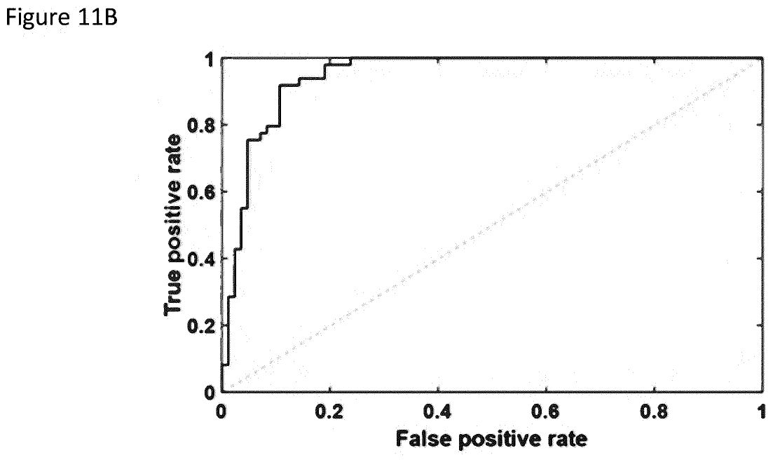

FIG. 11a: mean algorithm scores for Model 11a. FIG. 11b: Receiver-operating characteristic curve for Model 1a.

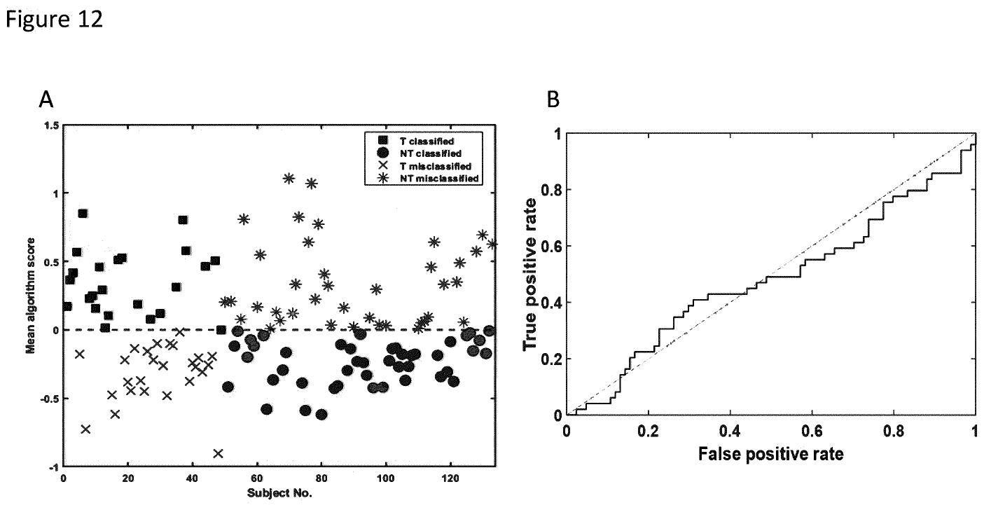

FIG. 12: Mean algorithm scores and class predictions (A) and receiver-operating characteristic curve (B) for Model 1b: clinical data. T: clinical high-risk participants who transitioned to first episode psychosis; NT: clinical high-risk participants who did not transition.

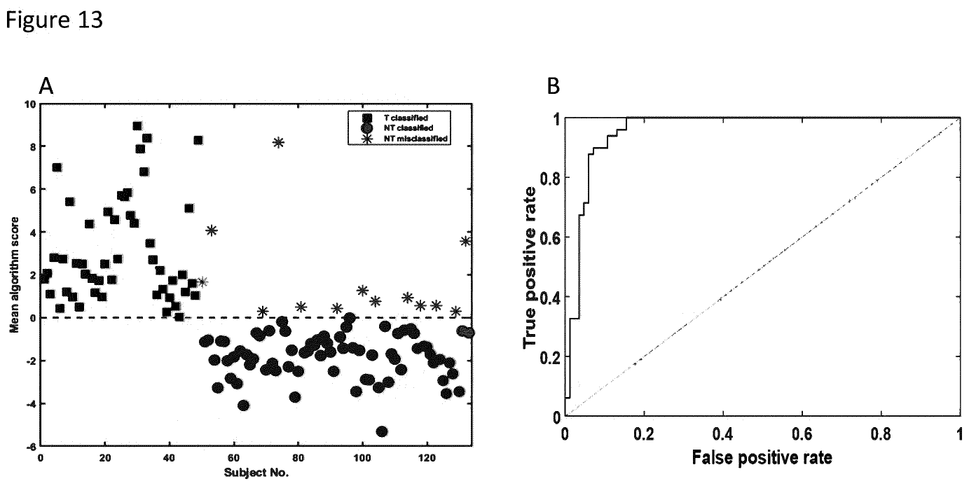

FIG. 13: Mean algorithm scores and class predictions (A) and receiver-operating characteristic curve (B) for Model 1c: proteomic data. T: clinical high-risk participants who transitioned to first episode psychosis; NT: clinical high-risk participants who did not transition.

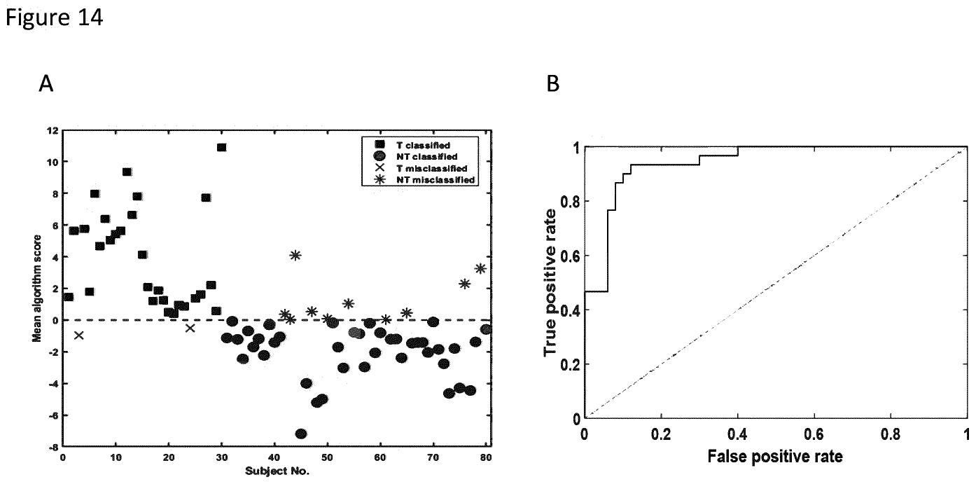

FIG. 14: Mean algorithm scores and class predictions (A) and receiver-operating characteristic curve (B) for Model 2a: proteomic (non-London). T: clinical high-risk participants who transitioned to first episode psychosis; NT: clinical high-risk participants who did not transition.

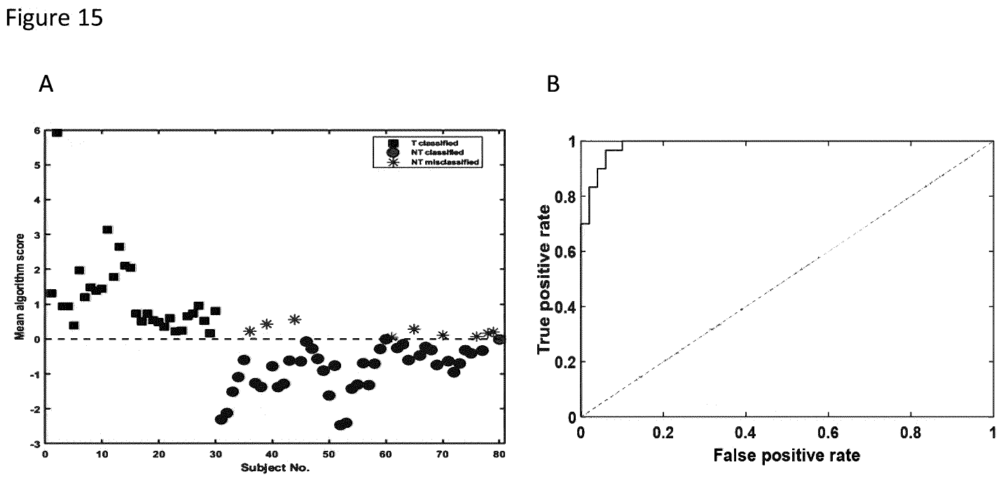

FIG. 15: Mean algorithm scores and class predictions (A) and receiver-operating characteristic curve (B) for Model 2b: parsimonious (10-predictor) proteomic model, training data (non-London).

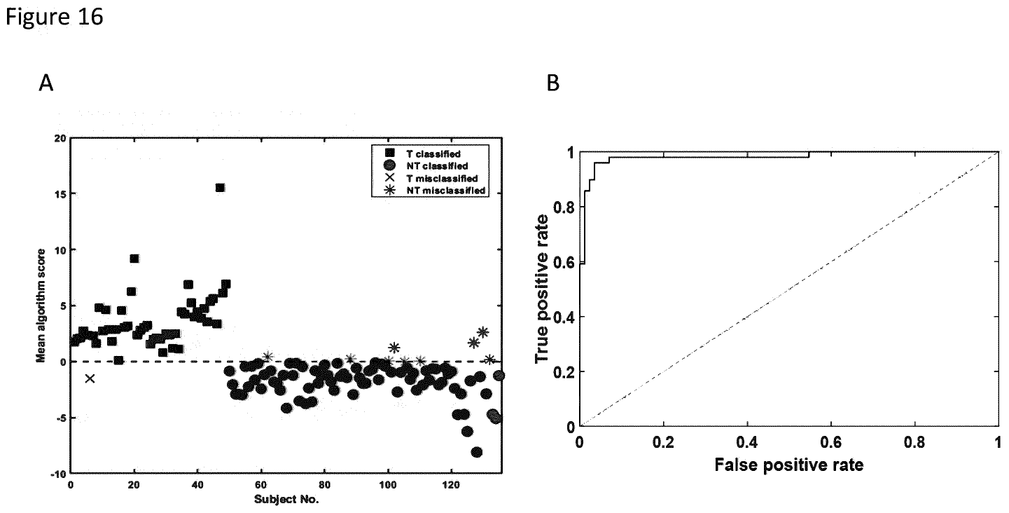

FIG. 16: Mean algorithm scores and class predictions (A) and receiver-operating characteristic curve (B) for Model 3: replication. T: clinical high-risk participants who transitioned to first episode psychosis; NT: clinical high-risk participants who did not transition.

### EXAMPLE 1—DEVELOPMENT OF PROTEOMIC PREDICTION MODELS FOR OUTCOMES IN THE CLINICAL HIGH RISK STATE AND PSYCHOTIC EXPERIENCES IN ADOLESCENCE: MACHINE LEARNING ANALYSES IN TWO NESTED CASE-CONTROL STUDIES

### Summary

Background: Biomarkers for prediction of outcomes in people at risk of psychosis would inform the clinical management of this group.
Methods: We conducted two nested case-control studies. The first study was nested within the European Network of National Schizophrenia Networks Studying Gene-Environment Interactions (EU-GEI) and comprised 133 clinical high-risk (CHR) participants, of whom 49 transitioned to psychosis. The second study was nested within the Avon Longitudinal Study of Parents and Children (ALSPAC) and comprised 121 participants who did not report psychotic experiences (PEs) at age 12, of whom 55 later reported PEs at age 18. Baseline plasma samples in EU-GEI and age 12 plasma samples in ALSPAC were analysed using mass spectrometry. Support vector machine algorithms were used to develop models for prediction of transition and functional outcome in EU-GEI, and PEs at age 18 in ALSPAC.

Outcomes: In the CHR sample, using 65 clinical and 166 proteomic features a model demonstrated excellent performance for prediction of transition status (area under the receiver-operating curve [AUC] 0.96, positive predictive value [PPV] 81.8%, negative predictive value [NPV] 94.9%). A model based on the ten most predictive proteins accurately predicted transition status in training (AUC 0.97, PPV 84.8%, NPV 95.7%) and withheld data (AUC 0.93, PPV 80.0%, NPV 90.9%). A model using the same 65 clinical and 166 proteomic features predicted functional outcome with AUC 0.72 (PPV 67.6%, NPV 47.6%). In the general population sample, a model using 265 proteomic features predicted psychotic experiences at age 18 with AUC 0.76 (PPV 69.1%, NPV 74.2%).

Interpretation: Proteomic markers may contribute to prediction of outcomes in individuals at risk of psychosis.

Funding: Health Research Board Clinician Scientist Award to DRC; European Community's Seventh Framework Programme (EU-GEI); UK Medical Research Council, Wellcome Trust (ALSPAC).

### Introduction

Early detection of people with psychotic disorders may improve their clinical outcomes.¹ There has been a focus on the clinical high-risk (CHR) state² with the aim of identifying vulnerable individuals and offering clinical interventions.^{3, 4} 16-35% of CHR individuals develop first-episode psychosis (FEP) within 3 years^{5, 6} and the CHR state is often associated with co-morbid depressive and anxiety disorders^{7, 8} as well as functional impairment.^{9, 10} Studies have also characterised an ‘extended psychosis phenotype’ which includes individuals with psychotic experiences (PEs),¹¹ psychotic symptoms that may occur in the general population or at less severe points on the psychosis continuum, with or without help-seeking. PEs are associated with increased risk of psychotic and non-psychotic disorders,¹² suicidal behaviour¹³ and reduced functioning.^{14, 15}

Biomarkers may aid prediction of outcomes for at-risk individuals.¹⁶ Blood-based studies associate inflammatory and immune-related processes with development of psychosis.¹⁷⁻¹⁹ This is supported by proteomic studies in schizophrenia implicating the acute phase response, glucocorticoid receptor signalling, coagulation and lipid metabolism.^{20, 21} Proteomic studies in those who develop PEs provide evidence for early dysregulation of the complement system,^{22, 23} which has been implicated in schizophrenia^{24, 25} and other mental disorders.²⁶

We aimed to apply proteomic methods to assess differential protein expression in CHR individuals who do and do not develop psychosis, and to develop predictive models for transition and functional outcome. We employed similar methods for the broader phenotype of PEs in a general population sample. For predictive modelling, we used machine learning techniques such as have been used for prediction of functional outcome in CHR²⁷ and increasingly in psychiatry.^{28,29}

### Methods

### Study 1: CHR Sample

### Participants and Study Design

The European network of national schizophrenia networks studying Gene-Environment Interactions (EU-GEI) is a collaborative project studying gene-environment interactions in schizophrenia. Work Package 5 comprises a prospective study of CHR individuals followed for up to 6 years, across 11 sites in Europe, Australia and Brazil.^{30,31, 32} Participants with CHR symptoms were referred by their local mental health service and were eligible to participate if they met CHR criteria as defined by the Comprehensive Assessment of At-Risk Mental States³³ (CAARMS). Exclusion criteria were: current or past psychotic disorder; symptoms explained by a medical disorder or drug or alcohol use; IQ<60. Plasma samples were obtained at baseline and participants followed clinically for up to six years, with clinical assessments performed at baseline, 12 and 24 months. Accrual began in September 2010 and the last baseline assessment was performed in July 2015. The present study was a nested case-control study of participants who provided plasma samples at baseline, comparing samples from CHR participants who transitioned to psychosis on follow-up (CHR-T, n=49) with a control group of randomly-selected participants who did not (CHR-NT, n=84).

### Outcomes

Transition status: Transition was defined as the onset of non-organic psychotic disorder as determined by the CAARMS. Assessors were not systematically blinded to transition status since, in some cases, clinical services had contacted the research team to advise that transition had occurred. For participants who developed psychosis after 24 months, transition status was determined by contacting the clinical team or from clinical records. Functional outcome: We used the General Assessment of Functioning (GAF)^{34, 35} disability subscale, recorded at follow-up assessment closest to two years from baseline. For use as a classification target variable (and in line with previous approaches³⁶) the score was dichotomised into ‘poor functioning’ (≤60 points; i.e. moderate or severe impairment) or ‘good functioning’ (>60 points; i.e. mild or no impairment).

### Clinical Measures

Baseline clinical data included sociodemographic data, the GAF subscales for symptoms and disability,^{34, 35} the Scale for Assessment of Negative Symptoms (SANS),³⁷ the Brief Psychiatric Rating Scale (BPRS)³⁸ and the Montgomery-Asberg Depression Rating Scale (MADRS).³⁹

### Sample Preparation

Protein digestion and peptide purification was performed as previously described⁴⁰. Laboratory staff were blind to case/control status.

### Proteomic Analysis

We used discovery-based proteomic methods, namely data-dependent acquisition (DDA), as described previously²². Briefly, 5 μl from each sample was injected on a Thermo Scientific Q-Exactive mass spectrometer, connected to a Dionex Ultimate 3000 (RSLCnano) chromatography system, and operated in DDA mode for label-free liquid chromatography mass spectrometry.^{22, 23, 40-42}

### Enzyme-Linked Immunosorbent Assay (ELISA) Validation

We assessed nine proteins in plasma samples from the same CHR-T and CHR-NT participants using ELISA.

### Replication

In a partial replication of the mass spectrometry experiment, we analysed samples from mostly the same group of CHR-T participants (2 of the 49 cases were different, otherwise the CHR-T participants were the same) and an entirely different group of CHR-NT participants (n=86).

### Study 2: General Population Sample

### Participants and Study Design

The Avon Longitudinal Study of Parents and Children (ALSPAC) is a prospective birth cohort study.^{43, 44} Pregnant women resident in Avon, UK with expected dates of delivery between 1 Apr. 1991 to 31 Dec. 1992 were invited to participate with a total sample of over 15,000 pregnancies. The study website contains details of available data (http://www.bristol.ac.uk/alspac/researchers/our-data/). We previously analysed age 12 plasma samples from young people who did and did not report PEs at age 18,^{22, 23} finding several differentially expressed proteins in a data-independent acquisition (DIA) analysis focused on proteins of the complement pathway. In the present study we performed DDA analyses rather than DIA to achieve broader proteome coverage.

### Outcome

Psychotic experiences: PEs were assessed at 12 and 18 years using the semi-structured Psychosis-Like Symptom Interview¹¹ and rated as not present, suspected or definitely psychotic. Cases were participants who did not report PEs at age 12 but reported at least one definite PE at age 18. Controls were randomly selected age-matched participants who did not report PEs at age 12 nor 18.

### Sample Preparation

Age 12 plasma samples were prepared for mass spectrometry as previously described.²²

### Bioinformatics and Statistical Analysis

### Demographic and Clinical Data

Baseline demographic and clinical data were tested for differences using the 2-sided t-test for continuous and χ² test for categorical variables in SPSS v.25 (Armonk, N.Y., USA) with α=0.05. In Study 1, baseline clinical data comprised 65 variables including the GAF subscales for symptoms and disability, and total and individual item scores for the SANS, BPRS and MADRS. In Study 2, data were obtained for sex, ethnicity, maternal social class and body mass index (BMI) at age 12. Missing clinical data were replaced using the mean (for continuous) or mode (for categorical variables).

### Proteomic Data

Label free quantification (LFQ) was performed in Max Quant (v.1.5.2.8)^{45, 46} as described.⁴⁰ Proteins that were identified with at least two peptides (one uniquely assignable to the protein) and quantified in >80% of samples were taken forward for quantification. LFQ values were log 2-transformed and missing values imputed using the imputeLCMD package v.2.0⁴⁷ in RStudio (Boston, Mass., USA; http://www.rstudio.com/). Values were converted to z-scores and winsorised within ±3z.

### Differential Expression

To determine differential expression, analysis of covariance (ANCOVA) was performed in Stata 15 (College Station, Tex., US) comparing mean LFQ value in cases and controls for each identified protein. In Study 1, covariates were age, sex, BMI and years in education. In Study 2, covariates were sex, maternal social class and age 12 BMI. P-values were corrected for multiple comparisons using the Benjamini-Hochberg procedure⁴⁸ with false discovery rate (FDR) of 5%.

### Support Vector Machine (SVM) Models

We used the open-source machine learning software Neurominer v.1.0 (https://www.pronia.eu/neurominer/) for MatLab 2018a (MathWorks Inc, USA) to generate predictive models, with area under the receiver-operating curve (AUC) as the performance criterion for evaluation and optimisation. Continuous variables were converted to z-scores for feature scaling and winsorised within ±3z. Random-label permutation analysis⁴⁹⁻⁵¹ with 250 permutations was used to verify predictive models against null models and derive p-values for model significance.

### SVM Model 1

We used an L2-regularised SVM algorithm to develop a classification model predicting transition outcome. We incorporated geographical generalisability using repeated nested cross-validation with a leave-site-out approach in the outer loop as previously described.⁵⁰ We used the LIBLINEAR program with L2 regularisation to attenuate risk of over-fitting⁵² whereby weightings of non-predictive features are minimised, but not reduced to zero (thus more closely modelling the biological effects of functionally inter-related proteins). Given the unbalanced group sizes, the hyperplane was weighted (increasing the misclassification penalty in the minority class) which reduces the risk of bias.′ A priori covariates were age, sex, BMI and years in education.

### SVM Model 2

Concentrations derived from ELISA were used as features in an L2-regularised SVM algorithm with cross-validation and covariates as for Model 1.

### SVM Model 3

We used an L2-regularised SVM algorithm to derive a classification model predicting functional outcome at 2 years: poor (GAF≤60) vs. good (GAF>60) functioning. Features and covariates were as for Model 1. Compared to transition status, fewer participants had data available for functional outcome (n=79). Therefore, this model used five-fold repeated nested cross-validation with five inner and five outer folds, irrespective of study site.

### SVM Model 4

We developed an L2-regularised SVM model for prediction of transition status using the clinical and proteomic features in the replication dataset. This model used leave-site-out repeated nested cross-validation as for Model 1. In addition to age, sex, BMI and years in education, we also adjusted for ethnicity and tobacco use due to evidence of baseline differences for these characteristics.

### SVM Model 5

We developed an L2-regularised SVM model predicting PEs at age 18 in ALSPAC using DDA proteomic data at age 12. Repeated nested cross-validation with five inner and five outer folds was used to derive the model. Sex, maternal social class and age 12 BMI were covariates.

### Results

### Study 1: EU-GEI (CHR Sample)

### Sample Characteristics

The EU-GEI cohort included 344 CHR participants, of whom 65 (18.9%) developed psychosis on follow-up. 57 transitioned within two years as defined by the CAARMS. In the eight who transitioned after two years, transition status was determined by contact with the clinical team or from clinical records.

Our subsample comprised 49 CHR-T and 84 CHR-NT participants. Characteristics of included and non-included participants are compared in Table 5. At baseline, included participants had higher mean total SANS composite and global scores and total BPRS score compared to non-included participants, but were otherwise comparable.

Among included participants, there was evidence that the mean baseline total BPRS score was higher in included CHR-T compared to CHR-NT. There was no evidence of differences between the groups on other symptom measures, socio-demographic features, cannabis use or medication use (Table 1). The median duration from baseline to transition was 219 days (interquartile range 424 days).

### Differential Expression

Of 345 proteins identified, 166 were quantified in >80% of samples. There was nominally significant (p<0.05) differential expression for 56 proteins in CHR-T vs. CHR-NT, of which 35 remained significant after 5% FDR adjustment (Table 6. Proteins with FDR-adjusted p<0.001 (and associated mean fold change in CHR-T vs. CHR-NT) included: alpha-2-macroglobulin (Alpha-2-macroglobulin, 0.33), immunoglobulin heavy constant mu (Immunoglobulin heavy constant mu, 0.41), complement C8 alpha chain (Complement component 8 alpha chain, 1.48), vitamin D binding protein (VTDB, 1.43), complement C1q subcomponent subunit C (Complement C1q subcomponent subunit C, 1.53), plasminogen (1.29), clusterin (1.29), fibulin-1 (1.52), phospholipid transfer protein (Phospholipid transfer protein, 0.67), complement C1r subcomponent (1.27). FIG. 3 shows results of a STRING database⁵⁴ protein interaction network analysis for differentially expressed proteins. The topmost implicated pathway was the complement and coagulation cascade (Table 7).

### SVM Model 1

An SVM classification model predicted transition status based on 65 clinical and 166 proteomic features (Model 1a) with AUC 0.96 (p<0.004). Further performance metrics are presented in Table 2. FIG. 1 shows class prediction stratified by site and FIG. 2 the receiver-operating characteristic curve. Table 3 lists the 10% highest-weighted features based on mean feature weighting across all models selected in the inner loop.

We examined the predictive value of the clinical and the proteomic data separately by generating models based on each dataset individually. The clinical model (Model 1b) poorly predicted transition outcome (AUC 0.47, p=0.7; Table 2 and FIG. 4). The proteomic model (Model 1c) demonstrated excellent predictive performance (AUC 0.97, p<0.004; Table 2 and FIG. 5).

We next sought to develop a more parsimonious model based on a subset of 10 predictive features, and to test such a model in unseen data. As the largest site, we chose London to be the test site. To derive the ten highest-weighted proteins, an L2-regularised SVM model was trained using the proteomic data from all sites except London (CHR-T n=30, CHR-NT n=50), with leave-site-out cross-validation and adjustment for the same covariates as for Model 1a. The resulting AUC was 0.94, p<0.004 (sensitivity 86.7%, specificity 94.0%, balanced accuracy 90.3%, PPV 89.7%, NPV 92.2%, positive likelihood ratio 14.4, negative likelihood ratio 0.1). The ten highest-weighted features were: Alpha-2-macroglobulin, Immunoglobulin heavy constant mu, C4b-binding protein alpha chain, Phospholipid transfer protein, transthyretin, VTDB, vitamin K dependent protein S (PROS), Beta-crystallin B2, coagulation factor XII and clusterin. A reduced model using these ten features (Model 1d) was trained on data from all sites except London with AUC 0.97, p<0.004 (Table 2 and FIG. 6). This model predicted transition status in the withheld London sample (CHR-T n=19, CHR-NT n=34) with AUC 0.93 (Table 2).

### ELISA Validation

Three of the nine proteins assessed by ELISA showed statistically significant mean differences between CHR-T and CHR-NT (Alpha-2-macroglobulin p=0.0016, C1r p=0.0084, plasminogen p=0.0196; Table 8).

### SVM Model 2

Complete ELISA data were available for 126 participants (CHR-T n=44, CHR-NT n=82). This model predicted transition status with AUC 0.76, p<0.004 (Table 2; FIG. 7).

### SVM Model 3

In the 79 participants with outcome data available, this model predicted 2-year functional outcome with AUC 0.72, p=0.008 (Table 2; FIG. 8). The 10% highest-weighted features are listed in Table 3.

### SVM Model 4

Sample characteristics of the replication dataset are described in Tables 9 and 10. Of 485 proteins identified, 119 were quantified in >80% of samples. In ANCOVA adjusted for age, sex, BMI, years in education, tobacco use and ethnicity, 82 proteins showed nominally significant differential expression (p<0.05) in CHR-T vs. CHR-NT of which 78 remained significant following 5% FDR adjustment (Table 11).

Due to differences in protein identifications, it was not possible to apply Models 1a-d in the replication dataset. We generated a new L2-regularised SVM model (adjusted for age, sex, BMI, years in education, tobacco use and ethnicity) using these 119 proteomic features and the same 65 clinical features as for Model 1a. This model demonstrated excellent performance for prediction of transition status (AUC 0.98, p<0.004; Table 2 and FIG. 9). The highest-weighted 10% of features are listed in Table 12. Proteins among the highest-weighted 10% of features in both Model 1a and Model 4, and weighted in similar directions, included Alpha-2-macroglobulin, Immunoglobulin heavy constant mu, clusterin, C4b-binding protein alpha chain and complement component 6 (Complement component 6).

### Further Models

A model based only on the top 5 proteins when adjusting for age, sex, BMI and years in education (comprising A2M, IGHM, C4BPA, PLTP, transthyretin) was trained on the non-London samples (n=80) and predicted transition status with AUC 0.95 and balanced accuracy 94.3% (PPV 87.9%, NPV 97.9%). When tested on the withheld London samples (n=53) this model predicted transition status with AUC 0.92 and balanced accuracy 91.8% (PPV 89.5%, NPV 94.1%).

Another model based only on the top 5 proteins unadjusted for covariates (comprising A2M, IGHM, C4BPA, vitamin K dependent protein S, fibulin-1) was trained on the non-London samples (n=80) and predicted transition status with AUC 0.97 and balanced accuracy 86.3% (PPV 70.7%, NPV 97.4%). When tested on the withheld London samples (n=53) this model predicted transition status with balanced accuracy 90.1% (PPV 84.2%, NPV 93.9%).

A model based only on the top 2 proteins when adjusting for age, sex, BMI and years in education (comprising A2M and IGHM) was trained on the non-London samples (n=80) and predicted transition status with AUC 0.94 and balanced accuracy 86.0% (PPV 75.0%, NPV 93.2%). When tested on the withheld London samples (n=53) this model predicted transition status with AUC 0.91 and balanced accuracy 87.4% (PPV 77.3%, NPV 93.5%).

### Study 2: ALSPAC (General Population Sample)

### Sample Characteristics

The total sample comprised 65 cases and 67 controls. Eleven samples were excluded due to poor protein identification profiles, resulting in 55 cases and 66 controls. There was evidence that cases were more likely to be female, but no evidence of differences in ethnicity, maternal social class or age 12 BMI (Table 13).

### Differential Expression

Of 506 proteins identified, 265 were quantified in >80% of samples. There was nominally significant (p<0.05) differential expression of 40 proteins at age 12 (Table 14) of which five remained significant after 5% FDR adjustment (mean fold change in cases vs. controls): C4b-binding protein alpha chain (0.77), serum paraoxonase/arylesterase 1 (0.80), Immunoglobulin heavy constant mu (0.78), inhibin beta chain (1.31) and clusterin (0.92).

### SVM Model 5

An SVM model based on proteomic features from age 12 plasma samples predicted PE status at age 18 with AUC 0.76, p<0.004 (Table 2 and FIG. 10). The 10% highest-weighted features are listed in Table 3.

### Discussion

We report evidence of differential expression of multiple proteins at baseline between CHR individuals who developed psychosis compared to those who did not, with particular implication of the complement and coagulation cascade. We used machine learning algorithms incorporating clinical and proteomic data to predict transition (AUC 0.96). Proteomic features were of greater predictive value than the included clinical features. A reduced model using data for 10 highly predictive proteins showed excellent predictive performance in training (AUC 0.97) and testing on withheld data (AUC 0.93). We also developed models for prediction of functional outcome in the same CHR population (AUC 0.72) and for prediction of PEs in a longitudinal birth cohort (AUC 0.76). Our results have clinical and aetiopathogenic implications.

Although a minority of CHR individuals transition to FEP,^{5, 6} the CHR state is a strong risk indicator for psychosis.′ Accurate identification of those at greatest risk of transition would facilitate targeting preventative interventions. Models based on clinical data have previously shown some value for prediction of transition and functional outcome in CHR.⁵⁶⁻⁶⁰ A previous study combined CHR criteria with data on cognitive disturbances, achieving AUC of 0.81 for prediction of transition.⁶¹ Accuracy has been further augmented using neuroimaging^{36, 62-64} and neurocognitive⁶⁵ data. However, blood-based tests have the advantage of greater accessibility. A previous investigation found a panel of 15 proteins using immunoassays that distinguished between CHR individuals who did and did not transition with AUC 0.88.⁶⁶ A further study used blood-based biomarkers to predict onset of schizophrenia with AUC 0.82, which improved to 0.90 when the CAARMS positive symptoms subscale was included in the model.¹⁹

We developed a parsimonious model using data from 10 highly predictive proteins which accurately predicted transition outcome. With further validation, these markers may contribute to individualised prognostic scores and stratification strategies to improve risk estimation.′ Notably, the models for transition that incorporated proteomic data had high sensitivity. In implementation, this would require balancing against the costs of unnecessary treatment of false positive individuals. However, interventions in CHR generally have a psychosocial focus⁶⁸⁻⁷¹ and may have utility even in those who will not ultimately transition.

Regarding pathogenesis, our study provides the first mass spectrometry-derived evidence of differential protein expression associated with transition in CHR. We find particularly strong indication for the complement and coagulation cascades, which have previously been implicated in schizophrenia^{20, 24, 72-77} and preceding PEs.^{22, 23} The primary causes of these changes remain unknown, but are consistent with evidence for raised inflammatory tone preceding psychosis and other mental disorders^{19, 66, 78-84} and the vulnerability associated with genetic variation of complement C4 in schizophrenia.′ In our study, several complement pathway proteins emerged as important predictors of transition including C4b-binding protein alpha chain, Complement C1q subcomponent subunit C, C1r of the antibody-antigen complex mediated pathway, key regulatory protease CFI, ficolin-3 and terminal pathway components Complement component 6 and Complement component 8 alpha chain. These proteins arise from common proteolytic pathways or interact with coagulation proteins plasminogen (positively associated with transition) and vitamin K-dependent protein S (negatively associated with transition), supporting hypotheses of activation of coagulation in psychosis.⁷⁵

The strongest predictor of transition was Alpha-2-macroglobulin (reduced in CHR-T), a protease inhibitor with diverse functions including inhibition of pro-inflammatory cytokines such as IL1β,⁸⁵ which has been shown to be raised in FEP.⁸⁶ Alpha-2-macroglobulin is also a key coagulation inhibitor^{87, 88} and thus links functionally to our observations of elevated plasminogen in CHR-T. This is intriguing in light of evidence that blood-derived plasminogen drives brain inflammation⁸⁹ and complement activation.⁹⁰ In models of multiple sclerosis, blood-brain barrier disruption facilitates transfer of fibrinogen into the brain where it is deposited as fibrin, causing local inflammation.⁹¹ Our findings suggest a procoagulant phenotype in CHR-T and, given evidence for blood-brain barrier disruption in psychosis,⁹² the effects of fibrin provide possible aetiopathogenic mechanisms and novel therapeutic avenues,⁹³ but this will require further confirmation.

We validated differential expression of Alpha-2-macroglobulin, C1r and plasminogen using ELISA. The ELISA-based SVM model demonstrated acceptable, though reduced, predictive accuracy. This may reflect the reduced sensitivity of ELISA and the inability to accurately quantify specific protein isoforms. It is intriguing that several proteins in the highest-weighted 10% of features in the original dataset were similarly highly weighted (and in similar directions) for prediction of PEs in the general population (C4b-binding protein alpha chain, vitamin K-dependent protein S, Alpha-2-macroglobulin and Immunoglobulin heavy constant mu). This could tentatively suggest a degree of similarity in certain proteomic changes between non-clinical youth who develop PEs and help-seeking CHR individuals who develop psychosis, but will require further investigation. More widely, our results are in keeping with studies in bipolar disorder and depression reporting reductions in Alpha-2-macroglobulin, IgM and C4b-binding protein alpha chain.′ Thus these changes may be in keeping with a general vulnerability to psychiatric disorder, outside of the psychosis spectrum.

Our study is not without its limitations. Firstly, the poor performance of the clinical model likely relates to the included features. It is probable that other clinical data (for example, individual CAARMS items) would lead to improved predictive ability. However, our primary aim was to investigate the role of proteomic predictors. Secondly, we could not access a similar sample in which we could test the external predictive ability of the models. Thirdly, our replication experiment was partial, since we were unable to access an entirely new set of CHR-T cases. Finally, it is possible that childhood adversity at least partially mediates the changes we observe,^{23, 95-97} but this will require further study.

In conclusion, we have developed models incorporating proteomic data to contribute to prediction of transition and functional outcome in CHR. In a longitudinal birth cohort, several of the same proteins also contributed to prediction of PEs. Further studies are required to validate these findings, evaluate their causes and elucidate amenable targets for prediction and prevention of psychosis.

### Evidence in Context

Evidence Before this Study

Schmidt et al (2017) conducted a systematic review of studies (published until October 2015) that developed predictive models for onset of psychosis in people at clinical high risk (CHR) using clinical, biological, cognitive and environmental predictors or combinations thereof. To identify further studies published since this review, we performed a PubMed search using the following search terms: psycho* OR prodrom* OR “ultra high risk” OR “at risk mental state” OR “clinical high risk” AND prediction. The search was restricted to peer-reviewed studies published in English from October 2015 to November 2019. In their review, Schmidt et al identified 25 studies that generated models predicting onset of psychosis. Among biological models, the highest positive predictive value (83%) was achieved by a neuroimaging model (grey matter volume reduction on MRI). Schmidt et al also examined the role of sequential testing, finding that the highest positive predictive value was obtained using three models sequentially: a combined model (clinical plus electroencephalography), then structural MRI, followed by blood biomarkers. Since this review there have been several further studies describing predictive models for outcomes in CHR. These have incorporated data (or combinations of data) from several different modalities including socio-demographic, clinical, neuroimaging, linguistic and biological sources. With regard to prediction of development of psychosis, published models vary with regard to their predictive performance, with area under the receiver-operating curve typically in the range 0.60-0.90.

Added Value of this Study

In this study, we report the development of prediction models using support vector machine learning techniques based on proteomic data obtained from mass spectrometry of baseline plasma samples. We found that the clinical variables included in our study did not usefully predict development of psychosis. Proteomic data were highly predictive, and a model based on the 10 most predictive proteins performed well for prediction of transition outcome in training and test data. Proteomic data also contributed to prediction of functional outcome, though with less accuracy in comparison to transition outcome. Analysis of differentially expressed proteins provided particular evidence for implication of the complement and coagulation cascades. We also developed a prediction model based on proteomic data in a general population sample, with several of the same proteins weighted highly for prediction of outcomes in both samples.

### Implications of all the Available Evidence

Proteomic features may helpfully contribute to outcome prediction in CHR individuals, and in particular for prediction of development of psychosis. However, the models we have developed require validation in external samples to assess their validity and applicability in the clinical setting.

### TABLES

TABLE 1
Descriptive statistics for EU-GEI CHR-T and CHR-NT groups

| | Missing | | | | | | | |
|---|---|---|---|---|---|---|---|---|
| | data, | | CHR-T | | CHR-NT | | | |
| | n (%) | | N = 49 | | N = 84 | | t/χ² | p |
| Baseline age | 0 | | 22.2 | (5.0) | 22.9 | (4.2) | −0.824 | 0.412 |
| in years, | | | | | | | | |
| mean (SD) | | | | | | | | |
| Sex, n (%) | 0 | | 26 male | (53.1%) | 42 male | (50.0%) | 0.116 | 0.733 |
| | | | 23 female | (46.9%) | 42 female | (50.0%) | | |
| Baseline BMI | 20 | (15.0%) | 24.5 | (4.5) | 24.4 | (6.1) | 0.116 | 0.908 |
| in kg/m², | | | | | | | | |
| mean (SD) | | | | | | | | |
| Baseline years | 14 | (10.5%) | 14.1 | (3.4) | 14.4 | (3.0) | −0.625 | 0.533 |
| in education, | | | | | | | | |
| mean (SD) | | | | | | | | |
| Ethnicity, | 0 | | 33 white | (67.3%) | 58 white | (69.0%) | 2.370 | 0.306 |
| n (%) | | | 8 black | (16.3%) | 7 black | (8.3%) | | |
| | | | 8 other | (16.3%) | 19 other | (22.6%) | | |
| Ever used | 3 | (2.3%) | 36 yes | (73.5%) | 65 yes | (77.4%) | 0.051 | 0.821 |
| cannabis, | | | 11 no | (22.4%) | 18 no | (21.4%) | | |
| n (%) | | | 2 not known | (4.1%) | 1 not known | (1.2%) | | |
| Baseline | 29 | (21.8%) | 15 yes | (30.6%) | 26 yes | (31.0%) | 0.030 | 0.862 |
| cannabis | | | 22 no | (44.9%) | 41 no | (48.8%) | | |
| use, n (%) | | | 12 not known | (24.5%) | 17 not known | (20.2%) | | |
| Baseline | 14 | (10.5%) | 21 yes | (42.9%) | 43 yes | (51.2%) | 0.373 | 0.541 |
| tobacco | | | 21 no | (42.9%) | 34 no | (40.5%) | | |
| use, n (%) | | | 7 not known | (14.2%) | 7 not known | (8.3%) | | |
| Baseline | 3 | (2.3%) | 35 yes | (71.4%) | 58 yes | (69.0%) | 0.071 | 0.790 |
| alcohol use, | | | 13 no | (26.5%) | 24 no | (28.6%) | | |
| n (%) | | | 1 not known | (2.0%) | 2 not known | (2.4%) | | |
| Baseline | 31 | (23.3%) | 19 yes | (38.8%) | 32 yes | (38.1%) | 0.042 | 0.839 |
| medication | | | Antidepressant | 12 | Antidepressant | 17 | | |
| use, n (%) | | | Antipsychotic | 5 | Antipsychotic | 4 | | |
| | | | Hypnotic | 1 | Hypnotic | 6 | | |
| | | | Other | 1 | Other | 5 | | |
| | | | 20 no | (40.8%) | 31 no | (36.9%) | | |
| | | | 10 not known | (20.4%) | 21 not known | (25.0%) | | |
| Baseline GAF | 12 | (9.0%) | 52.4 | (10.3) | 56.0 | (10.0) | −1.906 | 0.059 |
| symptoms | | | | | | | | |
| score, mean | | | | | | | | |
| (SD) | | | | | | | | |
| Baseline GAF | 5 | (3.8%) | 52.3 | (12.4) | 54.8 | (11.3) | −1.148 | 0.253 |
| disability | | | | | | | | |
| score, mean | | | | | | | | |
| (SD) | | | | | | | | |
| Baseline SANS | 19 | (14.3%) | 20.9 | (14.0) | 16.2 | (11.6) | 1.903 | 0.060 |
| total | | | | | | | | |
| composite | | | | | | | | |
| score, mean | | | | | | | | |
| (SD) | | | | | | | | |
| Baseline SANS | 11 | (8.3%) | 6.6 | (4.1) | 5.8 | (3.7) | 1.158 | 0.249 |
| total global | | | | | | | | |
| score, mean | | | | | | | | |
| (SD) | | | | | | | | |
| Baseline BPRS | 10 | (7.5%) | 49.1 | (11.5) | 44.2 | (10.2) | 2.452 | 0.016 |
| total score, | | | | | | | | |
| mean (SD) | | | | | | | | |
| Baseline | 7 | (5.3%) | 20.3 | (10.4) | 19.2 | (9.2) | 0.657 | 0.512 |
| MADRS total | | | | | | | | |
| score, mean | | | | | | | | |
| (SD) | | | | | | | | |
| 2 year GAF | 62 | (46.7%) | 42.3 | (13.2) | 62.2 | (10.3) | −7.125 | <0.001 |
| symptoms | | | | | | | | |
| score, mean | | | | | | | | |
| (SD) ^{a} | | | | | | | | |
| 2 year GAF | 54 | (40.6%) | 44.7 | (9.1) | 64.5 | (12.8) | −8.024 | <0.001 |
| disability | | | | | | | | |
| score, mean | | | | | | | | |
| (SD) ^{b} | | | | | | | | |
| 2 year GAF | 54 | (40.6%) | 29 poor | (59.2%) | 18 poor | (21.4%) | 27.734 | <0.001 |
| disability | | | functioning | | functioning | | | |

| | | | | |
|---|---|---|---|---|
| score, | 1 good | (2.0%) | 31 good | (36.9%) |
| dichotomous | functioning | | functioning | |
| outcome ^{c} | 19 not known | (38.8%) | 35 not known | (41.7%) |

^{a} Data available for 71 of 133 participants (CHR-NT n = 44, CHR-T n = 27)
^{b} Data available for 79 of 133 participants (CHR-NT n = 49, CHR-T n = 29)
^{c} Poor functioning: GAF disability score ≤60; good functioning: GAF disability score >60
Tobacco use was defined as daily use for at least 1 month over the previous 12 months.
Alcohol use was defined as at least 12 or more alcoholic beverages over the previous 12 months.
Missing data excluded in hypothesis tests.

EU-GEI: European Network of National Schizophrenia Networks Studying Gene-Environment Interactions; CHR-T: clinical high risk, transitioned to psychosis; CHR-NT: clinical high risk, did not transition to psychosis; BMI: body mass index; GAF: General Assessment of Functioning; SANS: Scale for the Assessment of Negative Symptoms; BPRS: Brief Psychiatric Rating Scale; MADRS: Montgomery Asberg Depression Rating Scale

TABLE 2
Performance metrics for support vector machine models

| | | | | | | | Bal- | | | | | |
|---|---|---|---|---|---|---|---|---|---|---|---|---|
| | True | False | True | False | | | anced | AUC | Positive | Negative | Positive | Negative |
| | posi- | nega- | nega- | posi- | Sensi- | Speci- | accu- | (95% | predic- | predic- | likeli- | likeli- |
| | tives, | tives, | tives, | tives, | tivity, | ficity, | racy, | confidence | tive | tive | hood | hood, |
| Model description | n (%) | n (%) | n (%) | n (%) | % | % | % | interval) | value, % | value, % | ratio | ratio |
| Model 1a: clinical | 45 | 4 | 74 | 10 | 91.8 | 88.1 | 90.0 | 0.96 | 81.8 | 94.9 | 7.7 | 0.1 |
| and proteomic | (92%) | (8%) | (88%) | (12%) | | | | (0.92-1.00) | | | | |
| Dataset: EU-GEI | | | | | | | | | | | | |
| Features: 65 clinical | | | | | | | | | | | | |
| and 166 proteomic | | | | | | | | | | | | |
| Target: transition | | | | | | | | | | | | |
| status | | | | | | | | | | | | |
| N: 49 transition, 84 | | | | | | | | | | | | |
| non-transition | | | | | | | | | | | | |
| Model 1b: clinical | 22 | 27 | 40 | 44 | 44.9 | 47.6 | 46.3 | 0.47 | 33.3 | 59.7 | 0.9 | 1.2 |
| Dataset: EU-GEI | (45%) | (55%) | (48%) | (52%) | | | | (0.37-0.57) | | | | |
| Features: 65 clinical | | | | | | | | | | | | |
| Target: transition | | | | | | | | | | | | |
| status | | | | | | | | | | | | |
| N: 49 transition, 84 | | | | | | | | | | | | |
| non-transition | | | | | | | | | | | | |
| Model 1c: proteomic | 45 | 4 | 75 | 9 | 91.8 | 89.3 | 90.6 | 0.97 | 83.3 | 94.9 | 8.6 | 0.1 |
| Dataset: EU-GEI | (92%) | (8%) | (89%) | (11%) | | | | (0.94-1.00) | | | | |
| Features: 166 | | | | | | | | | | | | |
| proteomic | | | | | | | | | | | | |
| Target: transition | | | | | | | | | | | | |
| status | | | | | | | | | | | | |
| N: 49 transition, 84 | | | | | | | | | | | | |
| non-transition | | | | | | | | | | | | |
| Model 1d: top 10, | 28 | 2 | 45 | 5 | 93.3 | 90.0 | 91.7 | 0.97 | 84.8 | 95.7 | 9.3 | 0.1 |
| training | (93%) | (7%) | (90%) | (10%) | | | | (0.93-1.00) | | | | |
| Dataset: EU-GEI, | | | | | | | | | | | | |
| London data withheld | | | | | | | | | | | | |
| Features: 10 proteomic | | | | | | | | | | | | |
| Target: transition | | | | | | | | | | | | |
| status | | | | | | | | | | | | |
| N: 30 transition, 50 | | | | | | | | | | | | |
| non-transition | | | | | | | | | | | | |
| Model 1d: top 10, test | 16 | 3 | 30 | 4 | 84.2 | 88.2 | 86.2 | 0.93 | 80.0 | 90.9 | 7.2 | 0.2 |
| Dataset: EU-GEI, | (84%) | (16%) | (88%) | (12%) | | | | (0.85-1.00) | | | | |
| London data | | | | | | | | | | | | |
| Features: 10 proteomic | | | | | | | | | | | | |
| Target: transition | | | | | | | | | | | | |
| status | | | | | | | | | | | | |
| N: 19 transition, 34 | | | | | | | | | | | | |
| non-transition | | | | | | | | | | | | |
| Model 2: ELISA | 31 | 13 | 54 | 28 | 70.5 | 65.9 | 68.2 | 0.76 | 52.5 | 80.6 | 2.1 | 0.4 |
| Dataset: EU-GEI | (70%) | (30%) | (66%) | (34%) | | | | (0.67-0.85) | | | | |
| Features: 9 ELISA | | | | | | | | | | | | |
| Target: transition | | | | | | | | | | | | |
| status | | | | | | | | | | | | |
| N: 44 transition, 82 | | | | | | | | | | | | |
| non-transition | | | | | | | | | | | | |
| Model 3: functional | 25 | 22 | 20 | 12 | 53.2 | 62.5 | 57.8 | 0.72 | 67.6 | 47.6 | 1.4 | 0.7 |
| outcome | (53%) | (47%) | (63%) | (37%) | | | | (0.61-0.83) | | | | |
| Dataset: EU-GEI | | | | | | | | | | | | |
| Features: 65 clinical | | | | | | | | | | | | |
| and 166 proteomic | | | | | | | | | | | | |
| Target: functional | | | | | | | | | | | | |
| outcome | | | | | | | | | | | | |
| N: 47 poor | | | | | | | | | | | | |
| functioning, 32 good | | | | | | | | | | | | |
| functioning | | | | | | | | | | | | |
| Model 4: replication | 47 | 2 | 82 | 4 | 95.9 | 95.3 | 95.6 | 0.98 | 92.2 | 97.6 | 20.6 | <0.1 |
| Dataset: EU-GEI | (96%) | (4%) | (95%) | (5%) | | | | (0.95-1.00) | | | | |
| replication | | | | | | | | | | | | |
| Features: 65 clinical | | | | | | | | | | | | |
| and 119 proteomic | | | | | | | | | | | | |
| Target: transition | | | | | | | | | | | | |
| status | | | | | | | | | | | | |
| N: 49 transition, 86 | | | | | | | | | | | | |
| non-transition | | | | | | | | | | | | |
| Model 5: ALSPAC | 38 | 17 | 49 | 17 | 69.1 | 74.2 | 71.7 | 0.76 | 69.1 | 74.2 | 2.7 | 0.4 |
| PEs | (69%) | (31%) | (74%) | (26%) | | | | (0.67-0.85) | | | | |
| Dataset: ALSPAC | | | | | | | | | | | | |
| Features: 265 proteomic | | | | | | | | | | | | |
| Target: PEs age 18 | | | | | | | | | | | | |
| N: 55 PEs, 66 no PE | | | | | | | | | | | | |

AUC: area under the receiver-operating characteristic curve; EU-GEI: European Network of National Schizophrenia Networks Studying Gene-Environment Interactions; ALSPAC: Avon Longitudinal Study of Parents and Children; PEs: psychotic experiences. Models 1a-d, 2 and 3 are adjusted for age, sex, body mass index and years in education and Model 4 is additionally adjusted for ethnicity and tobacco use. Model 5 is adjusted for sex, maternal social class at birth and body mass index at age 12.

TABLE 3
Ten percent highest-weighted features for support vector machine models (ranked
according to mean feature weight for models selected in cross-validation inner loop)

| Model 1a: EU-GEI transition | | Model 3: EU-GEI functional outcome | | Model 5: ALSPAC psychotic experiences | |
|---|---|---|---|---|---|
| | Mean | | Mean | | Mean |
| Feature | weight | Feature | weight | Feature | weight |
| P01023 Alpha-2-macroglobulin | −0.339 | P01023 Alpha-2- | −0.180 | P04003 C4b-binding protein | −0.228 |
| P01871 Immunoglobulin heavy | −0.242 | macroglobulin | | alpha chain | |
| constant mu | | BPRS: suspiciousness | 0.179 | P27169 Serum paraoxonase/ | −0.182 |
| P04003 C4b-binding protein | −0.154 | P55058 Phospholipid | −0.178 | arylesterase 1 | |
| alpha chain | | transfer protein | | P07225 Vitamin K-dependent | −0.150 |
| P55058 Phospholipid | −0.153 | P01871 Immunoglobulin | −0.167 | protein S | |
| transfer protein | | heavy constant mu | | Q03591 Complement factor | −0.147 |
| P07357 Complement component | 0.152 | O43866 CDS antigen-like | −0.164 | H-related protein 1 | |
| 8 alpha chain | | Q9UGM5 Fetuin-B | 0.147 | P61626 Lysozyme C | −0.145 |
| P07225 Vitamin K-dependent- | −0.139 | P19827 Inter-alpha-trypsin | 0.133 | P55103 Inhibin beta C chain | 0.132 |
| protein S | | inhibitor heavy chain H1 | | Q08380 Galectin-3-binding | 0.131 |
| O75636 Ficolin-3 | −0.139 | P14618 Pyruvate kinase | −0.128 | protein | |
| P02766 Transthyretin | −0.138 | SANS: impersistence at | 0.122 | P01871 Immunoglobulin heavy | −0.128 |
| P13671 Complement component 6 | 0.128 | work or school | | constant mu | |
| P02774 Vitamin D binding protein | 0.120 | SANS: increased latency | 0.115 | P01019 Angiotensinogen | −0.123 |
| P43320 Beta-crystallin B2 | 0.120 | of response | | P24593 Insulin-like growth | 0.122 |
| P02753 Retinol-binding protein 4 | 0.120 | SANS: blocking | 0.110 | factor-binding protein 5 | |
| P23142 Fibulin-1 | 0.113 | P17936 insulin-like growth | 0.102 | P00746 Complement factor D | 0.120 |
| P10909 Clusterin | 0.112 | factor-binding protein 3 | | P09871 Complement C1s | −0.115 |
| P19827 Inter-alpha-trypsin | 0.112 | P10909 Clusterin | 0.099 | subcomponent | |
| inhibitor heavy chain H1 | | SANS: grooming and hygiene | 0.099 | P02654 Apolipoprotein C-I | −0.113 |
| P05155 Plasma protease | −0.112 | Q08380 Galectin-3- | 0.096 | O75636 Ficolin-3 | 0.113 |
| C1 inhibitor | | binding protein | | Q9NQ79 Cartilage acidic protein 1 | 0.112 |
| P08697 Alpha-2-antiplasmin | −0.111 | P36955 Pigment epithelium- | 0.095 | P01023 Alpha-2-macroglobulin | −0.109 |
| MADRS: concentration difficulties | −0.104 | derived factor | | P10909 Clusterin | −0.108 |
| P02747 Complement C1q | 0.103 | P01042 Kininogen-1 | 0.092 | P04275 von Willebrand factor | −0.107 |
| subcomponent subunit C | | P60174 Triosephosphate | −0.092 | P07358 Complement component | 0.101 |
| P07195 L-lactate dehydrogenase | 0.103 | isomerase | | C8 beta chain | |
| B chain | | BPRS: excitement | 0.091 | Q5T7F0 Neuropilin | −0.100 |
| Q16610 Extracellular matrix | −0.102 | P02656 Apolipoprotein C-III | 0.091 | Q9H4A9 Dipeptidase 2 | −0.100 |
| protein 1 | | SANS: sexual activity | −0.090 | P02679 Fibrinogen gamma chain | −0.100 |
| P02489 Alpha-crystallin A chain | 0.101 | MADRS: suicidal thoughts | −0.090 | P24592 Insulin-like growth factor- | 0.099 |
| Q76LX8 A disintegrin and | −0.100 | P04275 von Willebrand factor | 0.089 | binding protein 6 | |
| metalloproteinase with | | | | O95497 Pantetheinase | 0.098 |
| thrombospondin motifs 13 | | | | P04040 Catalase | 0.098 |
| | | | | H0Y755 Low affinity | 0.096 |
| | | | | immunoglobulin gamma Fc | |
| | | | | region receptor III-A | |

Proteins are presented with their Uniprot accession number and corresponding protein name.
EU-GEI: European Network of National Schizophrenia Networks Studying Gene-Environment Interactions; ALSPAC: Avon Longitudinal Study of Parents and Children; BPRS: Brief Psychiatric Rating Scale; MADRS: Montgomery-Asberg Depression Rating Scale; SANS: Scale for the Assessment of Negative Symptoms

### REFERENCES

1. Larsen T K, Melle I, Auestad B, Haahr U, Joa I, Johannessen J O, Opjordsmoen S, Rund B R, Rossberg J I, Simonsen E, Vaglum P, Friis S, McGlashan T. Early detection of psychosis: positive effects on 5-year outcome. Psychol Med. 2011; 41(7):1461-1469.
2. Fusar-Poli P, Borgwardt S, Bechdolf A, Addington J, Riecher-Rössler A, Schultze-Lutter F, Keshavan M, Wood S, Ruhrmann S, Seidman Li, Valmaggia L, Cannon T, Velthorst E, De Haan L, Cornblatt B, Bonoldi I, Birchwood M, McGlashan T, Carpenter W, McGorry P, Klosterkötter J, McGuire P, Yung A. The psychosis high-risk state: a comprehensive state-of-the-art review. JAMA psychiatry. 2013; 70(1):107-120.
3. Schmidt S J, Schultze-Lutter F, Schimmelmann B G, Maric N P, Salokangas R K, Riecher-Rossler A, van der Gaag M, Meneghelli A, Nordentoft M, Marshall M, Morrison A, Raballo A, Klosterkotter J, Ruhrmann S. EPA guidance on the early intervention in clinical high risk states of psychoses. Eur Psychiatry. 2015; 30(3):388-404.
4. Schultze-Lutter F, Michel C, Schmidt Si, Schimmelmann B G, Maric N P, Salokangas R K, Riecher-Rossler A, van der Gaag M, Nordentoft M, Raballo A, Meneghelli A, Marshall M, Morrison A, Ruhrmann S, Klosterkotter J. EPA guidance on the early detection of clinical high risk states of psychoses. Eur Psychiatry. 2015; 30(3):405-416.
5. Fusar-Poli P, Bonoldi I, Yung A R, Borgwardt S, Kempton M J, Valmaggia L, Barale F, Caverzasi E, McGuire P. Predicting psychosis: meta-analysis of transition outcomes in individuals at high clinical risk. Arch Gen Psychiatry. 2012; 69(3):220-229.
6. Cannon T D, Yu C, Addington J, Bearden C E, Cadenhead K S, Cornblatt B A, Heinssen R, Jeffries C D, Mathalon D H, McGlashan T H, Perkins D O, Seidman U, Tsuang M T, Walker E F, Woods S W, Kattan M W. An Individualized Risk Calculator for Research in Prodromal Psychosis. Am J Psychiatry. 2016; 173(10):980-988.
7. Rutigliano G, Valmaggia L, Landi P, Frascarelli M, Cappucciati M, Sear V, Rocchetti M, De Micheli A, Jones C, Palombini E, McGuire P, Fusar-Poli P. Persistence or recurrence of non-psychotic comorbid mental disorders associated with 6-year poor functional outcomes in patients at ultra high risk for psychosis. J Affect Disord. 2016; 203:101-110.
8. Kelleher I, Keeley H, Corcoran P, Lynch F, Fitzpatrick C, Devlin N, Molloy C, Roddy S, Clarke M C, Harley M, Arseneault L, Wasserman C, Carli V, Sarchiapone M, Hoven C, Wasserman D, Cannon M. Clinicopathological significance of psychotic experiences in non-psychotic young people: evidence from four population-based studies. Br J Psychiatry. 2012; 201(1):26-32.
9. Fusar-Poli P, Rocchetti M, Sardella A, Avila A, Brandizzi M, Caverzasi E, Politi P, Ruhrmann S, McGuire P. Disorder, not just state of risk: meta-analysis of functioning and quality of life in people at high risk of psychosis. Br J Psychiatry. 2015; 207(3):198-206.
10. Addington J, Cornblatt B A, Cadenhead K S, Cannon T D, McGlashan T H, Perkins D O, Seidman U, Tsuang M T, Walker E F, Woods S W, Heinssen R. At clinical high risk for psychosis: outcome for nonconverters. Am J Psychiatry. 2011; 168(8):800-805.
11. Zammit S, Kounali D, Cannon M, David A S, Gunnell D, Heron J, Jones P B, Lewis S, Sullivan S, Wolke D, Lewis G. Psychotic experiences and psychotic disorders at age 18 in relation to psychotic experiences at age 12 in a longitudinal population-based cohort study. Am J Psychiatry. 2013; 170(7):742-750.
12. Healy C, Brannigan R, Dooley N, Coughlan H, Clarke M, Kelleher I, Cannon M. Childhood and adolescent psychotic experiences and risk of mental disorder: a systematic review and meta-analysis. Psychol Med. 2019; 49(10):1589-1599.
13. Yates K, Lang U, Cederlof M, Boland F, Taylor P, Cannon M, McNicholas F, DeVylder J, Kelleher I. Association of Psychotic Experiences With Subsequent Risk of Suicidal Ideation, Suicide Attempts, and Suicide Deaths: A Systematic Review and Meta-analysis of Longitudinal Population Studies. JAMA Psychiatry. 2019; 76(2):180-189.
14. Kelleher I, Wigman J T, Harley M, O'Hanlon E, Coughlan H, Rawdon C, Murphy J, Power E, Higgins N M, Cannon M. Psychotic experiences in the population: Association with functioning and mental distress. Schizophr Res. 2015; 165(1):9-14.
15. Healy C, Campbell D, Coughlan H, Clarke M, Kelleher I, Cannon M. Childhood psychotic experiences are associated with poorer global functioning throughout adolescence and into early adulthood. Acta Psychitr Scand. 2018; 138(1):26-34.
16. McGorry P, Keshavan M, Goldstone S, Amminger P, Allott K, Berk M, Lavoie S, Pantelis C, Yung A, Wood S, Hickie I. Biomarkers and clinical staging in psychiatry. World Psychiatry. 2014; 13(3):211-223.
17. Jeffries C D, Perkins D O, Fournier M, Do K Q, Cuenod M, Khadimallah I, Domenici E, Addington J, Bearden C E, Cadenhead K S, Cannon T D, Cornblatt B A, Mathalon D H, McGlashan T H, Seidman L I, Tsuang M, Walker E F, Woods S W. Networks of blood proteins in the neuroimmunology of schizophrenia. Transl Psychiatry. 2018; 8(1):112.
18. Upthegrove R, Manzanares-Teson N, Barnes N M. Cytokine function in medication-naive first episode psychosis: A systematic review and meta-analysis. Schizophrenia Research. 2014; 155(1-3):101-108.
19. Chan M K, Krebs M O, Cox D, Guest P C, Yolken R H, Rahmoune H, Rothermundt M, Steiner J, Leweke F M, van Beveren N J, Niebuhr D W, Weber N S, Cowan D N, Suarez-Pinilla P, Crespo-Facorro B, Mam-Lam-Fook C, Bourgin J, Wenstrup R J, Kaldate R R, Cooper J D, Bahn S. Development of a blood-based molecular biomarker test for identification of schizophrenia before disease onset. Transl Psychiatry. 2015; 5:e601.
20. Sabherwal S, English J A, Focking M, Cagney G, Cotter D R. Blood biomarker discovery in drug-free schizophrenia: the contribution of proteomics and multiplex immunoassays. Expert Rev Proteomics. 2016; 13(12):1141-1155.
21. Schmitt A, Martins-de-Souza D, Akbarian S, Cassoli J S, Ehrenreich H, Fischer A, Fonteh A, Gattaz W F, Gawlik M, Gerlach M, Grunblatt E, Halene T, Hasan A, Hashimoto K, Kim Y K, Kirchner S K, Kornhuber J, Kraus T F J, Malchow B, Nascimento J M, Rossner M, Schwarz M, Steiner J, Talib L, Thibaut F, Riederer P, Falkai P. Consensus paper of the WFSBP Task Force on Biological Markers: Criteria for biomarkers and endophenotypes of schizophrenia, part Ill: Molecular mechanisms. World J Biol Psychiatry. 2017; 18(5):330-356.
22. English J A, Lopez L M, O'Gorman A, Focking M, Hryniewiecka M, Scaife C, Sabherwal S, Wynne K, Dicker P, Rutten B P F, Lewis G, Zammit S, Cannon M, Cagney G, Cotter D R. Blood-Based Protein Changes in Childhood Are Associated With Increased Risk for Later Psychotic Disorder: Evidence From a Nested Case-Control Study of the ALSPAC Longitudinal Birth Cohort. Schizophr Bull. 2018; 44(4:297-306.
23. Ricking M, Sabherwal S, Cates H M, Scaife C, Dicker P, Hryniewiecka M, Wynne K, Rutten B P F, Lewis G, Cannon M, Nestler E J, Heurich M, Cagney G, Zammit S, Cotter D R. Complement pathway changes at age 12 are associated with psychotic experiences at age 18 in a longitudinal population-based study: evidence for a role of stress. Molecular Psychiatry. 2019.
24. Li Y, Zhou K, Zhang Z, Sun L, Yang J, Zhang M, Ji B, Tang K, Wei Z, He G, Gao L, Yang L, Wang P, Yang P, Feng G, He L, Wan C. Label-free quantitative proteomic analysis reveals dysfunction of complement pathway in peripheral blood of schizophrenia patients: evidence for the immune hypothesis of schizophrenia. Mol Biosyst. 2012; 8(10):2664-2671.
25. Sekar A, Bialas A R, de Rivera H, Davis A, Hammond T R, Kamitaki N, Tooley K, Presumey J, Baum M, Van Doren V, Genovese G, Rose S A, Handsaker R E, Daly M J, Carroll M C, Stevens B, McCarroll S A. Schizophrenia risk from complex variation of complement component 4. Nature. 2016; 530(7589):177-183.
26. Zhang C, Zhang D F, Wu Z G, Peng D H, Chen J, Ni J, Tang W, Xu L, Yao Y G, Fang Y R. Complement factor H and susceptibility to major depressive disorder in Han Chinese. Br J Psychiatry. 2016; 208(5):446-452.
27. Koutsouleris N, Kambeitz-llankovic L, Ruhrmann S, Rosen M, Ruef A, Dwyer D B, Paolini M, Chisholm K, Kambeitz J, Haidl T, Schmidt A, Gillam J, Schultze-Lutter F, Falkai P, Reiser M, Riecher-Rossler A, Upthegrove R, Hietala J, Salokangas R K R, Pantelis C, Meisenzahl E, Wood S J, Beque D, Brambilla P, Borgwardt S, Consortium P. Prediction Models of Functional Outcomes for Individuals in the Clinical High-Risk State for Psychosis or With Recent-Onset Depression: A Multimodal, Multisite Machine Learning Analysis. JAMA Psychiatry. 2018; 75(11):1156-1172.
28. Fusar-Poli P, Hijazi Z, Stahl D, Steyerberg E W. The science of prognosis in psychiatry: A review. JAMA Psychiatry. 2018; 75(12):1289-1297.
29. Hahn T, Nierenberg A A, Whitfield-Gabrieli S. Predictive analytics in mental health: applications, guidelines, challenges and perspectives. Molecular Psychiatry. 2016; 22:37.
30. Jongsma H E, Gayer-Anderson C, Lasalvia A, Quattrone D, Mule A, Szoke A, Selten J P, Turner C, Arango C, Tarricone I, Berardi D, Tortelli A, Llorca P M, de Haan L, Bobes J, Bernardo M, Sanjuan J, Santos J L, Arrojo M, Del-Ben C M, Menezes P R, Velthorst E, Murray R M, Rutten B P, Jones P B, van Os J, Morgan C, Kirkbride J B, European Network of National Schizophrenia Networks Studying Gene-Environment Interactions Work Package G. Treated Incidence of Psychotic Disorders in the Multinational E U-GEI Study. JAMA Psychiatry. 2018; 75(1):36-46.
31. European Network of National Networks studying Gene-Environment Interactions in S, van Os J, Rutten B P, Myin-Germeys I, Delespaul P, Viechtbauer W, van Zelst C, Bruggeman R, Reininghaus U, Morgan C, Murray R M, Di Forti M, McGuire P, Valmaggia L R, Kempton M J, Gayer-Anderson C, Hubbard K, Beards S, Stilo S A, Onyejiaka A, Bourque F, Modinos G, Tognin S, Calem M, O'Donovan M C, Owen M J, Holmans P, Williams N, Craddock N, Richards A, Humphreys I, Meyer-Lindenberg A, Leweke F M, Tost H, Akdeniz C, Rohleder C, Bumb J M, Schwarz E, Alptekin K, Ucok A, Saka M C, Atbasoglu E C, Guloksuz S, Gumus-Akay G, Cihan B, Karadag H, Soygur H, Cankurtaran E S, Ulusoy S, Akdede B, Binbay T, Ayer A, Noyan H, Karadayi G, Akturan E, Ulas H, Arango C, Parellada M, Bernardo M, Sanjuan J, Bobes J, Arrojo M, Santos J L, Cuadrado P, Rodriguez Solano J J, Carracedo A, Garcia Bernardo E, Roldan L, Lopez G, Cabrera B, Cruz S, Diaz Mesa E M, Pouso M, Jimenez E, Sanchez T, Rapado M, Gonzalez E, Martinez C, Sanchez E, Olmeda M S, de Haan L, Velthorst E, van der Gaag M, Selten J P, van Dam D, van der Ven E, van der Meer F, Messchaert E, Kraan T, Burger N, Leboyer M, Szoke A, Schurhoff F, Llorca P M, Jamain S, Tortelli A, Frijda F, Vilain J, Galliot A M, Baudin G, Ferchiou A, Richard J R, Bulzacka E, Charpeaud T, Tronche A M, De Hert M, van Winkel R, Decoster J, Derom C, Thiery E, Stefanis N C, Sachs G, Aschauer H, Lasser I, Winklbaur B, Schlogelhofer M, Riecher-Rossler A, Borgwardt S, Walter A, Harrisberger F, Smieskova R, Rapp C, Ittig S, Soguel-dit-Piquard F, Studerus E, Klosterkotter J, Ruhrmann S, Paruch J, Julkowski D, Hilboll D, Sham P C, Cherny S S, Chen E Y, Campbell D D, Li M, Romeo-Casabona C M, Emaldi Cirion A, Urruela Mora A, Jones P, Kirkbride J, Cannon M, Rujescu D, Tarricone I, Berardi D, Bonora E, Seri M, Marcacci T, Chiri L, Chierzi F, Storbini V, Braca M, Minenna M G, Donegani I, Fioritti A, La Barbera D, La Cascia C E, Mule A, Sideli L, Sartorio R, Ferraro L, Tripoli G, Seminerio F, Marinaro A M, McGorry P, Nelson B, Amminger G P, Pantelis C, Menezes P R, Del-Ben C M, Gallo Tenan S H, Shuhama R, Ruggeri M, Tosato S, Lasalvia A, Bonetto C, Ira E, Nordentoft M, Krebs M O, Barrantes-Vidal N, Cristobal P, Kwapil T R, Brietzke E, Bressan R A, Gadelha A, Maric N P, Andric S, Mihaljevic M, Mirjanic T. Identifying gene-environment interactions in schizophrenia: contemporary challenges for integrated, large-scale investigations. Schizophr Bull. 2014; 40(4):729-736.
32. Kraan T C, Velthorst E, Themmen M, Valmaggia L, Kempton M J, McGuire P, van Os J, Rutten B P F, Smit F, de Haan L, van der Gaag M, Study E-GHR. Child Maltreatment and Clinical Outcome in Individuals at Ultra-High Risk for Psychosis in the EU-GEI High Risk Study. Schizophr Bull. 2018; 44(3):584-592.
33. Yung A R, Yuen H P, McGorry P D, Phillips U, Kelly D, Dell'Olio M, Francey S M, Cosgrave E M, Killackey E, Stanford C, Godfrey K, Buckby J. Mapping the onset of psychosis: the Comprehensive Assessment of At-Risk Mental States. Aust N Z J Psychiatry. 2005; 39(11-12):964-971.
34. Aas I H. Global Assessment of Functioning (GAF): properties and frontier of current knowledge. Ann Gen Psychiatry. 2010; 9:20.
35. Goldman H H, Skodol A E, Lave T R. Revising axis V for DSM-IV: a review of measures of social functioning. Am J Psychiatry. 1992; 149(9):1148-1156.
36. Koutsouleris N, Kambeitz-llankovic L, Ruhrmann S, Rosen M, Ruef A, Dwyer D B, Paolini M, Chisholm K, Kambeitz J, Haidl T, Schmidt A, Gillam J, Schultze-Lutter F, Falkai P, Reiser M, Riecher-Rossler A, Upthegrove R, Hietala J, Salokangas R K R, Pantelis C, Meisenzahl E, Wood S J, Beque D, Brambilla P, Borgwardt S. Prediction Models of Functional Outcomes for Individuals in the Clinical High-Risk State for Psychosis or With Recent-Onset Depression: A Multimodal, Multisite Machine Learning Analysis. JAMA Psychiatry. 2018; 75(11):1156-1172.
37. Andreasen N C. The Scale for the Assessment of Negative Symptoms (SANS): conceptual and theoretical foundations. Br J Psychiatry Suppl. 1989(7):49-58.
38. Overall J E, Gorham D R. The Brief Psychiatric Rating Scale. Psychological Reports. 1962; 10(3):799-812.
39. Montgomery S A, Asberg M. A new depression scale designed to be sensitive to change. Br J Psychiatry. 1979; 134:382-389.
40. English J A, Fan Y, Focking M, Lopez L M, Hryniewiecka M, Wynne K, Dicker P, Matigian N, Cagney G, Mackay-Sim A, Cotter D R. Reduced protein synthesis in schizophrenia patient-derived olfactory cells. Transl Psychiatry. 2015; 5:e663.
41. Focking M, Opstelten R, Prickaerts J, Steinbusch H W, Dunn M J, van den Hove D L, Cotter D R. Proteomic investigation of the hippocampus in prenatally stressed mice implicates changes in membrane trafficking, cytoskeletal, and metabolic function. Dev Neurosci. 2014; 36(5):432-442.
42. Topol A, Zhu S, Hartley B J, English J, Hauberg M E, Tran N, Rittenhouse C A, Simone A, Ruderfer D M, Johnson J, Readhead B, Hadas Y, Gochman P A, Wang Y C, Shah H, Cagney G, Rapoport J, Gage F H, Dudley J T, Sklar P, Mattheisen M, Cotter D, Fang G, Brennand K J. Dysregulation of miRNA-9 in a Subset of Schizophrenia Patient-Derived Neural Progenitor Cells. Cell Rep. 2016; 15(5):1024-1036.
43. Boyd A, Golding J, Macleod J, Lawlor D A, Fraser A, Henderson J, Molloy L, Ness A, Ring S, Davey Smith G. Cohort Profile: the ‘children of the 90s’—the index offspring of the Avon Longitudinal Study of Parents and Children. Int J Epidemiol. 2013; 42(1):111-127.
44. Fraser A, Macdonald-Wallis C, Tilling K, Boyd A, Golding J, Davey Smith G, Henderson J, Macleod J, Molloy L, Ness A, Ring S, Nelson S M, Lawlor D A. Cohort Profile: the Avon Longitudinal Study of Parents and Children: ALSPAC mothers cohort. Int J Epidemiol. 2013; 42(1):97-110.
45. Cox J, Mann M. Quantitative, high-resolution proteomics for data-driven systems biology. Annu Rev Biochem. 2011; 80:273-299.
46. Cox J, Mann M. MaxQuant enables high peptide identification rates, individualized p.p.b.-range mass accuracies and proteome-wide protein quantification. Nat Biotechnol. 2008; 26(12):1367-1372.
47. Lazar C. Impute LCMD. R package version 2.0. 2015.
48. Benjamini Y, Hochberg Y. Controlling the False Discovery Rate: a Practical and Powerful Approach to Multiple Testing. Journal of the Royal Statistical Society. 1995; Series B(57,1):289-300.
49. Gaonkar B, Davatzikos C. Analytic estimation of statistical significance maps for support vector machine based multi-variate image analysis and classification. NeuroImage. 2013; 78:270-283.
50. Koutsouleris N, Kahn R S, Chekroud A M, Leucht S, Falkai P, Wobrock T, Derks E M, Fleischhacker W W, Hasan A. Multisite prediction of 4-week and 52-week treatment outcomes in patients with first-episode psychosis: a machine learning approach. The Lancet Psychiatry. 2016; 3(10):935-946.
51. Golland P, Fischl B. Permutation Tests for Classification: Towards Statistical Significance in Image-Based Studies. Paper presented at: Information Processing in Medical Imaging; 2003//, 2003; Berlin, Heidelberg.
52. Fan R-E, Chang K-W, Hsieh C-J, Wang X-R, Lin C-J. LIBLINEAR: A Library for Large Linear Classification. J. Mach. Learn. Res. 2008; 9:1871-1874.
53. Krawczyk B. Learning from imbalanced data: open challenges and future directions. Progress in Artificial Intelligence. 2016; 5(4):221-232.
54. Szklarczyk D, Franceschini A, Wyder S, Forslund K, Heller D, Huerta-Cepas J, Simonovic M, Roth A, Santos A, Tsafou K P, Kuhn M, Bork P, Jensen U, von Mering C. STRING v10: protein-protein interaction networks, integrated over the tree of life. Nucleic Acids Res. 2015; 43(Database issue):D447-452.
55. Radua J, Ramella-Cravaro V, Ioannidis J P A, Reichenberg A, Phiphopthatsanee N, Amir T, Yenn Thoo H, Oliver D, Davies C, Morgan C, McGuire P, Murray R M, Fusar-Poli P. What causes psychosis? An umbrella review of risk and protective factors. World Psychiatry. 2018; 17(1):49-66.
56. Studerus E, Ramyead A, Riecher-Rossler A. Prediction of transition to psychosis in patients with a clinical high risk for psychosis: a systematic review of methodology and reporting. Psychological Medicine. 2017; 47(7):1163-1178.
57. Malda A, Boonstra N, Barf H, de Jong S, Aleman A, Addington J, Pruessner M, Nieman D, de Haan L, Morrison A, Riecher-Rossler A, Studerus E, Ruhrmann S, Schultze-Lutter F, An SK, Koike S, Kasai K, Nelson B, McGorry P, Wood S, Lin A, Yung A Y, Kotlicka-Antczak M, Armando M, Vicari S, Katsura M, Matsumoto K, Durston S, Ziermans T, Wunderink L, Ising H, van der Gaag M, Fusar-Poli P, Pijnenborg G H M. Individualized Prediction of Transition to Psychosis in 1,676 Individuals at Clinical High Risk: Development and Validation of a Multivariable Prediction Model Based on Individual Patient Data Meta-Analysis. Front Psychiatry. 2019; 10:345.
58. Riecher-Rossler A, Studerus E. Prediction of conversion to psychosis in individuals with an at-risk mental state: a brief update on recent developments. Curr Opin Psychiatry. 2017; 30(3):209-219.
59. Mechelli A, Lin A, Wood S, McGorry P, Amminger P, Tognin S, McGuire P, Young J, Nelson B, Yung A. Using clinical information to make individualized prognostic predictions in people at ultra high risk for psychosis. Schizophr Res. 2017; 184:32-38.
60. Schmidt A, Cappucciati M, Radua J, Rutigliano G, Rocchetti M, Dell'Osso L, Politi P, Borgwardt S, Reilly T, Valmaggia L, McGuire P, Fusar-Poli P. Improving Prognostic Accuracy in Subjects at Clinical High Risk for Psychosis: Systematic Review of Predictive Models and Meta-analytical Sequential Testing Simulation. Schizophr Bull. 2017; 43(2):375-388.
61. Ruhrmann S, Schultze-Lutter F, Salokangas R K, Heinimaa M, Linszen D, Dingemans P, Birchwood M, Patterson P, Juckel G, Heinz A, Morrison A, Lewis S, von Reventlow H G, Klosterkotter J. Prediction of psychosis in adolescents and young adults at high risk: results from the prospective European prediction of psychosis study. Arch Gen Psychiatry. 2010; 67(3):241-251.
62. Koutsouleris N, Meisenzahl E M, Davatzikos C, Bottlender R, Frodl T, Scheuerecker J, Schmitt G, Zetzsche T, Decker P, Reiser M, Moller R J, Gaser C. Use of neuroanatomical pattern classification to identify subjects in at-risk mental states of psychosis and predict disease transition. Arch Gen Psychiatry. 2009; 66(7):700-712.
63. Koutsouleris N, Borgwardt S, Meisenzahl E M, Bottlender R, Moller H-J, Riecher-Rossler A. Disease Prediction in the At-Risk Mental State for Psychosis Using Neuroanatomical Biomarkers: Results From the FePsy Study. Schizophrenia Bulletin. 2011; 38(6):1234-1246.
64. Das T, Borgwardt S, Hauke D J, Harrisberger F, Lang U E, Riecher-Rossler A, Palaniyappan L, Schmidt A. Disorganized Gyrification Network Properties During the Transition to Psychosis. JAMA Psychiatry. 2018; 75(6):613-622.
65. Koutsouleris N, Davatzikos C, Bottlender R, Patschurek-Kliche K, Scheuerecker J, Decker P, Gaser C, Moller H-J, Meisenzahl E M. Early Recognition and Disease Prediction in the At-Risk Mental States for Psychosis Using Neurocognitive Pattern Classification. Schizophrenia Bulletin. 2011; 38(6):1200-1215.
66. Perkins D O, Jeffries C D, Addington J, Bearden C E, Cadenhead K S, Cannon T D, Cornblatt B A, Mathalon D H, McGlashan T H, Seidman U, Tsuang M T, Walker E F, Woods S W, Heinssen R. Towards a psychosis risk blood diagnostic for persons experiencing high-risk symptoms: preliminary results from the NAPLS project. Schizophr Bull. 2015; 41(2):419-428.
67. Ruhrmann S, Schultze-Lutter F, Schmidt S J, Kaiser N, Klosterkotter J. Prediction and prevention of psychosis: current progress and future tasks. Eur Arch Psychiatry Clin Neurosci. 2014; 264 Suppl 1:S9-16.
68. van der Gaag M, Smit F, Bechdolf A, French P, Linszen D H, Yung A R, McGorry P, Cuijpers P. Preventing a first episode of psychosis: meta-analysis of randomized controlled prevention trials of 12 month and longer-term follow-ups. Schizophr Res. 2013; 149(1-3):56-62.
69. Stafford M R, Jackson H, Mayo-Wilson E, Morrison A P, Kendall T. Early interventions to prevent psychosis: systematic review and meta-analysis. Bmj. 2013; 346:f185.
70. Hutton P, Taylor Pt Cognitive behavioural therapy for psychosis prevention: a systematic review and meta-analysis. Psychol Med. 2014; 44(3):449-468.
71. Preti A, Cella M. Randomized-controlled trials in people at ultra high risk of psychosis: a review of treatment effectiveness. Schizophr Res. 2010; 123(1):30-36.
72. Jaros J A, Martins-de-Souza D, Rahmoune H, Rothermundt M, Leweke F M, Guest P C, Bahn S. Protein phosphorylation patterns in serum from schizophrenia patients and healthy controls. Journal of proteomics. 2012; 76 Spec No.:43-55.
73. Yang Y, Wan C, Li H, Zhu H, La Y, Xi Z, Chen Y, Jiang L, Feng G, He L. Altered levels of acute phase proteins in the plasma of patients with schizophrenia. Anal Chem. 2006; 78(11):3571-3576.
74. Levin Y, Wang L, Schwarz E, Koethe D, Leweke F M, Bahn S. Global proteomic profiling reveals altered proteomic signature in schizophrenia serum. Mol Psychiatry. 2010; 15(11):1088-1100.
75. Hoirisch-Clapauch S, Amaral O B, Mezzasalma M A, Panizzutti R, Nardi A E. Dysfunction in the coagulation system and schizophrenia. Transl Psychiatry. 2016; 6:e704.
76. Kopczynska M, Zelek W, Touchard S, Gaughran F, Di Forti M, Mondelli V, Murray R, O'Donovan M C, Morgan B P. Complement system biomarkers in first episode psychosis. Schizophr Res. 2017; 17(pi is 50920-9964):30764-30768.
77. Boyajyan A, Khoyetsyan A, Chavushyan A. Alternative complement pathway in schizophrenia. Neurochem Res. 2010; 35(6):894-898.
78. Ricking M, Dicker P, Lopez L M, Cannon M, Schafer M R, McGorry P D, Smesny S, Cotter D R,
Amminger G P. Differential expression of the inflammation marker IL12p40 in the at-risk mental state for psychosis: a predictor of transition to psychotic disorder? BMC psychiatry. 2016; 16(1):326.
79. Miller B J, Buckley P, Seabolt W, Mellor A, Kirkpatrick B. Meta-analysis of cytokine alterations in schizophrenia: clinical status and antipsychotic effects. Biol Psychiatry. 2011; 70(7):663-671.
80. Khandaker G M, Pearson R M, Zammit S, Lewis G, Jones P B. Association of serum interleukin 6 and C-reactive protein in childhood with depression and psychosis in young adult life: a population-based longitudinal study. JAMA Psychiatry. 2014; 71(10):1121-1128.
81. van Beveren N J, Schwarz E, Noll R, Guest P C, Meijer C, de Haan L, Bahn S. Evidence for disturbed insulin and growth hormone signaling as potential risk factors in the development of schizophrenia. Transl Psychiatry. 2014; 4:e430.
82. Schwarz E, van Beveren N J, Ramsey J, Leweke F M, Rothermundt M, Bogerts B, Steiner J, Guest P C, Bahn S. Identification of subgroups of schizophrenia patients with changes in either immune or growth factor and hormonal pathways. Schizophr Bull. 2014; 40(4):787-795.
83. Baumeister D, Russell A, Pariante C M, Mondelli V. Inflammatory biomarker profiles of mental disorders and their relation to clinical, social and lifestyle factors. Soc Psychiatry Psychiatr Epidemiol. 2014; 49(6):841-849.
84. Laskaris L, Zalesky A, Weickert C S, Di Biase M A, Chana G, Baune B T, Bousman C, Nelson B, McGorry P, Everall I, Pantelis C, Cropley V. Investigation of peripheral complement factors across stages of psychosis. Schizophr Res. 2018.
85. Rehman A A, Ahsan H, Khan F H. alpha-2-Macroglobulin: a physiological guardian. J Cell Physiol. 2013; 228(8):1665-1675.
86. Upthegrove R, Manzanares-Teson N, Barnes N M. Cytokine function in medication-naive first episode psychosis: a systematic review and meta-analysis. Schizophr Res. 2014; 155(1-3):101-108.
87. Borth W. Alpha 2-macroglobulin, a multifunctional binding protein with targeting characteristics. Faseb j. 1992; 6(15):3345-3353.
88. de Boer J P, Creasey A A, Chang A, Abbink J J, Roem D, Eerenberg A J, Hack C E, Taylor F B, Jr. Alpha-2-macroglobulin functions as an inhibitor of fibrinolytic, clotting, and neutrophilic proteinases in sepsis: studies using a baboon model. Infect Immun. 1993; 61(12):5035-5043.
89. Baker S K, Chen Z L, Norris E H, Revenko A S, MacLeod A R, Strickland S. Blood-derived plasminogen drives brain inflammation and plaque deposition in a mouse model of Alzheimer's disease. Proc Natl Acad Sci USA. 2018; 115(41):E9687-E9696.
90. Amara U, Flierl M A, Rittirsch D, Klos A, Chen H, Acker B, Bruckner U B, Nilsson B, Gebhard F, Lambris J D, Huber-Lang M. Molecular intercommunication between the complement and coagulation systems. J Immunol. 2010; 185(9):5628-5636.
91. Ryu J K, Petersen M A, Murray S G, Baeten K M, Meyer-Franke A, Chan J P, Vagena E, Bedard C, Machado M R, Rios Coronado P E, Prod′homme T, Charo I F, Lassmann H, Degen J L, Zamvil S S, Akassoglou K. Blood coagulation protein fibrinogen promotes autoimmunity and demyelination via chemokine release and antigen presentation. Nat Commun. 2015; 6:8164.
92. Pollak T A, Drndarski S, Stone J M, David A S, McGuire P, Abbott N J. The blood-brain barrier in psychosis. Lancet Psychiatry. 2018; 5(1):79-92.
93. Ryu J K, Rafalski V A, Meyer-Franke A, Adams R A, Poda S B, Rios Coronado P E, Pedersen L O, Menon V, Baeten K M, Sikorski S L, Bedard C, Hanspers K, Bardehle S, Mendiola A S, Davalos D, Machado M R, Chan J P, Plastira I, Petersen M A, Pfaff S J, Ang K K, Hallenbeck K K, Syme C, Hakozaki H, Ellisman M H, Swanson R A, Zamvil S S, Arkin M R, Zorn S H, Pico A R, Mucke L, Freedman S B, Stavenhagen J B, Nelson R B, Akassoglou K. Fibrin-targeting immunotherapy protects against neuroinflammation and neurodegeneration. Nat Immunol. 2018; 19(11):1212-1223.
94. Comes A L, Papiol S, Mueller T, Geyer P E, Mann M, Schulze T G. Proteomics for blood biomarker exploration of severe mental illness: pitfalls of the past and potential for the future. Translational Psychiatry. 2018; 8(1):160.
95. Deighton S, Neville A, Pusch D, Dobson K. Biomarkers of adverse childhood experiences: A scoping review. Psychiatry Res. 2018; 269:719-732.
96. Baumeister D, Akhtar R, Ciufolini S, Pariante C M, Mondelli V. Childhood trauma and adulthood inflammation: a meta-analysis of peripheral C-reactive protein, interleukin-6 and tumour necrosis factor-α. Molecular psychiatry. 2016; 21(5):642-649.
97. Rasmussen U H, Moffitt T E, Arseneault L, Danese A, Eugen-Olsen J, Fisher H L, Harrington H, Houts R, Matthews T, Sugden K, Williams B, Caspi A. Association of Adverse Experiences and Exposure to Violence in Childhood and Adolescence With Inflammatory Burden in Young People. JAMA Pediatrics. 2019:1-11.

### Supplementary Material & Methods

### Sample Preparation

### Blood Collection

Plasma P100 tubes were used for blood collection and samples were stored on ice for a maximum of 90 minutes until processed, centrifuged and stored in a −80° C. freezer. The standard quality of the plasma samples was ensured by assessing the overall MS protein profile to facilitate the identification of outlier protein expression profiles.

### Protein Depletion of Plasma Samples

To improve the dynamic range for proteomic analysis, 40 μl of plasma from each case in all samples was immunodepleted of the 14 most abundant proteins (α-1-antitrypsin, A1-acid glycoprotein, Serum Albumin, α2-macroglobulin, Apolipoprotein A-I, Apolipoptrotein A-II, Complement C3, Fibrinogen α/β/γ, Haptoglobin, IgA, IgG, IgM, Transthyretin, and Serotransferrin), using the Agilent Hu14 Affinity Removal System (MARS) coupled to a High Performance Liquid Chromatography (HPLC) system¹. Protein depletion was undertaken according to the manufacturer's instructions and buffer exchange was performed with 50 mM ammonium bicarbonate using spin columns with a 10 kDA-molecular weight cut-off (Merck Millipore). Prior to sample preparation for mass spectrometry (MS), the protein concentration was determined using a Bradford Assay², according to the manufacturer's (BioRad) instructions.

### Sample Preparation for Mass Spectrometry

Protein digestion and peptide purification was performed as previously described³. For quality control (QC), an equal aliquot from each protein digest in the experiment was pooled into one sample for use as an internal QC. This QC standard was injected at the beginning of the MS study to condition the column, and after every ten injections throughout the experiment to monitor the MS performance.

### Discovery Proteomic Analysis Using Data Dependent Acquisition (DDA)

All samples were injected on a Thermo Scientific Q Exactive mass spectrometer connected to a Dionex Ultimate 3000 (RSLCnano) chromatography system. Tryptic peptides (5 μl of digest) from each sample were loaded onto a fused silica emitter (75 μm ID, pulled using a laser puller (Sutter Instruments P2000), packed with UChrom C18 (1.8 μm) reverse phase media (nanoLCMS Solutions LCC) and was separated by an increasing acetonitrile gradient over 90 minutes at a flow rate of 250 nL/min. This QC standard was injected 3 times at the beginning of the MS study to condition the column, and after every ten injections throughout the experiment to monitor the MS performance. The mass spectrometer was operated in data dependent TopN 8 mode, with the following settings: mass range 300-1600 Th; resolution for MS1 scan 70000; A Vitamin D binding protein target 3e6; resolution for MS2 scan 17500; A Vitamin D binding protein target 2e4; charge exclusion unassigned, 1; dynamic exclusion 40 s.

### Confirmatory Analyses by ELISA

To validate our findings, we assessed several human complement and coagulation proteins and apolipoproteins in the plasma samples of the same CHR-T and CHR-NT subjects who contributed to the proteomic study using enzyme-linked immunoassays (ELISA). Candidate proteins were chosen on the basis of machine learning results and differential expression as well as previous study results from our group (⁴⁻⁷). Specifically, we tested human α-2 macroglobulin (Abcam ab108888, 1:400), apolipoprotein E (ThermoFisher Scientific, EHAPOE, 1:2,000), Complement C1q (Abcam ab170246, 1:100,000), Complement C1r (Abcam ab170245, 1:40,000), Complement C4 binding protein (Abcam, ab222866, 1:40,000), Complement C8 (Abcam ab 137971, 1:10,000), complement factor H (Hykult Biotech HK342, 1:10,000), immunoglobulin M (Abcam, ab137982, 1:60,000), and plasminogen (Abcam ab108893, 1:20,000) in accordance with the manufacturer's instructions. Concentrations for unknown samples were interpolated using 4 parameter logistic curve fit in GraphPad Prism 8 software and means for each protein were compared using the t-test with unequal variances in Stata version 15.

### Bioinformatics and Statistical Analysis

### EU-GEI Clinical Variables

A full list of the baseline clinical variables included is provided in eTable 1.

### Leave-Site-Out Cross-Validation

The data were first split into a number of folds in the ‘outer loop’ of cross-validation. To incorporate geographical generalisability, we split the data by study site. Several of the smaller sites from the EU-GEI study were combined to ensure a large enough transition sample was present at each site. Thus, Amsterdam and The Hague were combined, Vienna and Basel were combined, Copenhagen and Paris were combined, and Barcelona and Sao Paulo were combined. This resulted in 6 final sites which were folds in the outer cross-validation loop (% transition): London (35.2%), the Netherlands (12.5%), Switzerland/Austria (57.1%), Melbourne (35.7%), Denmark/France (45.8%) and Spain/Brazil (30.8%). For each cycle of cross-validation, data from each of the 6 sites were held out and the rest of the data moved into the ‘inner loop’ for training.

### Repeated Nested Cross-Validation

Within the inner loop, we used 5 non-overlapping folds with iterative training-test cycles. Thus, training was applied to four-fifths of the data in the inner loop and then tested against the final one-fifth, with the five different inner loop folds as the test fold. Models were trained and tested within the inner loop using a range of regularisation parameter values (in 11 steps from 0.015625 to 16).

The optimal models thus derived were tested against the held-out site in the outer loop. This process was then repeated, with each site in the outer loop as the test site, to determine the overall optimal model and final predictive accuracy. For a detailed description of repeated nested cross-validation, see the Supplementary material of Koutsouleris et al⁸ and the Neurominer manual (available from https//www pronia.eu/neurominer/).

### Confidence Intervals for Area Under the Curve

95% confidence intervals for the area under the receiver-operating curve (AUC) for each model were calculated according to the method of Hanley & McNeil.⁹

### EU-GEI Replication Dataset

The replication dataset included 49 CHR-T participants (2 of whom were different from those in Models 1a-d) and 86 CHR-NT participants (all of whom were different from those in Models 1a-d). Characteristics of participants included in the replication dataset are compared to those not included in Table 9. Included participants were more likely to be male, but otherwise were comparable with non-included participants on baseline characteristics. Characteristics for CHR-T and CHR-NT participants included in the replication dataset are compared in Table 10. There was evidence of differences for ethnicity and tobacco use between the two groups, higher mean total SANS global and composite scores and total BPRS score in CHR-T.

### Supplementary References

1. Levin Y, Wang L, Schwarz E, Koethe D, Leweke F M, Balm S. Global proteomic profiling reveals altered proteomic signature in schizophrenia serum. Mol Psychiatry. 2010; 15(11):1088-1100.
2. Bradford M M. A rapid and sensitive method for the quantitation of microgram quantities of protein utilizing the principle of protein-dye binding. Anal Biochem. 1976; 72:248-254.
3. English J A, Fan Y, Focking M, Lopez L M, Hryniewiecka M, Wynne K, Dicker P, Matigian N, Cagney G, Mackay-Sim A, Cotter D R. Reduced protein synthesis in schizophrenia patient-derived olfactory cells. Transl Psychiatry. 2015; 5:e663.
4. English J A, Lopez L M, O'Gorman A, Focking M, Hryniewiecka M, Scaife C, Sabherwal S, Wynne K, Dicker P, Rutten B P F, Lewis G, Zammit S, Cannon M, Cagney G, Cotter D R. Blood-Based Protein Changes in Childhood Are Associated With Increased Risk for Later Psychotic Disorder: Evidence From a Nested Case-Control Study of the ALSPAC Longitudinal Birth Cohort. Schizophr Bull. 2018; 44(2):297-306.
5. Ricking M, Sabherwal S, Cates H M, Scaife C, Dicker P, Hryniewiecka M, Wynne K, Rutten B P F, Lewis G, Cannon M, Nestler E J, Heurich M, Cagney G, Zammit S, Cotter D R. Complement pathway changes at age 12 are associated with psychotic experiences at age 18 in a longitudinal population-based study: evidence for a role of stress. Molecular Psychiatry. 2019.
6. Sabherwal S, English J A, Focking M, Cagney G, Cotter D R. Blood biomarker discovery in drug-five schizophrenia: the contribution of proteomics and multiplex immunoassays. Expert Rev Proteomics. 2016; 13(12):1141-1155.
7. Sabherwal S, Focking M, English J A, Fitzsimons S, Hryniewiecka M, Wynne K, Scaife C, Healy C, Cannon M, Belton O, Zammit S, Cagney G, Cotter D R. ApoE elevation is associated with the persistence of psychotic experiences from age 12 to age 18: Evidence from the ALSPAC birth cohort. Schizophr Res. 2019.
8. Koutsouleris N, Kahn R S, Chekroud A M, Leucht S, Falkai P, Wobrock T, Derks E M, Fleischhacker W W, Hasan A. Multisite prediction of 4-week and 52-week treatment outcomes in patients with first-episode psychosis: a machine learning approach Lancet Psychiatry. 2016; 3(10):935-946.
9. Hanley J A, McNeil B J. The meaning and use of the are under a receiver operating characteristic (ROC) curve. Radiology. 1982 April; 143(1):29-36.

TABLE 4
List of 65 baseline clinical variables
included in EU-GEI support vector models

GAF symptoms
GAF disability
SANS: unchanging facial expression
SANS: decreased spontaneous movements
SANS: paucity of expressive gestures
SANS: poor eye contact
SANS: affective nonresponsivity
SANS: inappropriate affect
SANS: lack of vocal inflections
SANS: global rating of affective flattening
SANS: poverty of speech
SANS: poverty of speech content
SANS: blocking
SANS: increased latency of response
SANS: global rating of alogia
SANS: grooming and hygiene
SANS: impersistence at work or school
SANS: physical anergia
SANS: global rating for avolition-apathy
SANS: recreational interests and activities
SANS: sexual activity
SANS: ability to feel intimacy and closeness
SANS: relationship with friends and peers
SANS: global rating of anhedonia-asociality
SANS: social inattentiveness
SANS: inattentiveness during mental status testing
SANS: global rating of attention
Total SANS composite score
Total SANS global score
BPRS: somatic concern
BPRS: anxiety
BPRS: depression
BPRS: suicidality
BPRS: guilt
BPRS: hostility
BPRS: elevated mood
BPRS: grandiosity
BORS: suspiciousness
BPRS: hallucinations
BPRS: unusual thought content
BPRS: bizarre behaviour
BPRS: self-neglect
BPRS: disorientation
BPRS: conceptual disorganisation
BPRS: blunted affect
BPRS: emotional withdrawal
BPRS: motor retardation
BPRS: tension
BPRS: uncooperativeness
BPRS: excitement
BPRS: distractibility
BPRS: motor hyperactivity
BPRS: mannerisms and posturing
Total BPRS score
MADRS: apparent sadness
MADRS: reported sadness
MADRS: inner tension
MADRS: reduced sleep
MADRS: reduced appetite
MADRS: concentration difficulties
MADRS: lassitude
MADRS: inability to feel
MADRS: pessimistic thoughts
MADRS: suicidal thoughts
Total MADRS score
GAF: General Assessment of Functioning; SANS: Scale for the Assessment of Negative Symptoms;
BPRS: Brief Psychiatric Rating Scale; MADRS: Montgomery Asberg Depression Rating Scale

TABLE 5
Comparison of characteristics for participants included in original experiment
(N = 133) from total EU-GEI CHR cohort (N = 344)

| | | | Included, | | Not included, | | | |
|---|---|---|---|---|---|---|---|---|
| | | | N = 133 | | N = 211 | | | |
| | Missing | | (49 CHR-T, | | (16 CHR-T, | | | |
| | data, n (%) | | 84 CHR-NT) | | 195 CHR-NT) | | t/χ² | p |
| Baseline age | 0 | | 22.6 | (4.5) | 22.3 | (5.2) | 0.686 | 0.493 |
| in years, | | | | | | | | |
| mean (SD) | | | | | | | | |
| Sex, n (%) | 0 | | 68 male | (51.1%) | 117 male | (55.5%) | 0.613 | 0.434 |
| | | | 65 female | (49.9%) | 94 female | (45.5%) | | |
| Baseline | 50 | (14.5%) | 24.4 | (5.6) | 23.7 | (4.9) | 1.190 | 0.235 |
| BMI in | | | | | | | | |
| kg/m², | | | | | | | | |
| mean (SD) | | | | | | | | |
| Baseline | 38 | (11.0%) | 14.3 | (3.1) | 14.4 | (3.0) | −0.318 | 0.751 |
| years in | | | | | | | | |
| education, | | | | | | | | |
| mean (SD) | | | | | | | | |
| Ethnicity, n | 0 | | 91 white | (68.4%) | 156 white | (73.9%) | 1.239 | 0.538 |
| (%) | | | 15 black | (11.3%) | 19 black | (9.0%) | | |
| | | | 27 other | (20.3%) | 36 other | (17.1%) | | |
| Ever used | 10 | (2.9%) | 101 yes | (75.9%) | 143 yes | (67.8%) | 2.326 | 0.127 |
| cannabis, | | | 29 no | (21.8%) | 61 no | (28.9%) | | |
| n (%) | | | 3 not known | (2.3%) | 7 not known | (3.3%) | | |
| Baseline | 95 | (27.6%) | 41 yes | (30.8%) | 47 yes | (22.3%) | 1.302 | 0.254 |
| cannabis | | | 63 no | (47.4%) | 98 no | (46.4%) | | |
| use, n (%) | | | 29 not known | (21.8%) | 66 not known | (31.3%) | | |
| Baseline | 38 | (11.0%) | 64 yes | (48.1%) | 97 yes | (46.0%) | 0.106 | 0.744 |
| tobacco use, | | | 55 no | (41.4%) | 90 no | (42.7%) | | |
| n (%) | | | 14 not known | (10.5%) | 24 not known | (11.4%) | | |
| Baseline | 12 | (3.5%) | 93 yes | (69.9%) | 141 yes | (66.8%) | 0.115 | 0.735 |
| alcohol use, | | | 37 no | (27.8%) | 61 no | (28.9%) | | |
| n (%) | | | 3 not known | (2.3%) | 9 not known | (4.3%) | | |
| Baseline | 86 | (25.0%) | 51 yes | (38.3%) | 78 yes | (37.0%) | 0 | 1.0 |
| medication | | | Antidepressant | 29 | Antidepressant | 45 | | |
| use, n (%) | | | Antipsychotic | 9 | Antipsychotic | 11 | | |
| | | | Hypnotic | 7 | Hypnotic | 7 | | |
| | | | Other | 6 | Other | 15 | | |
| | | | 51 no | (38.3%) | 78 no | (37.0%) | | |
| | | | 31 not known | (23.3%) | 55 not known | (26.1%) | | |
| Baseline | 27 | (7.8%) | 54.7 | (10.2) | 55.4 | (10.0) | −0.634 | 0.526 |
| GAF | | | | | | | | |
| symptoms | | | | | | | | |
| score, mean | | | | | | | | |
| (SD) | | | | | | | | |
| Baseline | 12 | (3.5%) | 53.9 | (11.7) | 56.4 | (12.5) | −1.827 | 0.069 |
| GAF | | | | | | | | |
| disability | | | | | | | | |
| score, mean | | | | | | | | |
| (SD) | | | | | | | | |
| Baseline | 45 | (13.1%) | 18.0 | (12.7) | 14.2 | (10.7) | 2.782 | 0.006 |
| SANS total | | | | | | | | |
| composite | | | | | | | | |
| score, mean | | | | | | | | |
| (SD) | | | | | | | | |
| Baseline | 29 | (8.4%) | 6.1 | (3.9) | 5.0 | (3.4) | 2.528 | 0.012 |
| SANS total | | | | | | | | |
| global score, | | | | | | | | |
| mean (SD) | | | | | | | | |
| Baseline | 25 | (7.3%) | 46.0 | (10.9) | 42.2 | (9.6) | 3.174 | 0.002 |
| BPRS total | | | | | | | | |
| score, mean | | | | | | | | |
| (SD) | | | | | | | | |
| Baseline | 16 | (4.7%) | 19.6 | (9.7) | 18.4 | (8.8) | 1.155 | 0.249 |
| MADRS | | | | | | | | |
| total score, | | | | | | | | |
| mean (SD) | | | | | | | | |
| 2 year GAF | 142 | (41.3%) | 54.6 | (15.0) | 63.0 | (11.6) | −4.083 | <0.001 |
| symptoms | | | | | | | | |
| score, mean | | | | | | | | |
| (SD) | | | | | | | | |
| 2 year GAF | 124 | (36.0%) | 56.9 | (15.0) | 63.6 | (13.8) | −3.333 | 0.001 |
| disability | | | | | | | | |
| score, mean | | | | | | | | |
| (SD) | | | | | | | | |
| 2 year GAF | 124 | (36.0%) | 32 good | (24.1%) | 80 good | (37.9%) | 5.337 | 0.021 |

| | | | | |
|---|---|---|---|---|
| disability | 47 poor | (35.3%) | 61 poor | (28.9%) |
| score, | 54 not known | (40.6%) | 70 not known | (33.2%) |

dichotomous
outcome ^{a}

^{a} Poor functioning: GAF disability score ≤60; good functioning: GAF disability score >60
Tobacco use was defined as daily use for at least 1 month over the previous 12 months. Alcohol use was defined as at least 12 or more alcoholic beverages over the previous 12 months. Missing data excluded in hypothesis tests.
EU-GEI: European Union Gene Environment Interaction study; CHR-T: clinical high risk, transitioned to psychosis; CHR-NT: clinical high risk, did not transition to psychosis; BMI: body mass index; GAF: General Assessment of Functioning; SANS: Scale for the Assessment of Negative Symptoms; BPRS: Brief Psychiatric Rating Scale; MADRS: Montgomery Asberg Depression Rating Scale

TABLE 6
Results of ANCOVA and fold changes (CHR-T vs. CHR-NT) for proteins identified in EU-
GEI baseline plasma samples (adjusted for age, sex, BMI and years in education)

| | | | | | | Mean LFQ, | Ratio of |
|---|---|---|---|---|---|---|---|
| | | | | | Mean LFQ, | non- | means |
| | | | | FDR | transition | transition | (T vs |
| Uniprot | Name | F | p | 5% | group | group | NT] |
| P01023 | Alpha-2-macroglobulin | 146 | 7.55E−23 | <0.001 | 1.14E+09 | 3.49E+09 | 0.33 |
| P01871 | Immunoglobulin heavy | 72.16 | 4.53E−14 | <0.001 | 1.1E+08 | 2.71E+08 | 0.41 |
| | constant mu | | | | | | |
| P07357 | Complement component C8 | 44.25 | 7.76E−10 | <0.001 | 2.5E+08 | 1.69E+08 | 1.48 |
| | alpha chain | | | | | | |
| P02774 | Vitamin D-binding protein | 40.97 | 2.72E−09 | <0.001 | 7.4E+09 | 5.17E+09 | 1.43 |
| P02747 | Complement C1q | 36.52 | 1.56E−08 | <0.001 | 2.06E+08 | 1.35E+08 | 1.53 |
| | subcomponent subunit C | | | | | | |
| P00747 | Plasminogen | 31.39 | 1.25E−07 | <0.001 | 4.09E+09 | 3.17E+09 | 1.29 |
| P10909 | Clusterin | 29.74 | 2.48E−07 | <0.001 | 1.64E+09 | 1.27E+09 | 1.29 |
| P23142 | Fibulin-1 | 28.56 | 4.06E−07 | <0.001 | 83608657 | 55104835 | 1.52 |
| P55058 | Phospholipid transfer protein | 19.09 | 2.57E−05 | <0.001 | 7342621 | 10962754 | 0.67 |
| P00736 | Complement C1r | 18.07 | 4.09E−05 | <0.001 | 9.9E+08 | 7.81E+08 | 1.27 |
| | subcomponent | | | | | | |
| P08603 | Complement factor H | 16.18 | 0.0001 | 0.001 | 4.43E+09 | 3.81E+09 | 1.16 |
| P05156 | Complement factor I | 16.72 | 0.0001 | 0.001 | 1.42E+09 | 1.16E+09 | 1.23 |
| O75882 | Attractin | 16.85 | 0.0001 | 0.001 | 1.17E+08 | 90521420 | 1.30 |
| P03951 | Coagulation factor XI | 17.61 | 0.0001 | 0.001 | 44890311 | 32936650 | 1.36 |
| P43320 | Beta-crystallin B2 | 16.39 | 0.0001 | 0.001 | 1.23E+08 | 63601609 | 1.80 |
| P04003 | C4b-binding protein alpha | 14.71 | 0.0002 | 0.002 | 6.96E+08 | 9.15E+08 | 0.76 |
| | chain | | | | | | |
| P19827 | Inter-alpha-trypsin inhibitor | 15.05 | 0.0002 | 0.002 | 2.95E+09 | 2.49E+09 | 1.19 |
| | heavy chain H1 | | | | | | |
| O75636 | Ficolin-3 | 14.09 | 0.0003 | 0.003 | 1.48E+08 | 2.11E+08 | 0.70 |
| P01860 | Immunoglobulin heavy | 13.19 | 0.0004 | 0.003 | 5.61E+08 | 8.04E+08 | 0.70 |
| | constant gamma 3 | | | | | | |
| P15144 | Aminopeptidase N | 13.38 | 0.0004 | 0.003 | 10232560 | 14138254 | 0.72 |
| P02489 | Alpha-crystallin A chain | 12.08 | 0.0007 | 0.006 | 1.81E+08 | 1.11E+08 | 1.63 |
| P06396 | Gelsolin | 11.83 | 0.0008 | 0.006 | 1.92E+09 | 1.64E+09 | 1.17 |
| Q14520 | Hyaluronan-binding protein 2 | 11.87 | 0.0008 | 0.006 | 1.31E+08 | 1.08E+08 | 1.21 |
| P05155 | Plasma pretease C1 inhibitor | 11.01 | 0.0012 | 0.008 | 1.66E+09 | 1.95E+09 | 0.85 |
| P02766 | Transthyretin | 10.79 | 0.0013 | 0.009 | 21337361 | 35624572 | 0.60 |
| P04217 | Alpha-1B-glycoprotein | 9.97 | 0.002 | 0.012 | 3.37E+09 | 4.04E+09 | 0.83 |
| P02749 | Beta-2-glycoprotein 1 | 9.93 | 0.002 | 0.012 | 5.76E+09 | 4.54E+09 | 1.27 |
| P22891 | Vitamin K-dependert protein Z | 9.86 | 0.0021 | 0.012 | 24836850 | 36479890 | 0.68 |
| P00751 | Complement factor B | 9.41 | 0.0026 | 0.015 | 4.72E+09 | 4.04E+09 | 1.17 |
| P05546 | Heparin cofactor 2 | 8.91 | 0.0034 | 0.019 | 2.04E+09 | 2.43E+09 | 0.84 |
| P06276 | Cholinesterase | 8.71 | 0.0038 | 0.020 | 1.5E+08 | 1.26E+08 | 1.18 |
| P51884 | Lumican | 8.4 | 0.0044 | 0.023 | 5.93E+08 | 4.74E+08 | 1.25 |
| P02649 | Apolipoprotein E | 8.25 | 0.0048 | 0.024 | 9.1E+08 | 7.1E+08 | 1.28 |
| Q76LX8 | A disintegrin and | 7.56 | 0.0068 | 0.033 | 9713735 | 11499010 | 0.84 |
| | metalloproteinase with | | | | | | |
| | thrombospondin motifs 13 | | | | | | |
| Q06033 | Inter-alpha-trypsin inhibitor | 7.15 | 0.0085 | 0.040 | 2.04E+08 | 2.75E+08 | 0.74 |
| | heavy chain H3 | | | | | | |
| P02656 | Apolipoprotein C-III | 6.59 | 0.0114 | 0.053 | 4.4E+08 | 3.22E+08 | 1.37 |
| P05543 | Thyroxine-binding globulin | 6.43 | 0.0124 | 0.054 | 1.93E+08 | 1.64E+08 | 1.18 |
| P02751 | Fibronectin | 6.43 | 0.0124 | 0.054 | 7.41E+09 | 5.71E+09 | 1.30 |
| P00450 | Ceruloplasmin | 6.32 | 0.0132 | 0.056 | 5.67E+09 | 4.99E+09 | 1.14 |
| Q04756 | Hepatocyte growth factor | 5.69 | 0.0185 | 0.077 | 1.08E+38 | 94519792 | 1.14 |
| | activator | | | | | | |
| P05090 | Apolipoprotein D | 5.61 | 0.0193 | 0.078 | 32446284 | 27798720 | 1.17 |
| Q08380 | Galectin-3-binding protein | 5.38 | 0.0219 | 0.087 | 9803809 | 12738574 | 0.77 |
| P11226 | Mannose-binding protein C | 5.18 | 0.0246 | 0.092 | 56219776 | 41212566 | 1.36 |
| P10643 | Complement component C7 | 5.17 | 0.0247 | 0.092 | 5.43E+08 | 4.64E+08 | 1.17 |
| P07225 | Vitamin K-dependent protein S | 5.12 | 0.0254 | 0.092 | 2.3E+08 | 2.64E+08 | 0.87 |
| Q9BXR6 | Complement factor H-related | 5.1 | 0.0256 | 0.092 | 30517136 | 23408385 | 1.30 |
| | protein 5 | | | | | | |
| P49747 | Cartilage oligomeric matrix | 4.94 | 0.028 | 0.099 | 21691930 | 18636716 | 1.16 |
| | protein | | | | | | |
| P02675 | Fibrinogen beta chain | 4.79 | 0.0305 | 0.103 | 6.19E+08 | 5.39E+08 | 1.15 |
| P02671 | Fibrinogen alpha chain | 4.77 | 0.0307 | 0.103 | 5.85E+08 | 5.24E+08 | 1.12 |
| P04114 | Apolipoprotein B-100 | 4.75 | 0.0311 | 0.103 | 1.92E+10 | 2.16E+10 | 0.89 |
| P01042 | Kininogen-1 | 4.69 | 0.0322 | 0.105 | 4.3E+09 | 3.94E+09 | 1.09 |
| P43652 | Afamin | 4.49 | 0.0361 | 0.115 | 1.92E+09 | 1.72E+09 | 1.12 |
| P05160 | Coagulation factor XIII B chain | 4.25 | 0.041 | 0.125 | 1.05E+08 | 91990562 | 1.14 |
| Q9NZP8 | Complement C1r | 4.23 | 0.0417 | 0.125 | 79864565 | 62745563 | 1.27 |
| | subcomponent-like protein | | | | | | |
| P02753 | Retinol-binding protein 4 | 4.22 | 0.0421 | 0.125 | 7.38E+08 | 5.44E+08 | 1.36 |
| P00742 | Coagulation factor X | 4.21 | 0.0423 | 0.125 | 1.66E+08 | 1.43E+08 | 1.11 |
| P36980 | Complement factor H-related | 3.72 | 0.0559 | 0.163 | 84913609 | 65032425 | 1.31 |
| | protein 2 | | | | | | |
| P07195 | L-lactate dehydrogenase B | 3.68 | 0.0572 | 0.164 | 29579331 | 25550208 | 2.16 |
| | chain | | | | | | |
| P02748 | Complement component C9 | 3.64 | 0.0585 | 0.165 | 1.3E+09 | 1.43E+09 | 0.91 |
| P01024 | Complement C3 | 3.56 | 0.0615 | 0.170 | 4.52E+08 | 3.99E+08 | 1.13 |
| P01876 | Immunoglobulin heavy | 3.4 | 0.0675 | 0.184 | 1.84E+08 | 1.62E+08 | 1.14 |
| | constant alpha 1 | | | | | | |
| P02743 | Serum amyloid P-component | 3.33 | 0.0705 | 0.189 | 8.62E+08 | 7.7E+08 | 2.22 |
| P05452 | Tetranectin | 3.21 | 0.0756 | 0.199 | 5.07E+08 | 4.55E+08 | 1.21 |
| P20742 | Pregnancy zone protein | 2.87 | 0.093 | 0.241 | 85801269 | 1.13E+08 | 0.76 |
| P00740 | Coagulation factor IX | 2.83 | 0.0949 | 0.242 | 1.58E+08 | 1.41E+08 | 1.12 |
| P01859 | Immunoglobulin heavy | 2.55 | 0.1125 | 0.283 | 73571215 | 65265721 | 1.13 |
| | constant gamma 2 | | | | | | |
| P00748 | Coagulation factor XII | 2.49 | 0.117 | 0.290 | 6.19E+08 | 5.44E+06 | 1.14 |
| P68871 | Hemoglobin subunit beta | 2.3 | 0.1315 | 0.317 | 1.92E+08 | 1.45E+08 | 1.33 |
| P02787 | Serotransferrin | 2.3 | 0.1316 | 0.317 | 7.38E+08 | 7.06E+08 | 1.05 |
| P01011 | Alpha-1-antichymotrypsin | 2.27 | 0.1347 | 0.319 | 5.65E+09 | 5.31E+09 | 1.06 |
| P07737 | Profilin-1 | 2.07 | 0.1526 | 0.357 | 42263756 | 56358056 | 0.75 |
| Q92954 | Proteoglycan 4 | 2.03 | 0.1565 | 0.361 | 47223124 | 53868662 | 0.88 |
| P13671 | Complement component C6 | 1.86 | 0.1745 | 0.397 | 8.74E+08 | 8.26E+08 | 1.06 |
| P09871 | Complement C1s | 1.84 | 0.1776 | 0.398 | 7.8E+08 | 7.36E+08 | 1.06 |
| | subcomponent | | | | | | |
| P07358 | Complement component C8 | 1.77 | 0.1856 | 0.405 | 3.71E+08 | 4.06E+08 | 0.91 |
| | beta chain | | | | | | |
| Q03591 | Complement factor H-related | 1.77 | 0.1856 | 0.405 | 81709939 | 68757368 | 1.19 |
| | protein 1 | | | | | | |
| P02654 | Apolipoprotein C-I | 1.55 | 0.2147 | 0.463 | 1.31E+08 | 1.9E+08 | 0.69 |
| P48740 | Mannan-binding lectin serine | 1.43 | 0.2336 | 0.497 | 35332742 | 32142164 | 1.10 |
| | protease 1 | | | | | | |
| P02760 | Protein AMBP [Cleaved into: | 1.41 | 0.2371 | 0.498 | 1.29E+09 | 1.19E+09 | 1.08 |
| | Alpha-1-microglobulin | | | | | | |
| P02790 | Hemopexin | 1.37 | 0.2439 | 0.503 | 9.58E+09 | 1.02E+10 | 0.94 |
| P29622 | Kallistatin | 1.34 | 0.2484 | 0.503 | 6.97E+08 | 6.58E+08 | 1.06 |
| P04004 | Vitronectin | 1.34 | 0.2486 | 0.503 | 3.24E+09 | 3.38E+09 | 0.96 |
| P14618 | Pyruvate kinase PKM | 1.32 | 0.253 | 0.506 | 21521028 | 25635061 | 0.84 |
| P22792 | Carboxypeptidase N subunit 2 | 1.26 | 0.2639 | 0.522 | 2.59E+08 | 2.44E+08 | 1.06 |
| P17936 | Insulin-like growth factor- | 1.21 | 0.2727 | 0.527 | 1.37E+08 | 1.5E+08 | 0.91 |
| | binding protein 3 | | | | | | |
| Q96PD5 | N-acetylmuramoyl-L-alanine | 1.21 | 0.2734 | 0.527 | 5.23E+08 | 5.63E+08 | 0.93 |
| | amidase | | | | | | |
| P01019 | Angiotensinogen | 1.19 | 0.2767 | 0.527 | 3.35E+09 | 3.75E+09 | 0.89 |
| Q96XN2 | Beta-Ala-His dipeptidase | 1.17 | 0.2808 | 0.527 | 93683454 | 1.02E+08 | 0.92 |
| P02652 | Apolipoprotein A-II | 1.15 | 0.2828 | 0.527 | 3.59E+08 | 3.7E+08 | 0.97 |
| P27169 | Serum | 1.13 | 0.2894 | 0.534 | 72098714 | 65029166 | 1.21 |
| | paraoxonase/arylesterase 1 | | | | | | |
| P22105 | Tenascin-X | 1.08 | 0.3001 | 0.547 | 31438477 | 27910277 | 1.13 |
| P09172 | Dopamine beta-hydroxylase | 0.95 | 0.3314 | 0.598 | 28931848 | 26122837 | 1.11 |
| P60174 | Triosephosphate isomerase | 0.89 | 0.3481 | 0.621 | 8691317 | 10128185 | 0.86 |
| P15169 | Carboxypeptidase N catalytic | 0.8 | 0.3724 | 0.641 | 1.58E+08 | 1.48E+08 | 1.07 |
| | chain | | | | | | |
| P08185 | Corticosteroid-binding globulin | 0.8 | 0.3725 | 0.641 | 2E+08 | 2.3E+08 | 0.87 |
| P23528 | Cofilin-1 | 0.8 | 0.3737 | 0.641 | 37675779 | 33910502 | 1.11 |
| P80108 | Phosphatidylinositol-glycan- | 0.79 | 0.3746 | 0.641 | 2.47E+08 | 2.28E+08 | 1.08 |
| | specific phospholipase D | | | | | | |
| P02775 | Platelet basic protein | 0.78 | 0.3801 | 0.644 | 1.93E+08 | 2.47E+08 | 0.78 |
| P06681 | Complement C2 | 0.72 | 0.3974 | 0.666 | 2.7E+08 | 2.87E+08 | 0.94 |
| P00734 | Prothrombin | 0.67 | 0.413 | 0.674 | 4.54E+09 | 4.41E+09 | 1.03 |
| P60709 | Actin, cytoplasmic 1 | 0.67 | 0.4157 | 0.674 | 3.01E+08 | 2.93E+08 | 1.03 |
| P35858 | Insulin-like growth factor- | 0.66 | 0.4173 | 0.674 | 6.57E+08 | 6.95E+08 | 0.94 |
| | binding protein complex acid | | | | | | |
| | labile subunit | | | | | | |
| P02647 | Apolipoprotein A-I | 0.66 | 0.4185 | 0.674 | 8.83E+08 | 9.95E+08 | 0.89 |
| P02763 | Alpha-1-acid glycoprotein 1 | 0.63 | 0.4285 | 0.677 | 1.61E+08 | 1.63E+08 | 0.99 |
| P04075 | Fructose-bisphosphate | 0.62 | 0.4332 | 0.677 | 39556865 | 41635811 | 0.95 |
| | aldolase A | | | | | | |
| P18428 | Lipopolysaccharide-binding | 0.62 | 0.4335 | 0.677 | 1.3E+08 | 1.43E+08 | 0.91 |
| | protein | | | | | | |
| P01834 | Immunuglobulin kappa | 0.61 | 0.4363 | 0.677 | 1.28E+08 | 1.31E+08 | 0.98 |
| | constant | | | | | | |
| P04196 | Histidine-rich glycoprotein | 0.58 | 0.4474 | 0.688 | 1.51E+09 | 1.4E+09 | 1.08 |
| Q9V6R7 | IgGFc-binding protein | 0.56 | 0.456 | 0.691 | 45729367 | 44506955 | 1.03 |
| P08697 | Alpha-2-antiplasmin | 0.55 | 0.4594 | 0.691 | 1.13E+09 | 1.16E+09 | 0.97 |
| Q14624 | Inter-alpha-trypsin inhibitor | 0.54 | 0.4618 | 0.691 | 4.43E+09 | 4.35E+09 | 1.02 |
| | heavy chain H4 | | | | | | |
| P00738 | Haptoglobin | 0.53 | 0.468 | 0.694 | 4.45E+08 | 7.47E+08 | 0.60 |
| O43866 | CD5 antigen-like | 0.5 | 0.4793 | 0.698 | 39577655 | 42728179 | 0.93 |
| P0C0L5 | Complement C4-B | 0.5 | 0.4794 | 0.698 | 3.45E+08 | 3.07E+08 | 1.12 |
| Q12913 | Receptor-type tyrosine-protein | 0.49 | 0.4863 | 0.702 | 8940384 | 8582967 | 1.04 |
| | phosphatase eta | | | | | | |
| P04070 | Vitamin K-dependent protein C | 0.44 | 0.5103 | 0.730 | 41642694 | 47336839 | 0.88 |
| P12814 | Alpha-actinin-1 | 0.41 | 0.5251 | 0.739 | 33166338 | 40502857 | 0.82 |
| P11021 | Endoplasmic reticulum chaperone BiP | 0.41 | 0.5252 | 0.739 | 10424759 | 10245287 | 1.02 |
| P04275 | von Willebrand factor | 0.36 | 0.5505 | 0.768 | 96700211 | 89150500 | 1.08 |
| P36955 | Pigment epithelium-derived factor | 0.33 | 0.5696 | 0.784 | 9.18E+08 | 8.8E+08 | 1.04 |
| P03952 | Plasma kallikrein | 0.32 | 0.5714 | 0.784 | 5.4E+08 | 5.26E+08 | 1.03 |
| P07360 | Complement component C8 | 0.3 | 0.5837 | 0.793 | 3.22E+08 | 3.22E+08 | 1.00 |
| | gamma chain | | | | | | |
| P02750 | Leucine-rich alpha-2- | 0.29 | 0.5908 | 0.793 | 8.16E+08 | 7.95E+08 | 1.03 |
| | glycoprotein | | | | | | |
| Q15582 | Transforming growth factor- | 0.29 | 0.5922 | 0.793 | 22379758 | 23515537 | 0.95 |
| | beta-induced protein ig-h3 | | | | | | |
| P49908 | Selenoprotein P | 0.27 | 0.6054 | 0.803 | 97906499 | 92437854 | 1.06 |
| Q15610 | Extracellular matrix protein 1 | 0.26 | 0.5124 | 0.803 | 2.7E+08 | 2.68E+08 | 1.01 |
| P19825 | Inter-alpha-typsin inhibtor | 0.26 | 0.5141 | 0.803 | 3.18E+09 | 3.26E+09 | 0.97 |
| | heavy chain H2 | | | | | | |
| P19320 | Vascular cell adhesion protein | 0.23 | 0.629 | 0.814 | 19514434 | 21040364 | 0.93 |
| | 1 | | | | | | |
| P43251 | Biotinidase | 0.23 | 0.6327 | 0.814 | 1.3E+08 | 1.42E+08 | 0.91 |
| P01031 | Complement C5 | 0.19 | 0.6677 | 0.814 | 1.85E+09 | 1.89E+09 | 0.98 |
| Q86UX7 | Fermitin family homolog 3 | 0.18 | 0.6701 | 0.849 | 76129860 | 87451916 | 0.87 |
| P21333 | Filamin-A | 0.18 | 0.6763 | 0.850 | 38045207 | 40631822 | 0.94 |
| P00488 | Coagulation factor XIII A chain | 0.13 | 0.7166 | 0.892 | 1.84E+08 | 1.91E+08 | 0.96 |
| Q9UGM5 | Fetuin-B | 0.13 | 0.7198 | 0.892 | 1.65E+08 | 1.69E+08 | 0.98 |
| P04278 | Sex hormone-binding globulin | 0.11 | 0.7403 | 0.910 | 93647907 | 1.08E+08 | 0.86 |
| P30041 | Peroxiredoxin-6 | 0.09 | 0.7667 | 0.927 | 17144828 | 17396439 | 0.99 |
| P02745 | Complement C1q | 0.09 | 0.7703 | 0.927 | 7.05E+08 | 6.88E+08 | 1.02 |
| | subcomponent subunit B | | | | | | |
| P02679 | Fibrinogen gamma chain | 0.08 | 0.772 | 0.927 | 4.42E+08 | 4.32E+08 | 1.02 |
| Q9NPH3 | Interleukin-1 receptor | 0.08 | 0.7759 | 0.927 | 18337034 | 21353257 | 0.86 |
| | accessory protein | | | | | | |
| P00338 | L-lactate dehydrogenase A | 0.07 | 0.7934 | 0.941 | 9971161 | 9783411 | 1.02 |
| | chain | | | | | | |
| P07996 | Thrombospondin-1 | 0.06 | 0.8058 | 0.944 | 92835344 | 1.01E+08 | 0.91 |
| P25311 | Zinc-alpha-2-glycoprotein | 0.06 | 0.8074 | 0.944 | 1.72E+09 | 1.7E+09 | 1.01 |
| Q9Y490 | Talin-1 | 0.06 | 0.8149 | 0.946 | 1.4E+08 | 1.58E+08 | 0.89 |
| P0CG06 | Immunoglobulin lambda constant 3 | 0.05 | 0.8228 | 0.949 | 1.35E+08 | 1.54E+08 | 0.85 |
| Q96IY4 | Carboxypeptidase B2 | 0.05 | 0.8738 | 0.992 | 1.26E+08 | 1.31E+08 | 0.96 |
| O00533 | Neural cell adhesion molecule | 0.02 | 0.8774 | 0.992 | 11480232 | 11161656 | 1.03 |
| | L1-like protein | | | | | | |
| P08571 | Monocyte differentiation | 0.02 | 0.8899 | 0.992 | 1.27E+08 | 1.24E+08 | 1.03 |
| | antigen CD14 | | | | | | |
| P02765 | Alpha-2-HS-glycoprotein | 0.02 | 0.8933 | 0.992 | 1.46E+09 | 1.44E+09 | 1.01 |
| P01857 | Immunoglobulin heavy | 0.01 | 0.9034 | 0.992 | 1.56E+08 | 1.59E+08 | 0.98 |
| | constant gamma 1 | | | | | | |
| P01008 | Antithrombin-III | 0.01 | 0.9108 | 0.992 | 5.16E+09 | 5.1E+09 | 1.01 |
| P00915 | Carbonic anhydrase 1 | 0.01 | 0.9123 | 0.992 | 54151372 | 51702715 | 1.05 |
| Q6EMK4 | Vasorin | 0.01 | 0.9118 | 0.992 | 22619673 | 23533310 | 0.96 |
| O00391 | Sulfhydryl oxidase 1 | 0.01 | 0.9143 | 0.992 | 57356817 | 57950126 | 0.99 |
| P22259 | Coagulation factor V | 0.01 | 0.9247 | 0.993 | 3.68E+08 | 3.68E+28 | 1.00 |
| P07359 | Platelet glycoprotein Ib alpha | 0.01 | 0.9291 | 0.993 | 56061100 | 57453774 | 0.98 |
| | chain | | | | | | |
| P06727 | Apolipoprotein A-IV | 0.01 | 0.9391 | 0.993 | 4.54E+09 | 4.49E+09 | 1.01 |
| P26927 | Hepatocyte growth factor-like | 0 | 0.9457 | 0.993 | 1.21E+08 | 1.23E+08 | 0.98 |
| | protein | | | | | | |
| P13796 | Plastin-2 | 0 | 0.9503 | 0.993 | 34554768 | 34214663 | 1.01 |
| P63104 | 14-3-3 protein zeta/delta | 0 | 0.9538 | 0.993 | 28443038 | 28830798 | 0.99 |
| P01623 | Immunoglobulin kappa | 0 | 0.9579 | 0.993 | 25042231 | 27111840 | 0.92 |
| | variable 3-20 | | | | | | |
| P05154 | Plasma serine protease | 0 | 0.9629 | 0.993 | 1.55E+08 | 1.64E+08 | 0.95 |
| | inhibitor | | | | | | |
| P0C0L4 | Complement C4-A | 0 | 0.9748 | 0.998 | 1.73E+10 | 1.71E+10 | 1.01 |
| P22352 | Glutathione peroxidase 3 | 0 | 0.9805 | 0.998 | 1.13E+08 | 1.2E+08 | 0.94 |
| PL8206 | Vinculin | 0 | 0.9858 | 0.998 | 71329343 | 78664847 | 0.91 |
| Q9UK55 | Protein Z-dependent protease | 0 | 0.9926 | 0.999 | 68425526 | 69087635 | 0.99 |
| | inhibitor | | | | | | |
| P01009 | Alpha-1-antitrypsin | 0 | 0.9993 | 0.999 | 2.39E+08 | 2.77E+08 | 0.86 |

CHR-T: clinical high-risk participants who transitioned to first episode psychosis;
CHR-NT: clinical high-risk participants who did not transition;
FDR: false discovery rate

TABLE 7
Functional enrichment analyses:
6 KEGG pathways significantly enriched

| | | Fisher’s exact |
|---|---|---|
| | | test p |
| | | (adjusted |
| | Count in | for false |
| KEGG pathway | gene set | discovery rate) |
| Complement and | 13 of 78 | 2.23E−21 |
| coagulation cascades | | |
| Staphylococcus aureus infection | 6 of 51 | 5.29E−09 |
| Pertussis | 4 of 74 | 6.38E−05 |
| Cholesterol metabolism | 3 of 48 | 0.00047 |
| Systemic lupus erythematosus | 3 of 94 | 0.0025 |
| Prion diseases | 2 of 33 | 0.0058 |

TABLE 8
Results of enzyme-linked immunoassay (ELISA)
tests for CHR-T and CHR-NT in EU-GEI

| | CHR-T | | CHR-NT | | | |
|---|---|---|---|---|---|---|
| | mean (SD) | | mean (SD) | | t | p |
| Alpha-2-macroglobulin | 1173.1 | (459.1) | 11501.7 | (711.1) | 3.2202 | 0.0016 |
| (μg/ml) | | | | | | |
| Apolipoprotein E | 163751.3 | (47433.8) | 151740.6 | (50903.5) | −1.3449 | 0.1818 |
| (ng/ml)^{a} | | | | | | |
| Complement C1q | 82811.7 | (35347.1) | 80204.6 | (33535.8) | −0.4153 | 0.6789 |
| (ng/ml) | | | | | | |
| Complement C1r | 65008.9 | (27901.6) | 52803.9 | (18481.6) | −2.7099 | 0.0084 |
| (μg/ml) | | | | | | |
| Complement C4 | 495765.9 | (222274.7) | 482208.2 | (192019.3) | −0.3538 | 0.7243 |
| binding protein | | | | | | |
| (ng/ml) | | | | | | |
| Complement C8 | 58233.5 | (22885.2) | 55706.0 | (21938.2) | −0.6196 | 0.5370 |
| (ng/ml) | | | | | | |
| Complement factor H | 701713.1 | (207717.4) | 663292.1 | (199397.1) | −1.0112 | 0.3147 |
| (ng/ml)^{b} | | | | | | |
| Immunoglobulin M | 1752941.0 | (770671.5) | 1941142.0 | (936493.0) | 1.2460 | 0.2153 |
| (ng/ml) | | | | | | |
| Plasminogen | 206880.2 | (73232.4) | 176929.9 | (62709.8) | −2.3786 | 0.0196 |

(ng/ml)

Data available for 48 CHR-T and 84 CHR-NT, except for ^{a}Apolipoprotein E (46 CHR-T, 84 CHR-NT) and ^{b}Complement factor H (45 CHR-T, 82 CHR-NT).
Means (and standard deviations) are presented and are compared using 2-sided t-test with unequal variances.
CHR-T: clinical high-risk participants who transitioned to first episode psychosis; CHR-NT: clinical high-risk participants who did not transition; SD: standard deviation

TABLE 9
Comparison of characteristics for participants included in replication experiment
(N = 135) from total EU-GEI CHR cohort (N = 344)

| | | Included, N = 135 | Not included, N = 209 | | |
|---|---|---|---|---|---|
| | Missing | (49 CHR-T, | (CHR-T 16, | | |
| | data, n (%) | 86 CHR-NT) | CHR-NT 193) | t/χ² | p |
| Baseline age in | 0 | 22.2 (4.8) | 22.5 (5.0) | −0.570 | 0.569 |
| years, mean | | | | | |
| (SD) | | | | | |
| Sex, n (%) | 0 | 82 male (60.7%) | 103 male (49.3%) | 4.322 | 0.037 |
| | | 53 female (39.3%) | 106 female (50.7%) | | |
| Baseline BMI in | 50 (14.5%) | 23.7 (4.1) | 24.2 (5.8) | −0.936 | 0.350 |
| kg/m², mean | | | | | |
| (SD) | | | | | |
| Baseline years | 38 (11.0%) | 14.3 (2.9) | 14.5 (3.2) | −0.574 | 0.566 |
| in education, | | | | | |
| mean (SD) | | | | | |
| Ethnicity, n (%) | 0 | 93 white (68.9%) | 154 white (73.7%) | 1.030 | 0.597 |
| | | 14 black (10.4%) | 20 black (9.6%) | | |
| | | 28 other (20.7%) | 35 other (16.7%) | | |
| Ever used | 10 (2.9%) | 101 yes (74.8%) | 143 yes (68.4%) | 1.328 | 0.249 |
| cannabis, n (%) | | 31 no (23.0%) | 59 no (28.2%) | | |
| | | 3 not known (2.2%) | 7 not known (3.3%) | | |
| Baseline | 95 (27.6%) | 43 yes (31.9%) | 45 yes (21.5%) | 3.155 | 0.076 |
| cannabis use, n | | 60 no (44.4%) | 101 no (48.3%) | | |
| (%) | | 32 not known (23.7%) | 63 not known (30.1%) | | |
| Baseline | 38 (11.0%) | 72 yes (53.3%) | 89 yes (42.6%) | 2.893 | 0.089 |
| tobacco use, n | | 51 no (37.8%) | 94 no (45.0%) | | |
| (%) | | 12 not known (8.9%) | 26 not known (12.4%) | | |
| Baseline alcohol | 12 (3.5%) | 36 yes (26.7%) | 137 yes (65.6%) | 0.640 | 0.424 |
| use, n (%) | | 97 no (71.9%) | 62 no (29.7%) | | |
| | | 2 not known (1.5%) | 10 not known (4.8%) | | |
| Baseline | 86 (25.0%) | 52 yes (38.5%) | 77 yes (36.8%) | 0.065 | 0.799 |
| medication use, | | Antidepressant 30 | Antidepressant 44 | | |
| n (%) | | Antipsychotic 10 | Antipsychotic 10 | | |
| | | Hypnotic 3 | Hypnotic 11 | | |
| | | Other 9 | Other 12 | | |
| | | 50 no (37.0%) | 79 no (37.8%) | | |
| | | 33 not known (24.4%) | 53 not known (25.4%) | | |
| Baseline GAF | 27 (7.8%) | 54.4 (10.2) | 55.6 (10.0) | −1.103 | 0.271 |
| symptoms score, | | | | | |
| mean (SD) | | | | | |
| Baseline GAF | 12 (3.5%) | 55.5 (13.7) | 55.4 (11.3) | 0.006 | 0.996 |
| disability score, | | | | | |
| mean (SD) | | | | | |
| Baseline SANS | 45 (13.1%) | 17.1 (12.7) | 14.6 (10.8) | 1.784 | 0.075 |
| total composite | | | | | |
| score, mean | | | | | |
| (SD) | | | | | |
| Baseline SANS | 29 (8.4%) | 5.6 (3.8) | 5.4 (3.5) | 0.510 | 0.610 |
| total global | | | | | |
| score, mean | | | | | |
| (SD) | | | | | |
| Baseline BPRS | 25 (7.3%) | 44.9 (11.2) | 42.9 (9.6) | 1.650 | 0.100 |
| total score, | | | | | |
| mean (SD) | | | | | |
| Baseline | 16 (4.7%) | 18.8 (9.5) | 18.9 (8.9) | −0.126 | 0.900 |
| MADRS total | | | | | |
| score, mean | | | | | |
| (SD) | | | | | |
| 2 year GAF | 142 (41.3%) | 54.6 (15.0) | 63.0 (11.6) | −4.083 | <0.001 |
| symptoms score, | | | | | |
| mean (SD) | | | | | |
| 2 year GAF | 124 (36.0%) | 56.9 (15.0) | 63.6 (13.8) | −3.333 | 0.001 |
| disability score, | | | | | |
| mean (SD) | | | | | |
| 2 year GAF | 124 (36.0%) | 40 good (29.6%) | 72 good (34.4%) | 3.002 | 0.083 |
| disability score, | | 51 poor (37.8%) | 57 poor (27.2%) | | |
| dichotomous | | 44 not known (32.6%) | 80 not known (38.3%) | | |
| outcome ^{a} | | | | | |

^{a} Poor functioning: GAF disability score ≤60; good functioning: GAF disability score >60
Tobacco use was defined as daily use for at least 1 month over the previous 12 months. Alcohol use was defined as at least 12 or more alcoholic beverages over the previous 12 months. Missing data excluded in hypothesis tests.
EU-GEI: European Union Gene Environment Interaction study; CHR-T: clinical high risk, transitioned to psychosis; CHR-NT: clinical high risk, did not transition to psychosis; BMI: body mass index; GAF: General Assessment of Functioning; SANS: Scale for the Assessment of Negative Symptoms; BPRS: Brief Psychiatric Rating Scale; MADRS: Montgomery Asberg Depression Rating Scale

TABLE 10
Descriptive statistics for EU-GEI replication CHR-T and CHR-NT groups

| | Missing | | | | |
|---|---|---|---|---|---|
| | data, n | CHR-T | CHR-NT | | |
| | (%) | N = 49 | N = 86 | t/χ² | p |
| Baseline age | 0 | 22.0 (4.7) | 22.3 (4.9) | t = −0.339 | 0.735 |
| in years, | | | | | |
| mean (SD) | | | | | |
| Sex, n (%) | 0 | 26 male (53%) | 56 male (65%) | χ² = 1.902 | 0.168 |
| | | 23 female (47%) | 30 female (35%) | | |
| Baseline | 21 | 24.5 (4.5) | 23.2 (3.8) | t = 1.722 | 0.088 |
| BMI in | (15.6%) | | | | |
| kg/m², mean | | | | | |
| (SD) | | | | | |
| Baseline | 12 (8.8%) | 14.0 (3.1) | 14.3 (2.6) | t = −0.573 | 0.568 |
| years in | | | | | |
| education, | | | | | |
| mean (SD) | | | | | |
| Ethnicity, n | 0 | 31 white (63.3%) | 62 white (72.1%) | χ² = 8.549 | 0.014 |
| (%) | | 10 black (20.4%) | 4 black (4.7%) | | |
| | | 8 other (16.3%) | 20 other (23.3%) | | |
| Ever used | 3 (2.2%) | 35 yes (71.4%) | 66 yes (76.7%) | χ² = 0.170 | 0.680 |
| cannabis, n | | 12 no (24.5%) | 19 no (22.1%) | | |
| (%) | | 2 not known (4.1%) | 1 not known (1.2%) | | |
| Baseline | 32 | 14 yes (28.6%) | 29 yes (33.7%) | χ² = 0.186 | 0.666 |
| cannabis | (23.7%) | 22 no (44.9%) | 38 no (44.2%) | | |
| use, n (%) | | 13 not known (26.5%) | 19 not known | | |
| | | | (22.1%) | | |
| Baseline | 12 (8.9%) | 19 yes (38.8%) | 53 yes (61.6%) | χ² = 4.647 | 0.031 |
| tobacco use, | | 23 no (46.9%) | 28 no (32.6%) | | |
| n (%) | | 7 not known (14.3%) | 5 not known (5.8%) | | |
| Baseline | 2 (1.5%) | 34 yes (69.4%) | 63 yes (73.3%) | χ² = 0.168 | 0.682 |
| alcohol use, | | 14 no (28.6%) | 22 no (25.6%) | | |
| n (%) | | 1 not known (2.0%) | 1 not known (1.2%) | | |
| Baseline | 33 | 20 yes (40.8%) | 32 yes (37.2%) | χ² = 0.285 | 0.593 |
| medication | (24.4%) | Antidepressant 12 | Antidepressant 18 | | |
| use, n (%) | | Antipsychotic 6 | Antipsychotic 4 | | |
| | | Hypnotic 1 | Hypnotic 2 | | |
| | | Other 1 | Other 8 | | |
| | | 20 no (40.8%) | 30 no (34.9%) | | |
| | | 9 not known (18.4%) | 24 not known | | |
| | | | (27.9%) | | |
| Baseline | 4 (3.0%) | 53.0 (10.1) | 55.1 (10.2) | t = −1.089 | 0.278 |
| GAF | | | | | |
| symptoms | | | | | |
| score, mean | | | | | |
| (SD) | | | | | |
| Baseline | 4 (3.0%) | 53.0 (12.6) | 56.8 (14.1) | t = −1.531 | 0.128 |
| GAF | | | | | |
| disability | | | | | |
| score, mean | | | | | |
| (SD) | | | | | |
| Baseline | 15 | 20.9 (14.1) | 14.9 (11.4) | t = 2.389 | 0.019 |
| SANS total | (11.1%) | | | | |
| composite | | | | | |
| score, mean | | | | | |
| (SD) | | | | | |
| Baseline | 10 (7.4%) | 6.6 (4.1) | 5.0 (3.5) | t = 2.252 | 0.026 |
| SANS total | | | | | |
| global score, | | | | | |
| mean (SD) | | | | | |
| Baseline | 10 (7.4%) | 48.1 (11.2) | 43.1 (10.8) | t = 2.456 | 0.015 |
| BPRS total | | | | | |
| score, mean | | | | | |
| (SD) | | | | | |
| Baseline | 6 (4.4%) | 19.9 (10.2) | 18.1 (9.1) | t = 1.004 | 0.317 |
| MADRS | | | | | |
| total score, | | | | | |
| mean (SD) | | | | | |
| 2 year GAF | 49 | 43.6 (14.1) | 63.5 (10.6) | t = −7.281 | <0.001 |
| symptoms | (36.3%) | | | | |
| score, mean | | | | | |
| (SD) ^{a} | | | | | |
| 2 year GAF | 44 | 45.3 (9.5) | 65.1 (13.9) | t = −7.969 | <0.001 |
| disability | (32.6%) | | | | |
| score, mean | | | | | |
| (SD) ^{b} | | | | | |
| 2 year GAF | 44 | 28 poor functioning | 23 poor functioning | χ² = 25.261 | <0.001 |
| disability | (32.6%) | (57.1%) | (26.7%) | | |
| score, | | 2 good functioning | 38 good functioning | | |
| dichotomous | | (4.1%) | (44.2%) | | |
| outcome ^{c} | | 19 not known (38.8%) | 25 not known | | |
| | | | (29.1%) | | |

^{a} Data available for 86 of 135 participants (CHR-NT n = 27, CHR-T n = 59)
^{b} Data available for 91 of 135 participants (CHR-NT n = 30, CHR-T n = 61)
^{c} Poor functioning: GAF disability score ≤60; good functioning: GAF disability score >60
Tobacco use was defined as daily use for at least 1 month over the previous 12 months.
Alcohol use was defined as at least 12 or more alcoholic beverages over the previous 12 months.
Missing data excluded in hypothesis tests.
EU-GEI: European Union Gene Environment Interaction study; CHR-T: clinical high risk, transitioned to psychosis; CHR-NT: clinical high risk, did not transition to psychosis; BMI: body mass index; GAF: General Assessment of Functioning; SANS: Scale for the Assessment of Negative Symptoms; BPRS: Brief Psychiatric Rating Scale; MADRS: Montgomery Asberg Depression Rating Scale

TABLE 11
Results of ANCOVA and fold changes (CHR-T vs. CHR-NT) for proteins identified in EU-GEI replication
dataset (adjusted for age, sex, BMI, years in education, tobacco use and ethnicity)

| | | | | | | Mean LFQ, | |
|---|---|---|---|---|---|---|---|
| | | | | | Mean LFQ, | non- | |
| | | | | | transition | transition | Mean |
| | | | | 5% FDR- | group | group | ratio T |
| Uniprot no. | Protein name | F | p | adjusted p | (n = 49) | (n = 86) | vs NT |
| P01023 | Alpha-2-macroglobulin | 264.41 | 9.81E−33 | 1.17E−30 | 1.48E+10 | 6.14E+10 | 0.24 |
| A0A075B6N9 | Immunoglobulin heavy | 109.92 | 7.11E−19 | 4.23E−17 | 1.19E+09 | 3.58E+09 | 0.33 |
| | constant mu | | | | | | |
| P00747 | Plasminogen | 78.47 | 6.43E−15 | 2.55E−13 | 3.68E+10 | 2.83E+10 | 1.30 |
| P10909 | Clusterin | 73341 | 3.17E−14 | 9.44E−13 | 1.28E+10 | 9.43E+09 | 1.36 |
| P01011 | Alpha-1-antichymotrypsin | 68.88 | 1.38E−13 | 3.28E−12 | 3.57E+10 | 2.58E+10 | 1.38 |
| G3XAW2 | Complement factor I | 65.48 | 4.24E−13 | 8.4E−12 | 9.37E+09 | 7.38E+09 | 1.27 |
| A0A075B6L0 | Immunoglobulin lambda | 58.29 | 4.90E−12 | 8.33E−11 | 1.24E+09 | 2.22E+09 | 0.56 |
| | constant 3 | | | | | | |
| A0A075B6N8 | Immunogloblin heavy | 56.09 | 1.06E−11 | 1.42E−10 | 4.02E+09 | 7.71E+09 | 0.52 |
| | constant gamma 3 | | | | | | |
| P08603 | Complement factor H (H | 56.05 | 1.08E−11 | 1.42E−10 | 4.92E+10 | 4.18E+10 | 1.18 |
| | factor 1) | | | | | | |
| P13671 | Complement component C6 | 55.01 | 1.55E−11 | 1.7E−10 | 6.58E+09 | 4.74E+09 | 1.39 |
| P04004 | Vitronectin | 54.97 | 1.57E−11 | 1.7E−10 | 2.17E+10 | 1.63E+10 | 1.33 |
| P22792 | Carboxypeptidase N subunit | 52.23 | 4.18E−11 | 4.15E−10 | 3.69E+09 | 2.85E+09 | 1.29 |
| | 2 | | | | | | |
| P07360 | Complement component C8 | 49.11 | 1.30E−10 | 1.19E−09 | 4.35E+09 | 3.4E+09 | 1.28 |
| | gamma chain | | | | | | |
| P08571 | Monocyte differentation | 47.31 | 2.52E−10 | 2.14E−09 | 5.51E+08 | 3.95E+08 | 1.39 |
| | antigen CD14 | | | | | | |
| P01031 | Complement C5 | 44.69 | 6.71E−10 | 5.32E−29 | 1.36E+10 | 1.14E+10 | 1.19 |
| B7ZXJ8 | ITIH4 protein | 43.28 | 1.14E−09 | 8.51E−29 | 3.08E+10 | 2.55E+10 | 1.21 |
| P07359 | Platelet glycoprotein Ib | 35.1 | 2.79E−08 | 1.96E−07 | 2.84E+08 | 2.03E+08 | 1.40 |
| | alpha chain | | | | | | |
| P15169 | Carboxypeptidase N catalytic | 33.9 | 4.53E−08 | 2.99E−07 | 1.75E+09 | 1.32E+09 | 1.33 |
| | chain (CPN) | | | | | | |
| P36955 | Pigment epithelium-derived | 32.23 | 8.97E−08 | 5.62E−07 | 6.33E+09 | 5.2E+09 | 1.22 |
| | factor | | | | | | |
| P07357 | Complement component C8 | 31.63 | 1.15E−07 | 6.82E−07 | 3.98E+09 | 3.18E+09 | 1.25 |
| | alpha chain | | | | | | |
| P27169 | Serum | 30.66 | 1.71E−07 | 9.69E−07 | 9.48E+08 | 7.27E+08 | 1.30 |
| | paraoxonase/arylesterase 1 | | | | | | |
| Q96IY4 | Carboxypeptidase B2 | 29.85 | 2.39E−07 | 1.29E−06 | 9.03E+08 | 6.39E+08 | 1.41 |
| Q16610 | Extracellular matrix protein 1 | 26.68 | 9.12E−07 | 4.72E−06 | 2.42E+09 | 1.75E+09 | 1.38 |
| P04003 | C4b-binding protein alpha | 26.26 | 1.09E−06 | 5.41E−06 | 5.2E+09 | 6.76E+09 | 0.77 |
| | chain | | | | | | |
| P12259 | Coagulation factor V | 24.43 | 2.40E−06 | 1.14E−05 | 1.12E+09 | 8.61E+03 | 1.30 |
| P06727 | Apolipoprotein A-IV | 23.96 | 2.95E−06 | 1.35E−05 | 4.86E+10 | 4.2E+10 | 1.16 |
| P80108 | Phosphatidylinositol-glycan- | 22.81 | 4.87E−06 | 2.15E−05 | 1.25E+09 | 9.86E+08 | 1.27 |
| | specific phospholipase D (PI- | | | | | | |
| | G PLD) | | | | | | |
| P05155 | Plasma protease C1 Inhibitor | 22.67 | 5.19E−06 | 2.2E−05 | 1.61E+10 | 1.3E+10 | 1.23 |
| P19827 | Inter-alpha-trypsin inhibitor | 20.64 | 1.28E−05 | 5.25E−05 | 2.21E+10 | 1.92E+10 | 1.15 |
| | heavy chain H1 | | | | | | |
| P0C0L4 | Complement C4-A (Acidic | 19.53 | 2.11E−05 | 8.37E−05 | 3.94E+08 | 6.91E+08 | 0.57 |
| | complement C4) | | | | | | |
| B4E1Z4 | cDNA FLI55673 | 18.98 | 2.71E−05 | 0.000104 | 3.8E+10 | 3.36E+10 | 1.13 |
| P51884 | Lumican | 18.9 | 2.81E−05 | 0.000104 | 3.37E+09 | 2.68E+09 | 1.26 |
| P00736 | Complement C1r | 18.64 | 3.17E−05 | 0.000114 | 5.36E+09 | 4.75E+09 | 1.13 |
| | subcomponent | | | | | | |
| O43866 | CD5 antigen-like | 18.47 | 3.42E−05 | 0.00012 | 2.39E+08 | 3.66E+08 | 0.65 |
| P19823 | Inter-alpha-trypsin inhibitor | 15.64 | 0.0001 | 0.000313 | 4.08E+10 | 3.68E+10 | 1.11 |
| | heavy chain H2 | | | | | | |
| P06396 | Gelsolin | 16.15 | 0.0001 | 0.000313 | 1.72E+10 | 1.49E+10 | 1.15 |
| P02765 | Alpha-2-HS-glycoprotein | 17.11 | 0.0001 | 0.000313 | 2.82E+10 | 2.44E+10 | 1.16 |
| Q04756 | Hepatocyte growth factor | 16.22 | 0.0001 | 0.000313 | 5.53E+08 | 4.65E+03 | 1.13 |
| | activator | | | | | | |
| O00391 | Sulfhydryl oxidase 1 | 14.82 | 0.0002 | 0.00061 | 3.36E+08 | 2.86E+03 | 1.18 |
| P02748 | Complement component C9 | 13.25 | 0.0004 | 0.00119 | 8.05E+09 | 6.74E+09 | 1.19 |
| P00738 | Haptoglobin | 11.99 | 0.0007 | 0.001983 | 2.02E+09 | 4.13E+09 | 0.49 |
| P02790 | Hemopexin | 11.97 | 0.0007 | 0.001983 | 7.36E+10 | 6.69E+10 | 1.10 |
| P08697 | Alpha-2-antiplasmm | 11.36 | 0.001 | 0.002705 | 1.05E+10 | 9.47E+09 | 1.10 |
| P23142 | Fibulin-1 | 11.37 | 0.001 | 0.002705 | 1.15E+09 | 8.44E−08 | 1.37 |
| P05452 | Tetranectin (TN) | 11.15 | 0.0011 | 0.002909 | 3.22E+09 | 2.79E+09 | 1.15 |
| P06681 | Complement C2 | 10.96 | 0.0012 | 0.003104 | 1.18E+09 | 9.97E+08 | 1.18 |
| P02751 | Fibronectin | 10.62 | 0.0014 | 0.003545 | 5.96E+10 | 4.3E+10 | 1.39 |
| P07558 | Complement component C8 | 10.24 | 0.0017 | 0.004129 | 2.98E+03 | 2.53E+09 | 1.18 |
| | beta chain | | | | | | |
| P02649 | Apolipoprotein E | 10.26 | 0.0017 | 0.004129 | 7.3E+09 | 6.04E+09 | 1.21 |
| P01834 | Immunoglobulin kappa | 10.09 | 0.0019 | 0.004433 | 1.96E+03 | 2.65E+09 | 0.74 |
| | constant | | | | | | |
| O75882 | Attractin | 10.07 | 0.0019 | 0.004433 | 1.55E+03 | 1.31E+09 | 1.19 |
| P00734 | Prothrombin | 9.98 | 0.002 | 0.004577 | 3.74E+10 | 3.43E+10 | 1.03 |
| P17936 | Insulin-like growth factor- | 9.46 | 0.0026 | 0.005838 | 8.49E+08 | 6.51E+03 | 1.30 |
| | binding protein 3 | | | | | | |
| P00746 | Complement factor D | 9.15 | 0.003 | 0.006611 | 2.18E+08 | 1.77E+08 | 1.23 |
| P02675 | Fibrinogen beta chain | 8.48 | 0.0043 | 0.005304 | 4.98E+09 | 5.74E+09 | 0.87 |
| P00488 | Coagulation factor XIII A | 8.32 | 0.0046 | 0.005775 | 1.03E+03 | 8.21E+08 | 1.26 |
| | chain | | | | | | |
| P43652 | Afamin | 8.04 | 0.0053 | 0.011065 | 1.65E+10 | 1.5E+10 | 1.10- |
| P35858 | Insulin-like growth factor- | 7.33 | 0.0077 | 0 015758 | 6.06E+09 | 5.47E+09 | 1.11 |
| | binding protein complex acid | | | | | | |
| | labile subunit (ALS) | | | | | | |
| P05543 | T4-binding globulin | 7.3 | 0.0079 | 0.015934 | 1.13E+09 | 8.9E+08 | 1.27 |
| P10643 | Complement component C7 | 7.09 | 0.0088 | 0.017453 | 2.99E+09 | 2.31E+09 | 1.25 |
| P02787 | Serotransferrin | 6.92 | 0.0096 | 0.018728 | 6.56E+09 | 7.9E+09 | 0.83 |
| P26927 | Hepatocyte growth factor- | 6.8 | 0.0102 | 0 019267 | 4.68E+08 | 3.79E+08 | 1.24 |
| | like protein (Macrophage | | | | | | |
| | stimulatory protein) | | | | | | |
| P18428 | Lipopolysaccharide-binding | 6.81 | 0.0102 | 0.019267 | 5.69E+08 | 4.48E+08 | 1.27 |
| | protein (LBP) | | | | | | |
| P02760 | Protein AMBP | 6.63 | 0.0112 | 0.020825 | 1.16E+10 | 1.1E+10 | 1.05 |
| P00450 | Ceruloplasmin | 6.38 | 0.0128 | 0.023434 | 4.58E+10 | 4.92E+10 | 0.93 |
| P05154 | Plasma serine protease | 6.25 | 0.0137 | 0.024702 | 7.96E+08 | 6.93E+08 | 1.15 |
| | inhibitor | | | | | | |
| P02766 | Transthyretin | 6.14 | 0.0145 | 0.025754 | 4.93E+08 | 6.05E+08 | 0.82 |
| Q14520-2 | Hyaluronan-binding protein | 6.09 | 0.0149 | 0.026042 | 1.38E+09 | 1.15E+09 | 1.20 |
| | 2 | | | | | | |
| P02679-2 | Fibrinogen gamma chain | 6.07 | 0.0151 | 0.026042 | 7.28E+09 | 8.08E+09 | 0.90 |
| P00740 | Coagulation factor IX | 5.32 | 0.0164 | 0.02788 | 8.19E+08 | 7.41E+08 | 1.10 |
| Q96XN2 | Beta-Ala-His dipeptidase | 5.82 | 0.0173 | 0.023956 | 8.09E+08 | 6.35E+08 | 1.27 |
| P01019 | Angiotensinogen | 5.42 | 0.0215 | 0.035535 | 1.29E+10 | 1.15E+10 | 1.12 |
| P04114 | Apolipoprotein B-100 | 5.29 | 0.0231 | 0.037656 | 9.92E+13 | 9.3E+10 | 1.07 |
| P02647 | Apolipoprotein A-I | 5.03 | 0.0266 | 0.042776 | 6.21E+09 | 6.98E+09 | 0.89 |
| P05546 | Heparin cofactor 2 | 4.81 | 0.0302 | 0.047917 | 1.36E+10 | 1.29E+10 | 1.06 |
| P01009 | Alpha-1-antitryosin | 4.77 | 0.0307 | 0.04807 | 1.48E+09 | 1.86E+09 | 0.79 |
| P04275 | von Willebrand factor | 4.71 | 0.0315 | 0.0493 | 4.87E+08 | 3.55E+08 | 1.37 |
| P02743 | Serum amyloid P-component | 4.68 | 0.0324 | 0.049431 | 6.6E+03 | 5.42E+09 | 1.22 |
| | (SAP) | | | | | | |
| P01857 | Immunoglobulin heavy | 4.63 | 0.0334 | 0.050311 | 2.3E+09 | 2.02E+09 | 1.14 |
| | constant gamma 1 | | | | | | |
| P01008 | Antithrombin-III | 4.27 | 0.0408 | 0.06069 | 2.16E+13 | 2.38E+10 | 0.91 |
| P25311 | Zinc-alpha-2-glyprotein | 4.08 | 0.0456 | 0.066953 | 1.41E+10 | 1.39E+10 | 1.01 |
| P05160 | Coagulation factor XIII B chain | 3.4 | 0.0675 | 0.097957 | 1.21E+09 | 1.14E+09 | 1.06 |
| P22891 | Vitamin K-dependent protein | 3.13 | 0.0795 | 0.113982 | 2.6E+08 | 2.35E+08 | 1.10 |
| | Z | | | | | | |
| P02746 | Complement C1q | 2.85 | 0.0936 | 0.1326 | 3.71E+09 | 3.17E+09 | 1.17 |
| | subcomponent subunit B | | | | | | |
| P0C0L5 | Complement C4-B (Basic | 2.72 | 0.1014 | 0.14196 | 9.89E+10 | 9.03E+10 | 1.09 |
| | complement C4) | | | | | | |
| Q96PD5 | N-acetylmuramoyl-L-alanine | 2.63 | 0.1075 | 0.14875 | 5.13E+09 | 4.87E+09 | 1.05 |
| | amidase | | | | | | |
| P01876 | Immunoglobulin heavy | 2.54 | 0.1135 | 0.154566 | 1.97E+09 | 2.73E+09 | 0.72 |
| | constant alpha 1 | | | | | | |
| P02763 | Alpha-1-acid glycoprotein 1 | 2.51 | 0.1154 | 0.154566 | 8.56E+08 | 1.02E+09 | 0.84 |
| P01042 | Kininogen-1 | 2.51 | 0.1156 | 0.154566 | 2.64E+10 | 2.55E+10 | 1.03 |
| K7ERI9 | Apolipoprotein C-I | 2.17 | 0.1433 | 0.189474 | 1.06E+09 | 1.26E+09 | 0.84 |
| Q06033 | Inter-alpha-trypsin inhibitor | 2.07 | 0.1524 | 0.199292 | 1.79E+09 | 1.59E+09 | 1.13 |
| | heavy chain H3 | | | | | | |
| P02750 | Leucine-rich alpha-2- | 2.01 | 0.1589 | 0.205534 | 5.42E+09 | 5.03E+09 | 1.08 |
| | glycoprotein | | | | | | |
| D6RF35 | Vitamin D-binding protein | 1.89 | 0.1721 | 0.220214 | 4.27E+10 | 4.19E+10 | 1.02 |
| P00748 | Coagulation factor XII | 1.85 | 0.1762 | 0.223062 | 4.2E+09 | 4.54E+09 | 0.93 |
| P09871 | Complement C1q | 1.7 | 0.1951 | 0.244388 | 4.23E+09 | 4.58E+09 | 0.92 |
| | subcomponent | | | | | | |
| P07996 | Thrombospondin-1 | 1.11 | 0.2932 | 0.363446 | 4.62E+08 | 4.19E+08 | 1.10- |
| Q9NZP8 | Complement C1r | 1.06 | 0.3051 | 0.374298 | 3.33E+08 | 2.99E+08 | 1.12 |
| | subcomponent-like protein | | | | | | |
| P02747 | Complement C1q | 1.02 | 0.3148 | 0.382257 | 3.96E+09 | 3.78E+09 | 1.05 |
| | subcomponent subunit C | | | | | | |
| P04278 | Sex hormone-binding | 0.64 | 0.4244 | 0.507178 | 7.31E+08 | 6.27E+08 | 1.17 |
| | globulin | | | | | | |
| P29622 | Kallistatin (Kallikrein inhibitor) | 0.64 | 0.4262 | 0.507178 | 3.1E+09 | 3.04E+09 | 1.02 |
| P03952 | Plasma kallikrein | 0.58 | 0.4476 | 0.52737 | 4.4E+09 | 4.06E+09 | 1.08 |
| P43251 | Biotinidase (Biotinase) | 0.55 | 0.4607 | 0.537483 | 4.38E+08 | 4.78E+08 | 0.92 |
| P01024 | Complement C3 | 0.45 | 0.5025 | 0.577493 | 2.13E+09 | 2.11E+09 | 1.01 |
| P04196 | Histidine-rich glycoprotein | 0.45 | 0.5047 | 0.577493 | 2.15E+10 | 1.13E+10 | 1.02 |
| P02656 | Apolipoprotein C-III | 0.43 | 0.5145 | 0.5831 | 2.95E+09 | 2.7E+09 | 1.09 |
| O75636 | Ficolin-3 | 0.36 | 0.5475 | 0.614646 | 1.74E+09 | 1.58E+09 | 1.10 |
| P27918 | Properdin | 0.3 | 0.5819 | 0.64716 | 7.22E+08 | 6.85E+08 | 1.05 |
| P00742 | Coagulation factor X | 0.27 | 0.607 | 0.668824 | 1.04E+09 | 9.82E+08 | 1.06 |
| P02749 | Beta-2-glycoprotein 1 | 0.23 | 0.6357 | 0.694021 | 1.96E+10 | 1.95E+10 | 1.01 |
| P06276 | Acylcholine acylhydrolase | 0.2 | 0.6588 | 0.712702 | 5.34E+08 | 5.29E+08 | 1.01 |
| P08185 | Corticosteroid-binding | 0.13 | 0.717 | 0.768676 | 1.72E+09 | 1.71E+09 | 1.00 |
| | globulin | | | | | | |
| P02671 | Fibrinogen alpha chain | 0.08 | 0.7845 | 0.833531 | 6.62E+09 | 6.82E+09 | 0.97 |
| P03951 | Coagulation factor XI | 0.05 | 0.8228 | 0.86275 | 2.95E+08 | 2.63E+08 | 1.12 |
| P19652 | Alpha-1-acid glycoprotein 2 | 0.05 | 0.8265 | 0.86275 | 1.44E+08 | 1.48E+08 | 0.97 |
| | (AGP 2) | | | | | | |
| Q9UK55 | Protein Z-dependent protease inhibitor | 0.04 | 0.8393 | 0.868493 | 5.16E+08 | 5.13E+08 | 1.01 |
| P07225 | Vitamin K-dependent protein | 0.03 | 0.8521 | 0.874137 | 2.7E+09 | 2.7E+09 | 1.03 |
| | S | | | | | | |
| Q5VY30 | Retinol-binding protein | 0.02 | 0.8787 | 0.893721 | 3E+09 | 3.08E+09 | 0.97 |
| P68871 | Hemogloblin subunit beta | 0.02 | 0.9022 | 0.9079 | 1.09E+09 | 1.03E+09 | 1.06 |
| P22352 | Glutathione peroxidase 5 | 0.01 | 0.9079 | 0.9079 | 3.78E+08 | 3.68E+08 | 1.03 |
| | (GPx-3) | | | | | | |

CHR-T: clinical high-risk participants who transitioned to first episode psychosis;
CHR-NT: clinical high-risk participants who did not transition;
FDR: false discovery rate

TABLE 12
Ten percent highest-weighted features for support
vector machine models in EU-GEI replication
dataset (Model 4) according to mean feature weight
for models selected in cross-validation inner loop
Model 4: Replication model
(119 proteomic and 65 clinical features)

| | Mean |
|---|---|
| Feature | weight |
| Alpha-2-macroglobulin | −0.285 |
| Carboxypeptidase N subunit 2 | 0.214 |
| Complement C1s subcomponent | −0.187 |
| Immunoglobulin heavy constant mu | −0.173 |
| Alpha 1 anti-chymotrypsin | 0.170 |
| Plasminogen | 0.163 |
| Zinc alpha-2-glycoprotein | −0.162 |
| Clusterin | 0.162 |
| C4b binding protein alpha chain | −0.157 |
| Monocyte differentiation antigen CD14 | 0.155 |
| Extracellular matrix protein 1 | 0.142 |
| Attractin | 0.128 |
| Complement component 6 | 0.126 |
| Complement factor 1 | 0.119 |
| BPRS: bizarre behaviour | 0.112 |
| Immunoglobulin lambda constant 3 | −0.111 |
| Ceruloplasmin | −0.109 |
| Antithrombin III | −0.109 |

TABLE 13
Descriptive statistics for ALSPAC cases and controls

| | Cases | Controls | | |
|---|---|---|---|---|
| | N = 55 | N = 66 | t/χ² | p |
| Sex, n (%) | 22 male (40.0%) | 39 male (59.1%) | χ² = 4.374 | 0.036 |
| | 33 female (60.0%) | 27 female (40.9%) | | |
| BMI age 12 | 18.1 (2.8) | 17.7 (2.5) | t = 0.749 | 0.455 |
| in kg/m², | | | | |
| mean (SD) | | | | |
| Ethnicity | 50 white (90.9%) | 63 white (95.5%) | χ² = 0.729 | 0.202 |
| | 5 other/not known | 3 other/not known | | |
| | (9.1%) | (4.5%) | | |
| Maternal | 40 non-manual | 44 non-manual | χ² = 0.005 | 0.946 |
| social | (72.7%) | (66.7%) | | |
| class | 7 manual (12.7%) | 8 manual (12.1%) | | |
| | 8 not known | 14 not known | | |
| | (14.6%) | (21.2%) | | |

Cases: participants with no PEs age 12 and definite PEs age 18; Controls: participants with no PEs age 12 and no PEs age 18
PEs: psychotic experiences; BMI: body mass index
Missing data excluded in hypothesis tests.

TABLE 14
Results of ANCOVA and fold changes (definite PEs at 18 vs. no PEs at 18) for proteins
identified in ALSPAC age 12 plasma samples (adjusted for sex, BMI and maternal social class)

| | | | | | Fold change |
|---|---|---|---|---|---|
| Uniprot | | | | Adjusted p | (PE vs. no |
| No. | Protein name | F | p | (FDR 5%) | PE) |
| P04003 | C4b-binding protein alpha chain | 24.59 | 2.44E−06 | 0.000647 | 0.77 |
| P27169 | Serum paraoxonase/arylesterase 1 | 17.78 | 4.94E−05 | 0.006544 | 0.80 |
| P01871 | Immunoglobulin heavy constant mu | 16.04 | 0.0001 | 0.006625 | 0.78 |
| P55103 | Inhibin beta C chain | 16.68 | 0.0001 | 0.006625 | 1.31 |
| P10909 | Clusterin | 12.71 | 0.0005 | 0.0265 | 0.92 |
| P01591 | Immunoglobulin J chain | 9.69 | 0.0023 | 0.1007 | 0.70 |
| P01860 | Immunoglobulin heavy constant gamma 3 | 9.25 | 0.0029 | 0.1007 | 0.80 |
| P0DOY3 | Immunoglobulin lambda constant 3 | 8.9 | 0.0035 | 0.1007 | 0.81 |
| P07225 | Vitamin K-dependent protein S | 8.83 | 0.0036 | 0.1007 | 0.90 |
| Q03591 | Complement factor H-related protein 1 | 8.74 | 0.0038 | 0.1007 | 0.79 |
| P01023 | Alpha-2-macroglobulin | 7.92 | 0.0057 | 0.137318 | 0.85 |
| P01623 | Immunoglobulin kappa variable 3-20 | 7.49 | 0.0072 | 0.159 | 0.81 |
| P24593 | Insulin-like growth factor-binding protein 5 | 7.34 | 0.0078 | 0.159 | 1.26 |
| P01019 | Angiotensinogen | 6.74 | 0.0106 | 0.194333 | 0.91 |
| P26038 | Moesin | 6.68 | 0.011 | 0.194333 | 1.16 |
| P04040 | Catalase | 6.53 | 0.0119 | 0.197094 | 1.36 |
| P12109 | Collagen alpha-1 | 6.36 | 0.013 | 0.19875 | 1.16 |
| P08571 | Monocyte differentiation antigen CD14 | 6.29 | 0.0135 | 0.19875 | 0.91 |
| B9A064 | Immunoglobulin lambda-like polypeptide 5 | 6.03 | 0.0155 | 0.210675 | 0.85 |
| P09871 | Complement C1s subcomponent | 5.99 | 0.0159 | 0.210675 | 0.93 |
| P08697 | Alpha-2-antiplasmin | 5.5 | 0.0207 | 0.249542 | 0.94 |
| P15151 | Poliovirus receptor | 5.44 | 0.0214 | 0.249542 | 1.16 |
| P01717 | Immunoglobulin lambda variable 3-25 | 5.34 | 0.0226 | 0.249542 | 0.85 |
| Q12884 | Prolyl endopeptidase FAP | 5.34 | 0.0226 | 0.249542 | 1.24 |
| P00746 | Complement factor D | 5.16 | 0.0249 | 0.255364 | 1.12 |
| P01615 | Immunoglobulin kappa variable 2D-28 | 5.1 | 0.0258 | 0.255364 | 0.84 |
| P02671 | Fibrinogen alpha chain | 5.05 | 0.0266 | 0.255364 | 0.89 |
| P23142 | Fibulin-1 | 4.99 | 0.0275 | 0.255364 | 1.36 |
| P01834 | Immunoglobulin kappa constant | 4.95 | 0.028 | 0.255364 | 0.90 |
| P24592 | Insulin-like growth factor-binding protein 6 | 4.85 | 0.0297 | 0.255364 | 1.15 |
| P02679 | Fibrinogen gamma chain | 4.77 | 0.031 | 0.255364 | 0.89 |
| P02675 | Fibrinogen beta chain | 4.76 | 0.0311 | 0.255364 | 0.90 |
| P03951 | Coagulation factor XI | 4.72 | 0.0318 | 0.255364 | 0.91 |
| P80748 | Immunoglobulin lambda variable 3-21 | 4.63 | 0.0334 | 0.260324 | 0.85 |
| P55290 | Cadherin-13 | 4.43 | 0.0374 | 0.283171 | 0.88 |
| P02652 | Apolipoprotein A-II | 4.37 | 0.0388 | 0.285611 | 0.85 |
| P07358 | Complement component C8 beta chain | 4.12 | 0.0446 | 0.306075 | 1.08 |
| D6RAR4 | Hepatocyte growth factor activator | 4.11 | 0.045 | 0.306075 | 0.89 |
| P15144 | Aminopeptidase N | 4.07 | 0.046 | 0.306075 | 1.06 |
| Q99878 | Histone H2A type 1-J | 4.06 | 0.0462 | 0.306075 | 1.37 |
| P08294 | Extracellular superoxide dismutase [Cu—Zn] | 3.91 | 0.0505 | 0.326402 | 1.13 |
| P02750 | Leucine-rich alpha-2-glycoprotein | 3.83 | 0.0528 | 0.333143 | 0.92 |
| P61626 | Lysozyme C | 3.61 | 0.06 | 0.368644 | 0.91 |
| P12111 | Collagen alpha-3 | 3.55 | 0.0621 | 0.368644 | 1.09 |
| P00915 | Carbonic anhydrase 1 | 3.53 | 0.0626 | 0.368644 | 1.24 |
| P14151 | L-selectin | 3.35 | 0.07 | 0.403261 | 1.08 |
| O43866 | CD5 antigen-like | 3.26 | 0.0737 | 0.415543 | 0.86 |
| P02654 | Apolipoprotein C-I | 3.14 | 0.0791 | 0.421962 | 0.91 |
| P01024 | Complement C3 | 3.09 | 0.0814 | 0.421962 | 0.94 |
| P29622 | Kallistatin | 3.08 | 0.082 | 0.421962 | 1.11 |
| P02647 | Apolipoprotein A-I | 3.07 | 0.0823 | 0.421962 | 0.86 |
| Q14624 | Inter-alpha-trypsin inhibitor heavy chain H4 | 3.06 | 0.0828 | 0.421962 | 0.91 |
| O00187 | Mannan-binding lectin serine protease 2 | 2.92 | 0.0901 | 0.440382 | 1.07 |
| P16403 | Histone H1.2 | 2.91 | 0.0907 | 0.440382 | 1.19 |
| P06276 | Cholinesterase | 2.9 | 0.0914 | 0.440382 | 1.03 |
| Q9NQ79 | Cartilage acidic protein 1 | 2.84 | 0.0946 | 0.443526 | 1.08 |
| Q9H4A9 | Dipeptidase 2 | 2.83 | 0.0954 | 0.443526 | 0.84 |
| H0Y755 | Low affinity immunoglobulin gamma Fc | 2.74 | 0.1008 | 0.460552 | 1.11 |
| | region receptor III-A | | | | |
| Q9UBQ6 | Exostosin-like 2 | 2.5 | 0.1164 | 0.514185 | 1.08 |
| Q9UGM5 | Fetuin-B | 2.48 | 0.1178 | 0.514185 | 0.94 |
| O75636 | Ficolin-3 | 2.47 | 0.1191 | 0.514185 | 1.07 |
| O95497 | Pantetheinase | 2.45 | 0.1203 | 0.514185 | 1.15 |
| P43251 | Biotinidase | 2.38 | 0.1254 | 0.52197 | 1.06 |
| Q08380 | Galectin-3-binding protein | 2.36 | 0.1276 | 0.52197 | 1.08 |
| P63261 | Actin, cytoplasmic 2 | 2.34 | 0.1289 | 0.52197 | 1.10 |
| Q9Y5Y7 | Lymphatic vessel endothelial hyaluronic | 2.33 | 0.13 | 0.52197 | 1.09 |
| | acid receptor l | | | | |
| P00747 | Plasminogen | 2.26 | 0.1358 | 0.536145 | 1.07 |
| P22792 | Carboxypeptidase N subunit 2 | 2.21 | 0.1395 | 0.536145 | 0.95 |
| P01033 | Metalloproteinase inhibitor 1 | 2.21 | 0.1396 | 0.536145 | 0.87 |
| Q9ULI3 | Protein HEG homolog 1 | 2.11 | 0.1488 | 0.563314 | 1.13 |
| Q96PD5 | N-acetylmuramoyl-L-alanine amidase | 2.06 | 0.1539 | 0.56376 | 1.09 |
| P05019 | Insulin-like growth factor I | 2.06 | 0.154 | 0.56376 | 1.24 |
| P35858 | Insulin-like growth factor-binding protein | 2.05 | 0.1553 | 0.56376 | 1.10 |
| | complex acid labile subunit | | | | |
| P02753 | Retinol-binding protein 4 | 2.01 | 0.1592 | 0.570108 | 1.13 |
| P02452 | Collagen alpha-1 (I) chain | 1.9 | 0.1706 | 0.592292 | 1.18 |
| P39060 | Collagen alpha-1 (XVIII) chain | 1.9 | 0.1707 | 0.592292 | 1.06 |
| P02747 | Complement C1q subcomponent subunit C | 1.89 | 0.1721 | 0.592292 | 0.96 |
| P04004 | Vitronectin | 1.78 | 0.1853 | 0.629545 | 1.06 |
| P07942 | Laminin subunit beta-1 | 1.75 | 0.1887 | 0.632981 | 1.01 |
| P02745 | Complement C1q subcomponent subunit A | 1.7 | 0.1944 | 0.64395 | 0.93 |
| P07359 | Platelet glycoprotein Ib alpha chain | 1.65 | 0.201 | 0.653451 | 1.03 |
| P07737 | Profilin-1 | 1.65 | 0.2022 | 0.653451 | 1.17 |
| P00751 | Complement factor B | 1.58 | 0.2116 | 0.67559 | 0.97 |
| H0YD13 | CD44 antigen | 1.53 | 0.2185 | 0.681773 | 1.09 |
| P48740 | Mannan-binding lectin serine protease 1 | 1.53 | 0.2192 | 0.681773 | 0.91 |
| P07357 | Complement component C8 alpha chain | 1.5 | 0.2227 | 0.681773 | 1.05 |
| P08603 | Complement factor H | 1.48 | 0.2263 | 0.681773 | 1.04 |
| Q16706 | Alpha-mannosidase 2 | 1.48 | 0.2264 | 0.681773 | 0.92 |
| P20851 | C4b-binding protein beta chain | 1.46 | 0.229 | 0.681854 | 0.89 |
| Q12913 | Receptor-type tyrosine-protein phosphatase eta | 1.43 | 0.2341 | 0.686962 | 0.93 |
| Q15582 | Transforming growth factor-beta-induced | 1.42 | 0.2359 | 0.686962 | 1.04 |
| | protein ig-h3 | | | | |
| Q6UXB8 | Peptidase inhibitor 16 | 1.39 | 0.2407 | 0.693321 | 1.09 |
| P69905 | Hemoglobin subunit alpha | 1.31 | 0.2553 | 0.705108 | 3.63 |
| F5GZZ9 | Scavenger receptor cysteine-rich type 1 protein | 1.29 | 0.2586 | 0.705108 | 1.20 |
| | M130 | | | | |
| P13591 | Neural cell adhesion molecule 1 | 1.27 | 0.2619 | 0.705108 | 1.03 |
| Q15113 | Procollagen C-endopeptidase enhancer 1 | 1.26 | 0.2633 | 0.705108 | 1.07 |
| P26927 | Hepatocyte growth factor-like protein | 1.24 | 0.2671 | 0.705108 | 1.03 |
| Q12841 | Follistatin-related protein 1 | 1.24 | 0.2675 | 0.705108 | 1.04 |
| Q15848 | Adiponectin | 1.24 | 0.2687 | 0.705108 | 0.95 |
| P27487 | Dipeptidyl peptidase 4 | 1.23 | 0.2706 | 0.705108 | 1.06 |
| Q86U17 | Serpin A11 | 1.22 | 0.2711 | 0.705108 | 1.11 |
| Q9UNW1 | Multiple inositol polyphosphate phosphatase 1 | 1.22 | 0.2714 | 0.705108 | 0.93 |
| P43652 | Afamin | 1.2 | 0.2758 | 0.709583 | 1.07 |
| P51884 | Lumican | 1.18 | 0.2798 | 0.712952 | 1.04 |
| P13671 | Complement component Complement | 1.15 | 0.2868 | 0.719463 | 0.99 |
| | component 6 | | | | |
| P04278 | Sex hormone-binding globulin | 1.14 | 0.2883 | 0.719463 | 0.89 |
| P03950 | Angiogenin | 1.13 | 0.2905 | 0.719463 | 0.96 |
| E9PBC5 | Plasma kallikrein | 1.1 | 0.2973 | 0.729486 | 0.96 |
| P04275 | von Willebrand factor | 1.07 | 0.303 | 0.730918 | 0.93 |
| P02748 | Complement component C9 | 1.07 | 0.3034 | 0.730918 | 0.97 |
| P08185 | Corticosteroid-binding globulin | 1.02 | 0.3139 | 0.749401 | 1.02 |
| P14625 | Endoplasmin | 1 | 0.3189 | 0.75454 | 1.05 |
| O75882 | Attractin | 0.97 | 0.3256 | 0.763575 | 1.02 |
| P68871 | Hemoglobin subunit beta | 0.96 | 0.33 | 0.767105 | 2.75 |
| P11021 | Endoplasmic reticulum chaperone BiP | 0.94 | 0.3342 | 0.770113 | 0.96 |
| P02787 | Serotransferrin | 0.9 | 0.3459 | 0.786167 | 0.97 |
| P18206 | Vinculin | 0.89 | 0.3471 | 0.786167 | 1.08 |
| Q5T7F0 | Neuropilin | 0.87 | 0.3521 | 0.790733 | 0.96 |
| P07360 | Complement component C8 gamma chain | 0.83 | 0.3655 | 0.813929 | 1.04 |
| P55058 | Phospholipid transfer protein | 0.78 | 0.3783 | 0.826519 | 1.05 |
| P01765 | Immunoglobulin heavy variable 3-23 | 0.77 | 0.3806 | 0.826519 | 0.89 |
| P13727 | Bone marrow proteoglycan | 0.74 | 0.39 | 0.826519 | 1.09 |
| P12259 | Coagulation factor V | 0.73 | 0.3962 | 0.826519 | 0.97 |
| P00742 | Coagulation factor X | 0.72 | 0.397 | 0.826519 | 1.03 |
| P18065 | Insulin-like growth factor-binding protein 2 | 0.7 | 0.4043 | 0.826519 | 0.92 |
| P54289 | Voltage-dependent calcium channel subunit | 0.7 | 0.406 | 0.826519 | 1.03 |
| | alpha-2/delta-1 | | | | |
| P13473 | Lysosome-associated membrane glycoprotein 2 | 0.7 | 0.4061 | 0.826519 | 0.92 |
| P32119 | Peroxiredoxin-2 | 0.69 | 0.4084 | 0.826519 | 1.02 |
| P04196 | Histidine-rich glycoprotein | 0.69 | 0.4087 | 0.826519 | 1.05 |
| P02649 | Apolipoprotein E | 0.69 | 0.4092 | 0.826519 | 0.96 |
| P02749 | Beta-2-glycoprotein 1 | 0.68 | 0.4107 | 0.826519 | 0.98 |
| P02655 | Apolipoprotein C-II | 0.68 | 0.4117 | 0.826519 | 1.06 |
| P00734 | Prothrombin | 0.65 | 0.4218 | 0.840429 | 0.97 |
| Q9UNN8 | Endothelial protein C receptor | 0.64 | 0.4267 | 0.843847 | 1.10 |
| P02743 | Serum amyloid P-component | 0.62 | 0.4319 | 0.847804 | 0.98 |
| P08709 | Coagulation factor VII | 0.59 | 0.4429 | 0.859138 | 1.02 |
| P02746 | Complement C1q subcomponent subunit B | 0.59 | 0.4448 | 0.859138 | 1.05 |
| P04180 | Phosphatidylcholine-sterol acyltransferase | 0.58 | 0.4474 | 0.859138 | 0.95 |
| P24821 | Tenascin | 0.55 | 0.4606 | 0.875636 | 1.05 |
| P01042 | Kininogen-1 | 0.54 | 0.4626 | 0.875636 | 0.98 |
| P23470 | Receptor-type tyrosine-protein phosphatase | 0.53 | 0.468 | 0.879133 | 1.00 |
| | gamma | | | | |
| P07195 | L-lactate dehydrogenase B chain | 0.52 | 0.4712 | 0.879133 | 0.95 |
| Q13822 | Ectonucleotide pyrophosphatase/ | 0.51 | 0.4744 | 0.879133 | 1.02 |
| | phosphodiesterase family member 2 | | | | |
| P49908 | Selenoprotein P | 0.5 | 0.481 | 0.885174 | 1.00 |
| P04114 | Apolipoprotein B-100 | 0.49 | 0.4845 | 0.885466 | 0.99 |
| P02766 | Transthyretin | 0.47 | 0.4941 | 0.890905 | 0.95 |
| P27918 | Properdin | 0.47 | 0.4942 | 0.890905 | 1.05 |
| H0Y897 | Target of Nesh-SH3 | 0.44 | 0.5101 | 0.902403 | 1.06 |
| P01344 | Insulin-like growth factor II | 0.44 | 0.5104 | 0.902403 | 0.97 |
| P01031 | Complement C5 | 0.42 | 0.5163 | 0.902403 | 1.01 |
| P02776 | Platelet factor 4 | 0.41 | 0.5214 | 0.902403 | 0.94 |
| P06727 | Apolipoprotein A-IV | 0.41 | 0.5222 | 0.902403 | 1.01 |
| P05155 | Plasma protease C1 inhibitor | 0.41 | 0.5226 | 0.902403 | 0.96 |
| P15169 | Carboxypeptidase N catalytic chain | 0.41 | 0.5251 | 0.902403 | 0.97 |
| Q9BXR6 | Complement factor H-related protein 5 | 0.39 | 0.5319 | 0.902403 | 1.00 |
| P02656 | Apolipoprotein C-III | 0.39 | 0.5337 | 0.902403 | 0.95 |
| Q16270 | Insulin-like growth factor-binding protein 7 | 0.39 | 0.5361 | 0.902403 | 0.96 |
| 095445 | Apolipoprotein M | 0.37 | 0.5426 | 0.902403 | 0.98 |
| P00488 | Coagulation factor XIII A chain | 0.37 | 0.5441 | 0.902403 | 1.01 |
| Q76LX8 | A disintegrin and metalloproteinase with | 0.36 | 0.5492 | 0.902403 | 0.97 |
| | thrombospondin motifs 13 | | | | |
| Q8IUL8 | Cartilage intermediate layer protein 2 | 0.35 | 0.5553 | 0.902403 | 1.07 |
| P61769 | Beta-2-micro globulin | 0.34 | 0.5628 | 0.902403 | 1.05 |
| Q99983 | Osteomodulin | 0.33 | 0.5661 | 0.902403 | 1.04 |
| P54802 | Alpha-N-acetylglucosaminidase | 0.32 | 0.5723 | 0.902403 | 1.11 |
| P11226 | Mannose-binding protein C | 0.32 | 0.5724 | 0.902403 | 0.98 |
| P05543 | Thyroxine-binding globulin | 0.32 | 0.5731 | 0.902403 | 0.99 |
| P10721 | Mast/stem cell growth factor receptor Kit | 0.32 | 0.5743 | 0.902403 | 1.01 |
| Q14520 | Hyaluronan-binding protein 2 | 0.31 | 0.5759 | 0.902403 | 0.98 |
| P01861 | Immunoglobulin heavy constant gamma 4 | 0.31 | 0.5783 | 0.902403 | 0.82 |
| P00748 | Coagulation factor XII | 0.31 | 0.5789 | 0.902403 | 0.98 |
| P09172 | Dopamine beta-hydroxylase | 0.29 | 0.5883 | 0.908087 | 0.97 |
| Q16610 | Extracellular matrix protein 1 | 0.29 | 0.5894 | 0.908087 | 0.98 |
| P36980 | Complement factor H-related protein 2 | 0.28 | 0.5952 | 0.90842 | 0.91 |
| Q9UHG3 | Prenylcysteine oxidase 1 | 0.28 | 0.5982 | 0.90842 | 1.02 |
| P01008 | Antithrombin-III | 0.28 | 0.5999 | 0.90842 | 0.98 |
| P01009 | Alpha-1-antitrypsin | 0.24 | 0.6231 | 0.935734 | 0.99 |
| P33151 | Cadherin-5 | 0.24 | 0.625 | 0.935734 | 0.96 |
| P17936 | Insulin-like growth factor-binding protein 3 | 0.23 | 0.6329 | 0.941564 | 1.06 |
| P01876 | Immunoglobulin heavy constant alpha 1 | 0.23 | 0.636 | 0.941564 | 0.96 |
| P22352 | Glutathione peroxidase 3 | 0.22 | 0.6413 | 0.943898 | 1.00 |
| P01034 | Cystatin-C | 0.21 | 0.6447 | 0.943898 | 1.03 |
| P28827 | Receptor-type tyrosine-protein phosphatase mu | 0.2 | 0.6555 | 0.95038 | 0.98 |
| P36955 | Pigment epithelium-derived factor | 0.2 | 0.6563 | 0.95038 | 1.00 |
| P59666 | Neutrophil defensin 3 | 0.19 | 0.6605 | 0.951264 | 0.99 |
| P18428 | Lipopolysaccharide-binding protein | 0.18 | 0.6698 | 0.959443 | 1.05 |
| P43121 | Cell surface glycoprotein MUC18 | 0.17 | 0.6784 | 0.966538 | 0.98 |
| P19652 | Alpha-1-acid glycoprotein 2 | 0.15 | 0.7034 | 0.979948 | 1.13 |
| P04075 | Fructose-bisphosphate aldolase A | 0.14 | 0.7057 | 0.979948 | 0.97 |
| Q6EMK4 | Vasorin | 0.14 | 0.7064 | 0.979948 | 0.99 |
| Q14515 | SPARC-like protein 1 | 0.14 | 0.7085 | 0.979948 | 1.05 |
| P05109 | Protein S100-A8 | 0.14 | 0.7099 | 0.979948 | 1.01 |
| P05546 | Heparin cofactor 2 | 0.14 | 0.71 | 0.979948 | 1.03 |
| P49747 | Cartilage oligomeric matrix protein | 0.13 | 0.7175 | 0.981183 | 1.00 |
| Q9NPY3 | Complement component C1q receptor | 0.13 | 0.7183 | 0.981183 | 1.01 |
| Q9Y4L1 | Hypoxia up-regulated protein 1 | 0.12 | 0.7318 | 0.992682 | 1.02 |
| P02774 | Vitamin D-binding protein | 0.11 | 0.746 | 0.992682 | 0.97 |
| P00736 | Complement C1r subcomponent | 0.1 | 0.7467 | 0.992682 | 0.99 |
| P19827 | Inter-alpha-trypsin inhibitor heavy chain H1 | 0.1 | 0.749 | 0.992682 | 0.98 |
| P06702 | Protein S100-A9 | 0.1 | 0.7506 | 0.992682 | 1.00 |
| O00391 | Sulfhydryl oxidase 1 | 0.1 | 0.7547 | 0.992682 | 1.01 |
| Q92820 | Gamma-glutamyl hydrolase | 0.09 | 0.759 | 0.992682 | 1.03 |
| P80108 | Phosphatidylinositol-glycan-specific | 0.09 | 0.7649 | 0.992682 | 1.01 |
| | phospholipase D | | | | |
| Q9NPH3 | Interleukin-1 receptor accessory protein | 0.09 | 0.7649 | 0.992682 | 1.06 |
| P01011 | Alpha-1-antichymotrypsin | 0.08 | 0.7712 | 0.992682 | 0.98 |
| P35542 | Serum amyloid A-4 protein | 0.08 | 0.7743 | 0.992682 | 1.03 |
| P02763 | Alpha-1-acid glycoprotein 1 | 0.08 | 0.7752 | 0.992682 | 0.90 |
| P20742 | Pregnancy zone protein | 0.08 | 0.7787 | 0.992682 | 1.05 |
| P02775 | Platelet basic protein | 0.08 | 0.7815 | 0.992682 | 0.99 |
| O95479 | GDH/6PGL endoplasmic bifunctional protein | 0.08 | 0.7835 | 0.992682 | 0.99 |
| P05362 | Intercellular adhesion molecule 1 | 0.07 | 0.7875 | 0.992682 | 0.95 |
| O14791 | Apolipoprotein L1 | 0.07 | 0.7904 | 0.992682 | 1.06 |
| P33908 | Mannosyl-oligosaccharide 1,2-alpha- | 0.07 | 0.7947 | 0.993375 | 0.98 |
| | mannosidase IA | | | | |
| P22891 | Vitamin K-dependent protein Z | 0.06 | 0.801 | 0.993379 | 0.91 |
| Q13740 | CD166 antigen | 0.06 | 0.8022 | 0.993379 | 0.97 |
| P22105 | Tenascin-X | 0.05 | 0.816 | 0.9976 | 1.00 |
| P05156 | Complement factor I | 0.05 | 0.8205 | 0.9976 | 1.01 |
| Q96KN2 | Beta-Ala-His dipeptidase | 0.05 | 0.8245 | 0.9976 | 1.03 |
| P10643 | Complement component C7 | 0.04 | 0.8343 | 0.9976 | 1.00 |
| O00533 | Neural cell adhesion molecule L1-like protein | 0.04 | 0.8359 | 0.9976 | 0.97 |
| Q9BWP8 | Collectin-11 | 0.04 | 0.8409 | 0.9976 | 0.98 |
| Q99969 | Retinoic acid receptor responder protein 2 | 0.04 | 0.8425 | 0.9976 | 0.99 |
| P04070 | Vitamin K-dependent protein C | 0.04 | 0.8443 | 0.9976 | 1.02 |
| P04066 | Tissue alpha-L-fucosidase | 0.04 | 0.8513 | 0.9976 | 0.94 |
| P55056 | Apolipoprotein C-IV | 0.03 | 0.8585 | 0.9976 | 1.12 |
| P0C0L5 | Complement C4-B | 0.03 | 0.8646 | 0.9976 | 0.99 |
| P00450 | Ceruloplasmin | 0.03 | 0.8653 | 0.9976 | 0.99 |
| P02760 | Protein AMBP | 0.03 | 0.8721 | 0.9976 | 1.00 |
| P19320 | Vascular cell adhesion protein 1 | 0.02 | 0.884 | 0.9976 | 0.98 |
| P22692 | Insulin-like growth factor-binding protein 4 | 0.02 | 0.8848 | 0.9976 | 0.59 |
| P02751 | Fibronectin | 0.02 | 0.8998 | 0.9976 | 0.97 |
| P08519 | Apolipoprotein | 0.01 | 0.9049 | 0.9976 | 1.20 |
| P12830 | Cadherin-1 | 0.01 | 0.9146 | 0.9976 | 0.94 |
| Q96IY4 | Carboxypeptidase B2 | 0.01 | 0.919 | 0.9976 | 1.01 |
| P17813 | Endoglin | 0.01 | 0.9194 | 0.9976 | 0.97 |
| P35443 | Thrombospondin-4 | 0.01 | 0.9285 | 0.9976 | 1.11 |
| P98160 | Basement membrane-specific heparan sulfate | 0.01 | 0.9306 | 0.9976 | 0.98 |
| | proteoglycan core protein | | | | |
| P00738 | Haptoglobin | 0.01 | 0.9311 | 0.9976 | 0.98 |
| P19823 | Inter-alpha-trypsin inhibitor heavy chain H2 | 0.01 | 0.9331 | 0.9976 | 1.00 |
| P02790 | Hemopexin | 0.01 | 0.9342 | 0.9976 | 1.00 |
| Q06033 | Inter-alpha-trypsin inhibitor heavy chain H3 | 0.01 | 0.9358 | 0.9976 | 0.97 |
| Q07954 | Prolow-density lipoprotein receptor-related | 0.01 | 0.9388 | 0.9976 | 0.98 |
| | protein 1 | | | | |
| P0C0L4 | Complement C4-A | 0.01 | 0.939 | 0.9976 | 1.03 |
| P00740 | Coagulation factor IX | 0 | 0.9441 | 0.9976 | 1.01 |
| P07333 | Macrophage colony-stimulating factor 1 | 0 | 0.9442 | 0.9976 | 0.98 |
| | receptor | | | | |
| Q92954 | Proteoglycan 4 | 0 | 0.9448 | 0.9976 | 1.01 |
| Q6YHK3 | CD109 antigen | 0 | 0.9453 | 0.9976 | 0.97 |
| Q99784 | Noelin | 0 | 0.949 | 0.9976 | 1.00 |
| Q01459 | Di-N-acetylchitobiase | 0 | 0.9502 | 0.9976 | 0.99 |
| Q9Y6R7 | IgGFc-binding protein | 0 | 0.9507 | 0.9976 | 1.06 |
| Q9NZP8 | Complement C1r subcomponent-like protein | 0 | 0.9535 | 0.9976 | 1.01 |
| P04217 | Alpha-1B-glycoprotein | 0 | 0.955 | 0.9976 | 1.00 |
| K7EMN2 | 6-phosphogluconate dehydrogenase, | 0 | 0.959 | 0.9976 | 0.95 |
| | decarboxylating | | | | |
| G3V2W1 | Protein Z-dependent protease inhibitor | 0 | 0.9607 | 0.9976 | 1.00 |
| P12955 | Xaa-Pro dipeptidase | 0 | 0.961 | 0.9976 | 0.94 |
| P06681 | Complement C2 | 0 | 0.9688 | 0.9976 | 1.02 |
| B7ZKJ8 | ITIH4 protein | 0 | 0.9701 | 0.9976 | 0.99 |
| P05154 | Plasma serine protease inhibitor | 0 | 0.9707 | 0.9976 | 1.00 |
| P13796 | Plastin-2 | 0 | 0.9799 | 0.9976 | 0.98 |
| P09486 | SPARC | 0 | 0.9809 | 0.9976 | 0.95 |
| P02765 | Alpha-2-HS-glycoprotein | 0 | 0.9812 | 0.9976 | 1.01 |
| P05160 | Coagulation factor XIII B chain | 0 | 0.9826 | 0.9976 | 1.00 |
| P54108 | Cysteine-rich secretory protein 3 | 0 | 0.9883 | 0.9976 | 0.98 |
| Q16853 | Membrane primary amine oxidase | 0 | 0.9943 | 0.9976 | 0.98 |
| P05090 | Apolipoprotein D | 0 | 0.9949 | 0.9976 | 0.92 |
| Q10588 | ADP-ribosyl cyclase/cyclic ADP-ribose | 0 | 0.9976 | 0.9976 | 0.97 |
| | hydrolase 2 | | | | |

PEs: psychotic experiences; FDR: false discovery rate; ALSPAC: Avon Longitudinal Study of Parents and Children

TABLE 15
Fold changes in poor functioning vs. good functioning

| | | | | Ratio |
|---|---|---|---|---|
| | | | | of |
| | | | | means |
| | | Mean LFQ, | Mean LFQ, | (poor |
| Uniprot | | poor | good | vs |
| No. | Protein name | functioning | functioning | good) |
| P63104 | 14-3-3 protein zeta/delta | 27324340 | 37927971 | 0.72 |
| Q76LX8 | A disintegrin and metalloproteinase | 10308467 | 11282881 | 0.91 |
| | with thrombospondin motifs 13 | | | |
| P60709 | Actin, cytoplasmic 1 | 2.91E+08 | 3.96E+08 | 0.73 |
| P43652 | Afamin | 1.86E+09 | 1.71E+09 | 1.09 |
| P02763 | Alpha-1-acid glycoprotein 1 | 1.66E+08 | 1.86E+08 | 0.89 |
| P01011 | Alpha-1-antichymotrypsin | 5.35E+09 | 5.3E+09 | 1.01 |
| P01009 | Alpha-1-antitrypsin | 2.63E+08 | 3.19E+08 | 0.83 |
| P04217 | Alpha-1B-glycoprotein | 3.68E+09 | 3.46E+09 | 1.06 |
| P08697 | Alpha-2-antiplasmin | 1.18E+09 | 1.17E+09 | 1.01 |
| P02765 | Alpha-2-HS-glycoprotein | 1.45E+09 | 1.37E+09 | 1.06 |
| P01023 | Alpha-2-macroglobulin | 1.74E+09 | 3.47E+09 | 0.50 |
| P12814 | Alpha-actinin-1 | 34823635 | 52957479 | 0.66 |
| P02489 | Alpha-crystallin A chain | 1.46E+08 | 1.26E+08 | 1.15 |
| P15144 | Aminopeptidase N | 13227156 | 12992373 | 1.02 |
| P01019 | Angiotensinogen | 3.93E+09 | 3.46E+09 | 1.14 |
| P01008 | Antithrombin-III | 5.28E+09 | 4.9E+09 | 1.08 |
| P02647 | Apolipoprotein A-I | 9.24E+08 | 1.18E+09 | 0.78 |
| P02652 | Apolipoprotein A-II | 3.14E+08 | 4.2E+08 | 0.75 |
| P06727 | Apolipoprotein A-IV | 4.79E+09 | 4.46E+09 | 1.07 |
| P04114 | Apolipoprotein B-100 | 2.04E+10 | 2E+10 | 1.02 |
| P02654 | Apolipoprotein C-I | 1.65E+08 | 1.69E+08 | 0.97 |
| P02656 | Apolipoprotein C-III | 3.97E+08 | 2.74E+08 | 1.45 |
| P05090 | Apolipoprotein D | 31119194 | 28321812 | 1.10 |
| P02649 | Apolipoprotein E | 7.21E+08 | 7.01E+08 | 1.03 |
| O75882 | Attractin | 1.02E+08 | 86797031 | 1.18 |
| P02749 | Beta-2-glycoprotein 1 | 5.14E+09 | 4.43E+09 | 1.16 |
| Q96KN2 | Beta-Ala-His dipeptidase | 1E+08 | 1E+08 | 1.00 |
| P43320 | Beta-crystallin B2 | 93275660 | 75149812 | 1.24 |
| P43251 | Biotinidase | 1.46E+08 | 1.37E+08 | 1.07 |
| P04003 | C4b-binding protein alpha chain | 7.05E+08 | 7.41E+08 | 0.95 |
| P00915 | Carbonic anhydrase 1 | 55227827 | 65693374 | 0.84 |
| Q96IY4 | Carboxypeptidase B2 | 1.37E+08 | 1.2E+08 | 1.14 |
| P15169 | Carboxypeptidase N catalytic chain | 1.43E+08 | 1.41E+08 | 1.02 |
| P22792 | Carboxypeptidase N subunit 2 | 2.47E+08 | 2.47E+08 | 1.00 |
| P49747 | Cartilage oligomeric matrix protein | 20260311 | 19701309 | 1.03 |
| O43866 | CD5 antigen-like | 39540069 | 42070317 | 0.94 |
| P00450 | Ceruloplasmin | 5.54E+09 | 4.79E+09 | 1.16 |
| P06276 | Cholinesterase | 1.33E+08 | 1.17E+08 | 1.13 |
| P10909 | Clusterin | 1.5E+09 | 1.22E+09 | 1.22 |
| P00740 | Coagulation factor IX | 1.48E+08 | 1.28E+08 | 1.16 |
| P12259 | Coagulation factor V | 3.65E+08 | 3.55E+08 | 1.03 |
| P00742 | Coagulation factor X | 1.65E+08 | 1.44E+08 | 1.15 |
| P03951 | Coagulation factor XI | 36617287 | 32359907 | 1.13 |
| P00748 | Coagulation factor XII | 5.97E+08 | 5.25E+08 | 1.14 |
| P00488 | Coagulation factor XIII A chain | 1.86E+08 | 2.08E+08 | 0.89 |
| P05160 | Coagulation factor XIII B chain | 1.02E+08 | 87470935 | 1.16 |
| P23528 | Cofilin-1 | 33766862 | 46939930 | 0.72 |
| P02746 | Complement C1q subcomponent | 6.61E+08 | 6.82E+08 | 0.97 |
| | subunit B | | | |
| P02747 | Complement C1q subcomponent | 1.73E+08 | 1.37E+08 | 1.26 |
| | subunit C | | | |
| P00736 | Complement C1r subcomponent | 8.71E+08 | 7.53E+08 | 1.16 |
| Q9NZP8 | Complement C1r subcomponent-like | 71274570 | 66217400 | 1.08 |
| | protein | | | |
| P09871 | Complement C1s subcomponent | 7.53E+08 | 7.07E+08 | 1.07 |
| P06681 | Complement C2 | 2.8E+08 | 2.87E+08 | 0.98 |
| P01024 | Complement C3 | 4.07E+08 | 4.76E+08 | 0.85 |
| P0C0L4 | Complement C4-A | 1.71E+10 | 1.6E+10 | 1.07 |
| P0C0L5 | Complement C4-B | 3.54E+08 | 3.09E+08 | 1.14 |
| P01031 | Complement C5 | 1.88E+09 | 1.74E+09 | 1.08 |
| P13671 | Complement component C6 | 8.14E+08 | 7.85E+08 | 1.04 |
| P10643 | Complement component C7 | 5.17E+08 | 4.59E+08 | 1.12 |
| P07357 | Complement component C8 alpha chain | 2.12E+08 | 1.77E+08 | 1.20 |
| P07358 | Complement component C8 beta chain | 3.8E+08 | 3.84E+08 | 0.99 |
| P07360 | Complement component C8 gamma | 3.21E+08 | 3.22E+08 | 1.00 |
| | chain | | | |
| P02748 | Complement component C9 | 1.29E+09 | 1.26E+09 | 1.02 |
| P00751 | Complement factor B | 4.24E+09 | 3.93E+09 | 1.08 |
| P08603 | Complement factor H | 4.18E+09 | 3.61E+09 | 1.16 |
| Q03591 | Complement factor H-related protein 1 | 80560638 | 67834507 | 1.19 |
| P36980 | Complement factor H-related protein 2 | 80956562 | 69473412 | 1.17 |
| Q9BXR6 | Complement factor H-related protein 5 | 26041456 | 20054874 | 1.30 |
| P05156 | Complement factor I | 1.33E+09 | 1.11E+09 | 1.20 |
| P08185 | Corticosteroid-binding globulin | 2.33E+08 | 2.27E+08 | 1.02 |
| P09172 | Dopamine beta-hydroxylase | 29293024 | 25464467 | 1.15 |
| P11021 | Endoplasmic reticulum chaperone BiP | 10076120 | 11107986 | 0.91 |
| Q16610 | Extracellular matrix protein 1 | 2.67E+08 | 2.48E+08 | 1.08 |
| Q86UX7 | Fermitin family homolog 3 | 75769339 | 1.13E+08 | 0.67 |
| Q9UGM5 | Fetuin-B | 1.96E+08 | 1.35E+08 | 1.45 |
| P02671 | Fibrinogen alpha chain | 4.95E+08 | 5.38E+08 | 0.92 |
| P02675 | Fibrinogen beta chain | 5.56E+08 | 5.27E+08 | 1.05 |
| P02679 | Fibrinogen gamma chain | 4.2E+08 | 4.52E+08 | 0.93 |
| P02751 | Fibronectin | 6.03E+09 | 5.25E+09 | 1.15 |
| P23142 | Fibulin-1 | 70803799 | 59337625 | 1.19 |
| O75636 | Ficolin-3 | 2.01E+08 | 1.83E+08 | 1.10 |
| P21333 | Filamin-A | 37834622 | 61900410 | 0.61 |
| P04075 | Fructose-bisphosphate aldolase A | 35345594 | 52888573 | 0.67 |
| Q08380 | Galectin-3-binding protein | 12756921 | 10979333 | 1.16 |
| P06396 | Gelsolin | 1.83E+09 | 1.65E+09 | 1.11 |
| P22352 | Glutathione peroxidase 3 | 1.11E+08 | 1.24E+08 | 0.89 |
| P00738 | Haptoglobin | 5E+08 | 5.68E+08 | 0.88 |
| P68871 | Hemoglobin subunit beta | 1.89E+08 | 1.45E+08 | 1.30 |
| P02790 | Hemopexin | 1.02E+10 | 9.65E+09 | 1.06 |
| P05546 | Heparin cofactor 2 | 2.37E+09 | 2.23E+09 | 1.06 |
| Q04756 | Hepatocyte growth factor activator | 95828468 | 93649078 | 1.02 |
| P26927 | Hepatocyte growth factor-like protein | 1.3E+08 | 1.15E+08 | 1.13 |
| P04196 | Histidine-rich glycoprotein | 1.57E+09 | 1.3E+09 | 1.21 |
| Q14520 | Hyaluronan-binding protein 2 | 1.24E+08 | 1.06E+08 | 1.17 |
| Q9Y6R7 | IgGFc-binding protein | 44637880 | 40960162 | 1.09 |
| P01876 | Immunoglobulin heavy constant alpha 1 | 1.69E+08 | 1.89E+08 | 0.89 |
| P01857 | Immunoglobulin heavy constant gamma | 1.47E+08 | 2E+08 | 0.74 |
| | 1 | | | |
| P01859 | Immunoglobulin heavy constant gamma | 62720442 | 80586495 | 0.78 |
| | 2 | | | |
| P01860 | Immunoglobulin heavy constant gamma | 5.72E+08 | 8.1E+08 | 0.71 |
| | 3 | | | |
| P01871 | Immunoglobulin heavy constant mu | 1.64E+08 | 2.88E+08 | 0.57 |
| P01834 | Immunoglobulin kappa constant | 1.15E+08 | 1.54E+08 | 0.75 |
| P01623 | Immunoglobulin kappa variable 3-20 | 23132218 | 31380021 | 0.74 |
| P0CG06 | Immunoglobulin lambda constant 2 | 1.1E+08 | 1.89E+08 | 0.58 |
| P17936 | Insulin-like growth factor-binding | 1.48E+08 | 1.26E+08 | 1.17 |
| | protein 3 | | | |
| P35858 | Insulin-like growth factor-binding | 7.02E+08 | 6.49E+08 | 1.08 |
| | protein complex acid labile subunit | | | |
| P19827 | Inter-alpha-trypsin inhibitor heavy chain | 2.87E+09 | 2.29E+09 | 1.25 |
| | H1 | | | |
| P19823 | Inter-alpha-trypsin inhibitor heavy chain | 3.27E+09 | 2.97E+09 | 1.10 |
| | H2 | | | |
| Q06033 | Inter-alpha-trypsin inhibitor heavy chain | 2.2E+08 | 2.45E+08 | 0.90 |
| | H3 | | | |
| Q14624 | Inter-alpha-trypsin inhibitor heavy chain | 4.48E+09 | 4.33E+09 | 1.03 |
| | H4 | | | |
| Q9NPH3 | Interleukin-1 receptor accessory protein | 20168003 | 23175142 | 0.87 |
| P29622 | Kallistatin | 7.05E+08 | 6.29E+08 | 1.12 |
| P01042 | Kininogen-1 | 4.4E+09 | 3.63E+09 | 1.21 |
| P02750 | Leucine-rich alpha-2-glycoprotein | 7.31E+08 | 7.76E+08 | 0.94 |
| P18428 | Lipopolysaccharide-binding protein | 1.38E+08 | 1.35E+08 | 1.02 |
| P00338 | L-lactate dehydrogenase A chain | 8952806 | 11233067 | 0.80 |
| P07195 | L-lactate dehydrogenase B chain | 26817779 | 29679402 | 0.90 |
| P51884 | Lumican | 5.61E+08 | 4.64E+08 | 1.21 |
| P48740 | Mannan-binding lectin serine protease 1 | 32715093 | 31407783 | 1.04 |
| P11226 | Mannose-binding protein C | 53244859 | 42113257 | 1.26 |
| P08571 | Monocyte differentiation antigen CD14 | 1.28E+08 | 1.29E+08 | 1.00 |
| Q96PD5 | N-acetylmuramoyl-L-alanine amidase | 5.66E+08 | 5.24E+08 | 1.08 |
| O00533 | Neural cell adhesion molecule L1-like | 11603131 | 10077883 | 1.15 |
| | protein | | | |
| P30041 | Peroxiredoxin-6 | 16785662 | 21645949 | 0.78 |
| P80108 | Phosphatidy linositol-glycan-specific | 2.45E+08 | 2.07E+08 | 1.18 |
| | phospholipase D | | | |
| P55058 | Phospholipid transfer protein | 8536523 | 12589594 | 0.68 |
| P36955 | Pigment epithelium-derived factor | 9.44E+08 | 8.2E+08 | 1.15 |
| P03952 | Plasma kallikrein | 5.52E+08 | 4.87E+08 | 1.13 |
| P05155 | Plasma protease C1 inhibitor | 1.77E+09 | 1.82E+09 | 0.97 |
| P05154 | Plasma serine protease inhibitor | 1.68E+08 | 1.55E+08 | 1.09 |
| P00747 | Plasminogen | 3.82E+09 | 3.11E+09 | 1.23 |
| P13796 | Plastin-2 | 35697546 | 34051982 | 1.05 |
| P02775 | Platelet basic protein | 1.92E+08 | 3.07E+08 | 0.63 |
| P07359 | Platelet glycoprotein Ib alpha chain | 58568238 | 61100167 | 0.96 |
| P20742 | Pregnancy zone protein | 1.62E+08 | 47918310 | 3.39 |
| P07737 | Profilin-1 | 44916658 | 69435692 | 0.65 |
| P02760 | Protein AMBP | 1.23E+09 | 1.2E+09 | 1.02 |
| Q9UK55 | Protein Z-dependent protease inhibitor | 71636100 | 63032835 | 1.14 |
| Q92954 | Proteoglycan 4 | 46278448 | 45413513 | 1.02 |
| P00734 | Prothrombin | 4.6E+09 | 4.17E+09 | 1.10 |
| P14618 | Pyruvate kinase PKM | 19533899 | 33469384 | 0.58 |
| Q12913 | Receptor-type tyrosine-protein | 8648297 | 8875812 | 0.97 |
| | phosphatase eta | | | |
| P02753 | Retinol-binding protein 4 | 6.12E+08 | 5.07E+08 | 1.21 |
| P49908 | Selenoprotein P | 89654111 | 94473461 | 0.95 |
| P02787 | Serotransferrin | 7.14E+08 | 7.72E+08 | 0.92 |
| P02743 | Serum amyloid P-component | 7.99E+08 | 7.47E+08 | 1.07 |
| P27169 | Serum paraoxonase/arylesterase 1 | 72238374 | 66744222 | 1.08 |
| P04278 | Sex hormone-binding globulin | 1.26E+08 | 94259609 | 1.33 |
| O00391 | Sulfhydryl oxidase 1 | 59043964 | 58513437 | 1.01 |
| Q9Y490 | Talin-1 | 1.33E+08 | 2.24E+08 | 0.60 |
| P22105 | Tenascin-X | 30161188 | 28254344 | 1.07 |
| P05452 | Tetranectin | 4.82E+08 | 4.58E+08 | 1.05 |
| P07996 | Thrombospondin-1 | 92902684 | 1.24E+08 | 0.75 |
| P05543 | Thyroxine-binding globulin | 1.9E+08 | 1.59E+08 | 1.19 |
| Q15582 | Transforming growth factor-beta- | 25159916 | 22518702 | 1.12 |
| | induced protein ig-h3 | | | |
| P02766 | Transthyretin | 26501841 | 42941787 | 0.62 |
| P60174 | Triosephosphate isomerase | 8978713 | 12621377 | 0.71 |
| P19320 | Vascular cell adhesion protein 1 | 19702232 | 19258760 | 1.20 |
| Q6EMK4 | Vasorin | 23990562 | 25021406 | 0.96 |
| P18206 | Vinculin | 66442583 | 1.1E+08 | 0.60 |
| P02774 | Vitamin D-binding protein | 6.68E+09 | 5.45E+09 | 1.23 |
| P04070 | Vitamin K-dependent protein C | 48342064 | 42493971 | 1.14 |
| P07225 | Vitamin K-dependent protein S | 2.41E+08 | 2.43E+08 | 0.99 |
| P22891 | Vitamin K-dependent protein Z | 32900507 | 29571834 | 1.11 |
| P04004 | Vitronectin | 3.44E+09 | 3.18E+09 | 1.08 |
| P04275 | von Willebrand factor | 91148073 | 85190545 | 1.07 |
| P25311 | Zinc-alpha-2-glycoprotein | 1.71E+09 | 1.74E+09 | 0.98 |

### Example 2

Another set of models were developed without incorporating correction for covariates. Here, instead, the previous covariates (age, sex, BMI and years in education) were included as clinical features in and of themselves in the models. Methods and results are explained in detail below.

### Methods

As above, we used Neurominer v.1.0 (https://www.pronia.eu/neurominer/) for MatLab 2018a (MathWorks Inc.) to develop support vector machine (SVM) models. For all models, hyper-parameters were optimised in repeated nested cross-validation (see eMethods) and area under the receiver-operating characteristic curve (AUC) was the performance evaluation criterion. Random-label permutation analysis with 1000 permutations was used to verify against a null distribution and derive p-values for statistical significance. Missing clinical data were replaced using the mean (for continuous) or modal value (for categorical variables). Continuous clinical variables were converted to z-scores and winsorised within ±3z.

### Models 1a-c: Predicting Transition Using Clinical and Proteomic Data

First, we developed a model predicting transition outcome based on the clinical and proteomic data in combination (Model 1a). The included clinical features are listed in Table 16 below. We incorporated geographical generalisability by using a leave-site-out cross-validation approach (see eMethods) as recommended for data from multisite consortia. We used the LIBLINEAR program with L2 regularisation to attenuate over-fitting whereby weightings of non-predictive features are minimised, but not reduced to zero (thus more closely modelling the biological effects of functionally inter-related proteins). The hyperplane was weighted (increasing the misclassification penalty in the minority class) which reduces the risk of bias in unbalanced group sizes.

Next, to assess the relative contribution of clinical and proteomic data, we developed models using the same cross-validation and training framework but based on clinical features (Model 1b) and proteomic features (Model 1c) separately.

### Models 2a-b: Parsimonious Model

We sought to generate a parsimonious model based on the 10 highest-weighted proteomic predictors from a London test site, and the model was validated.

To derive the 10 highest-weighted proteins, we generated an L2-regularised SVM model (Model 2a) using proteomic data from all sites except London (CHR-T n=30, CHR-NT n=50). A reduced model was then developed based solely on data for these 10 proteins in the non-London dataset (Model 2b), before being tested in the London data (CHR-T n=19, CHR-NT n=34). Both models used leave-site-out cross-validation.

### Model 3: Replication

Due to differences in protein identifications, it was not possible to apply the above models in the replication dataset. We instead sought to replicate our initial findings by conducting a second discovery analysis, generating a new L2-regularised SVM model (with leave-site-out cross-validation) for prediction of transition status based on the clinical and proteomic data in the replication dataset.

### Results

### Model 1a: Predicting Transition Using Clinical and Proteomic Data

An SVM model predicted transition status based on 65 clinical and 166 proteomic features (Model 1a) with excellent performance (AUC 0.95, p<0.001). Performance metrics are presented in Table 17. FIG. 11a shows mean algorithm scores and predicted outcomes stratified by site. The receiver-operating characteristic curve is shown in FIG. 11b. Table 18 lists the 10% highest-weighted features based on mean feature weighting across all models selected in the inner loop.

### Model 1b: Clinical Data

The clinical model (Model 11b) demonstrated poor predictive performance (AUC 0.48, p=0.628; Table 17, FIG. 12).

### Model 1c: Proteomic Data

The proteomic model (Model 1c) demonstrated excellent predictive performance (AUC 0.96, p<0.001; Table 17, FIG. 13).

### Models 2a-b: Parsimonious Model

The AUC for the model based on proteomic data from all sites except London (Model 2a) was 0.94, p<0.001 (Table 17, FIG. 14). The 10 highest-weighted features were: alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6).

A reduced model based solely on these 10 features was then trained using data from all sites except London (Model 2b) with AUC 0.99, p<0.001 (Table 17, FIG. 15). This model predicted transition status in the London data (CHR-T n=19, CHR-NT n=34) with AUC 0.92 (Table 17).

### Model 3: Replication

This model demonstrated excellent performance for prediction of transition outcome in the replication dataset (AUC 0.98, p<0.001; Table 17, FIG. 16). The highest-weighted 10% of features are shown in Table 18 Proteins among the highest-weighted 10% of features in both Model 1a and Model 3 (and weighted in similar directions) included: A2M, IGHM, C4BPA, plasminogen and complement component 6 (C6).

TABLE 16
List of 69 baseline EU-GEI clinical features included
in Model 1a, Model 1b and Model 3

Age
Sex
Body mass index
Years in education
GAF symptoms
GAF disability
SANS: unchanging facial expression
SANS: decreased spontaneous movements
SANS: paucity of expressive gestures
SANS: poor eye contact
SANS: affective nonresponsivity
SANS: inappropriate affect
SANS: lack of vocal inflections
SANS: global rating of affective flattening
SANS: poverty of speech
SANS: poverty of speech content
SANS: blocking
SANS: increased latency of response
SANS: global rating of alogia
SANS: grooming and hygiene
SANS: impersistence at work or school
SANS: physical anergia
SANS: global rating for avolition-apathy
SANS: recreational interests and activities
SANS: sexual activity
SANS: ability to feel intimacy and closeness
SANS: relationship with friends and peers
SANS: global rating of anhedonia-asociality
SANS: social inattentiveness
SANS: inattentiveness during mental status testing
SANS: global rating of attention
Total SANS composite score
Total SANS global score
BPRS: somatic concern
BPRS: anxiety
BPRS: depression
BPRS: suicidality
BPRS: guilt
BPRS: hostility
BPRS: elevated mood
BPRS: grandiosity
BORS: suspiciousness
BPRS: hallucinations
BPRS: unusual thought content
BPRS: bizarre behaviour
BPRS: self-neglect
BPRS: disorientation
BPRS: conceptual disorganisation
BPRS: blunted affect
BPRS: emotional withdrawal
BPRS: motor retardation
BPRS: tension
BPRS: uncooperativeness
BPRS: excitement
BPRS: distractibility
BPRS: motor hyperactivity
BPRS: mannerisms and posturing
Total BPRS score
MADRS: apparent sadness
MADRS: reported sadness
MADRS: inner tension
MADRS: reduced sleep
MADRS: reduced appetite
MADRS: concentration difficulties
MADRS: lassitude
MADRS: inability to feel
MADRS: pessimistic thoughts
MADRS: suicidal thoughts
Total MADRS score
GAF: General Assessment of Functioning; SANS: Scale for the Assessment of Negative Symptoms; BPRS: Brief Psychiatric Rating Scale; MADRS: Montgomery Asberg Depression Rating Scale; EU-GEI: European Network of National Schizophrenia Networks Studying Gene-Environment Interactions.

TABLE 17
Performance metrics for unadjusted support vector machine models

| | | | | | | | Bal- | | | | | |
|---|---|---|---|---|---|---|---|---|---|---|---|---|
| | True | False | True | False | | | anced | AUC | Positive | Negative | Positive | Negative |
| | posi- | nega- | nega- | posi- | Sensi- | Speci- | accu- | (95% | predic- | predic- | likeli- | likeli- |
| | tives, | tives, | tives, | tives, | tivity, | ficity, | racy, | confidence | tive | tive | hood | hood |
| Model description | n (%) | n (%) | n (%) | n (%) | % | % | % | interval) | value, % | value, % | ratio | ratio |
| Model 1a: clinical | 48 | 1 | 68 | 16 | 98.0 | 81.0 | 89.5 | 0.95 | 75.0 | 98.6 | 5.1 | <0.1 |
| and proteomic | (98%) | (2%) | (81%) | (19%) | | | | (0.91-0.99) | | | | |
| Dataset: EU-GEI | | | | | | | | | | | | |
| initial experiment, | | | | | | | | | | | | |
| all sites | | | | | | | | | | | | |
| Features: 69 clinical | | | | | | | | | | | | |
| and 166 proteomic | | | | | | | | | | | | |
| Target: transition | | | | | | | | | | | | |
| status | | | | | | | | | | | | |
| N: 49 transition, 84 | | | | | | | | | | | | |
| non-transition | | | | | | | | | | | | |
| Model 1b: clinical | 23 | 26 | 45 | 39 | 46.9 | 53.6 | 50.3 | 0.48 | 37.1 | 63.4 | 1.0 | 1.0 |
| Dataset: EU-GEI | (47%) | (53%) | (54%) | (46%) | | | | (0.38-0.58) | | | | |
| initial experiment, | | | | | | | | | | | | |
| all sites | | | | | | | | | | | | |
| Features: 69 clinical | | | | | | | | | | | | |
| Target: transition | | | | | | | | | | | | |
| status | | | | | | | | | | | | |
| N: 49 transition, 84 | | | | | | | | | | | | |
| non-transition | | | | | | | | | | | | |
| Model 1c: proteomic | 49 | 0 | 71 | 13 | 100.0 | 84.5 | 92.3 | 0.96 | 79.0 | 100.0 | 6.5 | <0.1 |
| Dataset: EU-GEI | (100%) | (0%) | (85%) | (15%) | | | | (0.92-1.00) | | | | |
| initial experiment, | | | | | | | | | | | | |
| all sites | | | | | | | | | | | | |
| Features: 166 | | | | | | | | | | | | |
| proteomic | | | | | | | | | | | | |
| Target: transition | | | | | | | | | | | | |
| status | | | | | | | | | | | | |
| N: 49 transition, 84 | | | | | | | | | | | | |
| non-transition | | | | | | | | | | | | |
| Model 2a: proteomic | 28 | 2 | 40 | 10 | 93.3 | 80.0 | 86.7 | 0.94 | 73.7 | 95.2 | 4.7 | 0.1 |
| (non-London) | (93%) | (7%) | (80%) | (20%) | | | | (0.88-1.00) | | | | |
| Dataset: EU-GEI | | | | | | | | | | | | |
| initial experiment, | | | | | | | | | | | | |
| all sites except | | | | | | | | | | | | |
| London | | | | | | | | | | | | |
| Features: 166 | | | | | | | | | | | | |
| proteomic | | | | | | | | | | | | |
| Target: transition | | | | | | | | | | | | |
| status | | | | | | | | | | | | |
| N: 30 transition, 50 | | | | | | | | | | | | |
| non-transition | | | | | | | | | | | | |
| Model 2b: top 10, | 30 | 0 | 41 | 9 | 100.0 | 82.0 | 91.0 | 0.99 | 76.9 | 100.0 | 5.6 | <0.1 |
| training data | (100%) | (0%) | (82%) | (18%) | | | | (0.96-1.00) | | | | |
| Dataset: EU-GEI | | | | | | | | | | | | |
| initial experiment, | | | | | | | | | | | | |
| all sites except | | | | | | | | | | | | |
| London | | | | | | | | | | | | |
| Features: 10 | | | | | | | | | | | | |
| proteomic | | | | | | | | | | | | |
| Target: transition | | | | | | | | | | | | |
| status | | | | | | | | | | | | |
| N: 30 transition, 50 | | | | | | | | | | | | |
| non-transition | | | | | | | | | | | | |
| Model 2b: top 10, test | 18 | 1 | 30 | 4 | 94.7 | 88.2 | 91.5 | 0.92 | 81.8 | 96.8 | 8.1 | 0.1 |
| data | (95%) | (5%) | (88%) | (12%) | | | | (0.83-1.00) | | | | |
| Dataset: EU-GEI | | | | | | | | | | | | |
| initial experiment, | | | | | | | | | | | | |
| London site | | | | | | | | | | | | |
| Features: 10 | | | | | | | | | | | | |
| proteomic | | | | | | | | | | | | |
| Target: transition | | | | | | | | | | | | |
| status | | | | | | | | | | | | |
| N: 19 transition, 34 | | | | | | | | | | | | |
| non-transition | | | | | | | | | | | | |
| Model 3: replication | 48 | 1 | 77 | 9 | 98.0 | 89.5 | 93.7 | 0.98 | 84.2 | 98.7 | 9.4 | <0.1 |
| Dataset: EU-GEI | (98%) | (2%) | (90%) | (10%) | | | | (0.95-1.00) | | | | |
| replication | | | | | | | | | | | | |
| experiment, all sites | | | | | | | | | | | | |
| Features: 69 clinical | | | | | | | | | | | | |
| and 119 proteomic | | | | | | | | | | | | |
| Target: transition | | | | | | | | | | | | |
| status | | | | | | | | | | | | |
| N: 49 transition, 86 | | | | | | | | | | | | |
| non-transition | | | | | | | | | | | | |
| Model 4: ALSPAC | 40 | 15 | 47 | 19 | 72.7 | 71.2 | 72.0 | 0.74 | 67.8 | 75.8 | 2.5 | 0.4 |
| PEs | (73%) | (27%) | (71%) | (29%) | | | | (0.65-0.83) | | | | |
| Dataset: ALSPAC | | | | | | | | | | | | |
| Features: 265 | | | | | | | | | | | | |
| proteomic | | | | | | | | | | | | |
| Target: PEs age 18 | | | | | | | | | | | | |
| N: 55 PEs, 66 no PE | | | | | | | | | | | | |
| Model S1: ELISA | 33 | 11 | 51 | 31 | 75.0 | 62.2 | 68.6 | 0.76 | 51.6 | 82.3 | 2.0 | 0.4 |
| Dataset: EU-GEI | (75%) | (25%) | (62%) | (38%) | | | | (0.67-0.85) | | | | |
| initial experiment, | | | | | | | | | | | | |
| all sites | | | | | | | | | | | | |
| Features: 9 ELISA | | | | | | | | | | | | |
| Target: transition | | | | | | | | | | | | |
| status | | | | | | | | | | | | |
| N: 44 transition, 82 | | | | | | | | | | | | |
| non-transition | | | | | | | | | | | | |
| Model S2: functional | 27 | 20 | 22 | 10 | 57.4 | 68.8 | 63.1 | 0.74 | 73.0 | 52.4 | 1.8 | 0.6 |
| outcome | (57%) | (43%) | (69%) | (31%) | | | | (0.63-0.85) | | | | |
| Dataset: EU-GEI | | | | | | | | | | | | |
| initial experiment, | | | | | | | | | | | | |
| all sites | | | | | | | | | | | | |
| Features: 69 clinical | | | | | | | | | | | | |
| and 166 proteomic | | | | | | | | | | | | |
| Target: functional | | | | | | | | | | | | |
| outcome | | | | | | | | | | | | |
| N: 47 poor | | | | | | | | | | | | |
| functioning | | | | | | | | | | | | |
| (GAF ≤ 60); 32 good | | | | | | | | | | | | |
| functioning | | | | | | | | | | | | |
| (GAD > 60) | | | | | | | | | | | | |

AUC: area under the receiver-operating characteristic curve; EU-GEI: European Network of National Schizophrenia Networks Studying Gene-Environment Interactions; ALSPAC: Avon Longitudinal Study of Parents and Children; PEs: psychotic experiences. Models 1a-d, 2 and 3 are adjusted for age, sex, body mass index and years in education and Model 4 is additionally adjusted for ethnicity and tobacco use. Model 5 is adjusted for sex, maternal social class at birth and body mass index at age 12.

TABLE 18
Ten percent highest-weighted features for Model 1a, Model 3 and Model 4 (ranked according to mean feature weight
for models selected in cross-validation inner loop)

| Model 1a: EU-GEI clinical and | | | | | |
|---|---|---|---|---|---|
| proteomic data, initial experiment, | | Model 3: EU-GEI clinical and proteomic | | | |
| all sites | | data, replication experiment, all sites | | Model 4: ALSPAC proteomic data | |
| | Mean | | Mean | | Mean |
| Feature | weight | Feature | weight | Feature | weight |
| P01023 Alpha-2- | −0.330 | P01023 Alpha-2-macroglobulin | −0.286 | P04003 C4b-binding protein | −0.227 |
| macroglobulin | | | | alpha chain | |
| P01871 Immunoglobulin | −0.256 | P22792 Carboxypeptidase N | 0.210 | P27169 Serum | −0.180 |
| heavy constant mu | | subunit 2 | | paraoxonase/arylesterase 1 | |
| P04003 C4b-binding | −0.161 | P01871 Immunoglobulin heavy | −0.193 | Q03591 Complement factor H- | −0.152 |
| protein alpha chain | | constant mu | | related protein 1 | |
| P07357 Complement | 0.158 | P09871 Complement C1s | −0.181 | P07225 Vitamin K-dependent | −0.145 |
| component 8 alpha chain | | subcomponent | | protein S | |
| P55058 Phospholipid | −0.146 | P01011 Alpha-1-antichymotrypsin | 0.168 | P61626 Lysozyme C | −0.142 |
| transfer protein | | | | | |
| O75636 Ficolin-3 | −0.145 | P00747 Plasminogen | 0.163 | P55103 Inhibin beta C chain | 0.139 |
| P02774 Vitamin D | 0.135 | P08571 Monocyte differentiation | 0.161 | Q08380 Galectin-3-binding | 0.132 |
| binding protein | | antigen CD14 | | protein | |
| P07225 Vitamin K- | −0.132 | P10909 Clusterin | 0.158 | P24593 Insulin-like growth | 0.122 |
| dependent protein S | | | | factor-binding protein 5 | |
| P43320 Beta-crystallin | 0.132 | Q16610 Extracellular matrix | 0.157 | P00746 Complement factor D | 0.120 |
| B2 | | protein 1 | | | |
| P02766 Transthyretin | −0.130 | G3XAM2 Complement factor I | 0.140 | P01019 Angiotensinogen | −0.118 |
| P23142 Fibuln-1 | 0.125 | P04003 C4b binding protein alpha | −0.140 | P01871 Immunoglobulin heavy | −0.116 |
| | | chain | | constant mu | |
| P10909 Clusterin | 0.121 | P13671 Complement component 6 | 0.132 | O75636 Ficolin-3 | 0.115 |
| P05155 Plasma protease | −0.114 | P25311 Zinc alpha-2-glycoprotein | −0.131 | Q9H4A9 Dipeptidase 2 | −0.115 |
| C1 inhibitor | | | | | |
| Sex | −0.111 | P07359 Platelet glycoprotein Ib | 0.126 | P01023 Alpha-2-macroglobulin | −0.113 |
| | | alpha chain | | | |
| P00747 Plasminogen | 0.111 | P01031 Complement C5 | 0.125 | P04275 von Willebrand factor | −0.111 |
| P13671 Complement | 0.111 | O75882 Attractin | 0.123 | Q9NQ79 Cartilage acidic protein | 0.107 |
| component 6 | | | | 1 | |
| P02747 Complement C1q | 0.109 | P0DOY3 Immunoglobulin lambda | −0.120 | P24592 Insulin-like growth | 0.106 |
| subcomponent subunit C | | constant 3 | | factor-binding protein 6 | |
| P02753 Retinol-binding | 0.109 | P15169 Carboxypeptidase N | 0.115 | P09871 Complement C1s | −0.105 |
| protein 4 | | catalytic chain (CPN) | | subcomponent | |
| Q76LX8 A disintegrin | −0.108 | | | P10909 Clusterin | −0.105 |
| and metalloproteinase | | | | | |
| with thrombospondin | | | | | |
| motifs 13 | | | | | |
| P08697 Alpha-2- | −0.106 | | | O95497 Pantetheinase | 0.105 |
| antiplasmin | | | | | |
| P19827 Inter-alpha- | 0.105 | | | P02654 Apolipoprotein C-I | −0.099 |
| trypsin inhibitor heavy | | | | | |
| chain H1 | | | | | |
| MADRS: concentration | −0.104 | | | P02679 Fibrinogen gamma chain | −0.099 |
| difficulties | | | | | |
| P02489 Alpha-crystallin | 0.101 | | | P07358 Complement component | 0.097 |
| A chain | | | | C8 beta chain | |
| | | | | Q5T7F0 Neuropilin | −0.097 |
| | | | | P04040 Catalase | 0.094 |
| | | | | P43251 Biotinidase | 0.094 |

Proteins are presented with their Uniprot accession number and corresponding protein name.
EU-GEI: European Network of National Schizophrenia Networks Studying Gene-Environment Interactions; ALSPAC: Avon Longitudinal Study of Parents and Children; BPRS: Brief Psychiatric Rating Scale; MADRS: Montgomery-Asberg Depression Rating Scale; SANS: Scale for the Assessment of Negative Symptoms

TABLE 19
Ten percent highest-weighted features for Model S2
(support vector machine model predicting functional
outcome at 24 months in EU-GEI)

| | Mean |
|---|---|
| Feature | weight |
| BPRS: suspiciousness | 0.197 |
| P01023 Alpha-2-macroglobulin | −0.191 |
| P55058 Phospholipid transfer protein | −0.186 |
| P01871 Immunoglobulin heavy constant | −0.182 |
| mu | |
| Q9UGM5 Fetuin-B | 0.148 |
| O43866 CD5 antigen-like | −0.145 |
| P14618 Pyruvate kinase | −0.133 |
| P19827 Inter-alpha-trypsin inhibitor heavy | 0.129 |
| chain H1 | |
| SANS: blocking | 0.118 |
| SANS: increased latency of response | 0.111 |
| P10909 Clusterin | 0.107 |
| P08603 Complement factor H | 0.104 |
| P36955 Pigment epithelium-derived factor | 0.103 |
| MADRS: suicidal thoughts | −0.103 |
| Impersistence at work or school | 0.102 |
| P17936 Insulin-like growth factor-binding | 0.102 |
| protein 3 | |
| Age | −0.101 |
| P04196 Histidine-rich glycoprotein | 0.099 |
| Q08380 Galectin-3-binding protein | 0.097 |
| SANS: grooming and hygiene | 0.097 |
| SANS: ability to feel intimacy and | 0.095 |
| closeness | |
| SANS: sexual activity | −0.093 |
| P11226 Mannose-binding protein C | 0.090 |

Features are ranked according to mean feature weight for models selected in the cross-validation inner loop.
EU-GEI: European Network of National Schizophrenia Networks Studying Gene-Environment Interactions; BPRS: Brief Psychiatric Rating Scale; SANS: Scale for Assessment of Negative Symptoms; MADRS: Montgomery-Asberg Depression Rating Scale.

1. A method of determining the likelihood of an individual transitioning to a first episode of psychosis (FEP), the method comprising:
determining the level of markers in a bodily fluid sample from the individual, wherein the markers are selected from one or more proteins of Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin Antithrombin III, and N-acetylmuramoyl-L-alanine amidase,
wherein an increase in the level of one or more markers selected from Complement component 8 alpha chain, Complement component 6, Retinol-binding protein 4, Beta-crystallin B2, Vitamin D binding protein, Inter-alpha-trypsin inhibitor heavy chain H1, Fibulin-1, Clusterin, L-lactate dehydrogenase B chain, Complement C1q subcomponent subunit C, and Alpha-crystallin A chain, coagulation factor XII, Carboxypeptidase N subunit 2, Alpha 1 anti-chymotrypsin, Plasminogen, Monocyte differentiation antigen CD14, Attractin, Zinc alpha-2-glycoprotein, Extracellular matrix protein 1, Complement C1s subcomponent, Ceruloplasmin, Antithrombin III and Complement Factor I; and/or a decrease in the level of one or more markers selected from Alpha-2-macroglobulin, Immunoglobulin heavy constant mu, Phospholipid transfer protein, C4b-binding protein alpha chain, Vitamin K-dependent protein S, Ficolin-3, Transthyretin, Plasma protease C1 inhibitor, Alpha-2-antiplasmin, A disintegrin and metalloproteinase with thrombospondin motifs 13, Immunoglobulin lambda constant 3, and N-acetylmuramoyl-L-alanine amidase; is predictive of the individual transitioning to a first episode of psychosis (FEP).

2. The method according to claim 1, wherein the individual is an ultra-high risk (UHR) individual for psychosis.

3. The method according to claim 1 or claim 2, further comprising the assessment of clinical features.

4. The method according to any preceding claim, further comprising selecting the individual for therapeutic intervention and/or a follow-up check, if the individual is predicted to transition to a first episode of psychosis (FEP) and/or develop a functional disability.

5. The method according to any preceding claim, further comprising administering a therapeutic or preventative medication to the individual, if the individual is predicted to transition to a first episode of psychosis (FEP).

6. A method of predicting the functional outcome for an individual following a first episode of psychosis (FEP), the method comprising:
determining the level of markers in a bodily fluid sample from the individual, wherein the markers are selected from one or more proteins of
Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C, wherein an increase in the level of one or more markers selected from Fetuin-B, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C; and/or a decrease in the level of one or more markers selected from Alpha-2-macroglobulin, Phospholipid transfer protein, Immunoglobulin heavy constant mu, CD5 antigen-like, and Pyruvate kinase, is predictive of an increased risk of functional disability outcome for the individual.

7. The method according to claim 6, further comprising the assessment of clinical features.

8. The method according to claim 7, wherein the method comprises the further assessment of one or more, or all, of the clinical features selected from BPRS: suspiciousness, SANS: impersistence at work or school, SANS: increased latency of response, SANS: blocking, SANS: grooming and hygiene, BPRS: excitement, SANS: sexual activity, and MADRS: suicidal thoughts.

9. The method according to any one of claims 6-8, further comprising selecting the individual for therapeutic intervention and/or a follow-up check, if the individual is predicted to develop a functional disability.

10. The method according to any one of claims 6-9, further comprising administering a therapeutic or preventative medication to the individual, if the individual is predicted to develop a functional disability.

11. The method according to any preceding claim, wherein determining the level of a marker comprises conducting an enzyme-linked immunosorbent assay (ELISA) to determine the level of one or more markers in the sample or by a Proximity Extension Assay (PEA).

12. The method according to any preceding claim, wherein the markers are detected by probes that are immobilised on a substrate.

13. A composition comprising a plurality (e.g. two or more) of probes capable of binding to protein markers in a bodily fluid sample, wherein the protein markers comprise two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase, Immunoglobulin heavy constant mu, Fetuin-B, CD5 antigen-like, Pyruvate kinase, Complement factor H, Pigment epithelium-derived factor, Insulin-like growth factor-binding protein 3, Histidine-rich glycoprotein, Galectin-3-binding protein, and Mannose-binding protein C.

14. The composition according to claim 13, wherein the composition comprises a plurality of probes capable of binding to protein markers in a bodily fluid sample, wherein the protein markers comprise two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

15. The composition according to claim 13 or 14, wherein a plurality of probes are provided for binding to one or more, or all, of the protein markers selected from Alpha-2-macroglobulin, Immunoglobulin heavy constant mu, C4b-binding protein alpha chain, Phospholipid transfer protein, Transthyretin, Vitamin D binding protein, Beta-crystallin B2, Vitamin K-dependent protein S, Coagulation factor XII and clusterin; or
wherein a plurality of probes are provided for binding to one or more, or all, of the protein markers selected from alpha-2-macroglobulin (A2M), immunoglobulin heavy constant mu (IGHM), C4b-binding protein alpha chain (C4BPA), vitamin K-dependent protein S, fibulin-1, transthyretin, N-acetylmuramoyl-L-alanine amidase, vitamin D-binding protein, clusterin and complement component 6 (C6); or
wherein a plurality of probes are provided for binding to one or more, or all, of the protein markers selected from alpha-2-macroglobulin, Immunoglobulin heavy constant mu, C4b-binding protein alpha chain, complement component 8 alpha chain, Phospholipid transfer protein, ficolin-3, vitamin D binding protein, vitamin K-dependent protein S, beta-crystallin B2, and transthyretin.

16. The composition according to claim 13, wherein a plurality of probes are provided for binding to protein markers in a bodily fluid sample, wherein the protein markers comprise two or more proteins selected from the group comprising
Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C.

17. The composition according to claim 16, wherein a plurality of probes are provided for binding to protein markers in a bodily fluid sample, wherein the protein markers comprise two or more proteins selected from the group comprising
Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, and Pigment epithelium-derived factor.

18. The composition according to any of claims 13-17, wherein the probes are provided as a panel of probes anchored to a surface.

19. A method of detecting the level of two or more proteins selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase, in a bodily fluid sample; and optionally the bodily fluid sample may be of an ultra-high risk (UHR) individual for psychosis.

20. A method of detecting the level of two or more proteins selected from the group comprising
Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C,
in a bodily fluid sample from an individual; and
optionally the bodily fluid sample may be of a ultra-high risk (UHR) individual for psychosis.

21. A method for treating an individual to prevent a transition to a FEP, the method comprising the steps of:
determining whether the individual is predicted to transition to a FEP by:
obtaining or having obtained a sample from the individual; and
performing or having performed the method according to any one of claims 1-12 to determine if the individual is predicted to transition to a FEP; and
if the individual is predicted to transition to a FEP, then administering medication as described herein to the individual.

22. A method for treating an individual to prevent a transition to a FEP, the method comprising the steps of:
receiving results of a test performed according to the method of any one of claims 1-12 to determine if the individual is predicted to transition to a FEP; and
if the individual is predicted to transition to a FEP, then administering medication as described herein to the individual.

23. A method for treating an individual to prevent a functional disability following a FEP, the method comprising the steps of:
determining whether the individual is predicted to develop a functional disability by:
obtaining or having obtained a sample from the individual; and
performing or having performed the method according to any one of claims 1-12 to determine if the individual is predicted to develop a functional disability; and
if the individual is predicted to develop a functional disability, then administering medication as described herein to the individual.

24. A method for treating an individual to prevent the development of a functional disability following a FEP, the method comprising the steps of:
receiving results of a test performed according to the method according to any one of claims 1-12 to determine if the individual is predicted to develop a functional disability; and
if the individual is predicted to develop a functional disability, then administering medication as described herein to the individual.

25. Use of one or more proteins as a predictive biomarker for an individual to transition to a FEP, wherein the protein is selected from the group comprising Alpha-2-macroglobulin, Immunoglobulin heavy constant mu, Phospholipid transfer protein, C4b-binding protein alpha chain, Complement component 8 alpha chain, Vitamin K-dependent protein S, Ficolin-3, Transthyretin, Complement component 6, Retinol-binding protein 4, Beta-crystallin B2, Vitamin D binding protein, Inter-alpha-trypsin inhibitor heavy chain H1, Plasma protease C1 inhibitor, Alpha-2-antiplasmin, Fibulin-1, Clusterin, L-lactate dehydrogenase B chain, Extracellular matrix protein 1, disintegrin and metalloproteinase with thrombospondin motifs 13, Complement C1q subcomponent subunit C, and Alpha-crystallin A chain, coagulation factor XII, Carboxypeptidase N subunit 2, Complement C1s subcomponent, Alpha 1 anti-chymotrypsin, Plasminogen, Monocyte differentiation antigen CD14, Zinc alpha-2-glycoprotein, Attractin, Complement Factor I, Immunoglobulin lambda constant 3, Ceruloplasmin, Antithrombin III and N-acetylmuramoyl-L-alanine amidase.

26. Use of one or more proteins as a predictive biomarker for predicting the likelihood of a functional disability outcome for an individual following a FEP, wherein the protein is selected from the group comprising:
Alpha-2-macroglobulin, Phospholipid transfer protein, Immunoglobulin heavy constant mu, Fetuin-B, CD5 antigen-like, Pyruvate kinase, Inter-alpha-trypsin inhibitor heavy chain H1, Clusterin, Complement factor H, Pigment epithelium-derived factor, Insulin-like growth factor-binding protein 3, Histidine-rich glycoprotein, Galectin-3-binding protein, and Mannose-binding protein C.

27. The use according to any of claim 25 or 26, wherein the use is determining the levels of the protein(s) in a bodily fluid sample from the individual, optionally wherein the individual is an UHR individual for psychosis.
